(19) United States
(12) Patent Application Publication
(10) Pub. No.: US 2017/0360875 A1
(43) Pub. Date: Dec. 21, 2017

(54) METHODS FOR TREATING IMMUNE-MEDIATED VIRAL INFECTIONS

(71) Applicants: Neil M. BODIE, Temple City, CA (US); Hyo PARK, Shelbyville, TN (US); Elliot ALTMAN, Rockvale, TN (US); MIDDLE TENNESSEE STATE UNIVERSITY, Murfreesboro, TN (US)

(72) Inventors: Neil M. Bodie, San Gabriel, CA (US); Hyo Park, Shelbyville, TN (US); Elliot Altman, Rockvale, TN (US)

(73) Assignee: MIDDLE TENNESSEE STATE UNIVERSITY, Murfreesboro, TN (US)

(21) Appl. No.: 15/538,682

(22) PCT Filed: Dec. 22, 2015
(86) PCT No.: PCT/US2015/067433

§ 371 Date: Jun. 22, 2017

Related U.S. Application Data
(60) Provisional application No. 62/208,301, filed on Aug. 21, 2015.
(60) Provisional application No. 62/124,485, filed on Dec. 22, 2014.

(51) Int. Cl. A61K 38/10; A61K 38/08; A61K 31/00

(52) U.S. Cl. CPC A61K 38/10; A61K 38/08; A61K 31/00

(57) ABSTRACT

Polypeptides and other compounds that can bind specifically to the C_{H}2-C_{H}3 cleft of an immunoglobulin molecule, as well as methods for using such polypeptides and compounds to inhibit Fc-mediated immune complex formation in viral infection, I are described. For example, polypeptides and other compounds can be used to inhibit immune complex formation in a subject who has been diagnosed as having or is suspected of having an Ebola virus infection, a subject who has been diagnosed as having or is suspected of having an influenza virus infection, or a subject who has been diagnosed as having or is suspected of having a hepatitis virus infection.

### CONTINUING APPLICATION DATA

This application claims the benefit of U.S. Provisional Application Ser. No. 62/124,485, filed Dec. 22, 2014, and U.S. Provisional Application Ser. No. 62/208,301, filed Aug. 21, 2015, each of which is incorporated by reference herein.

### SEQUENCE LISTING

The instant application contains a Sequence Listing which has been submitted electronically in ASCII format and is hereby incorporated by reference in its entirety. Said ASCII copy, created on Dec. 17, 2015, is named 504.00030201_SL.txt and is 22,591 bytes in size.

### BACKGROUND

Humoral immune responses are triggered when an antigen binds specifically to an antibody. The combination of an antibody molecule and an antigen forms a small, relatively soluble immune complex. Antigens either can be foreign substances, such as viral or bacterial polypeptides, or can be “self-antigens” such as polypeptides normally found in the human body. The immune system normally distinguishes foreign antigens from self-antigens. “Autoimmune” disease can occur, however, when this system breaks down, such that the immune system turns upon the body and destroys tissues or organ systems as if they were foreign substances. Larger immune complexes are more pathogenic than small, more soluble immune complexes. The formation of large, relatively insoluble immune complexes can result from both the interaction of antibody molecules with antigen and the interaction of antibody molecules with each other. Such immune complexes also can result from interactions between antibodies in the absence of antigen.

Antibodies can prevent infections by coating viruses or bacteria, but otherwise are relatively harmless by themselves. In contrast, organ specific tissue damage can occur when antibodies combine with antigens and the resulting immune complexes bind to certain effector molecules in the body. Effector molecules are so named because they carry out the pathogenic effects of immune complexes. By inhibiting the formation of large, insoluble immune complexes, or by inhibiting the binding of immune complexes to effector molecules, the tissue damaging effects of immune complexes may be prevented.

### SUMMARY OF THE INVENTION

This document is based in part on the discovery that polypeptides having amino acid sequences based on those set forth in SEQ ID NO:2 and SEQ ID NO:20 (also referred to herein as NB406) can bind specifically and with high affinity to the C_{H}2-C_{H}3 domain of immunoglobulin molecules, thus inhibiting the formation of insoluble immune complexes containing antibodies and antigens, and preventing the binding of such complexes to effector molecules. This document provides such polypeptides, other C_{H}2-C_{H}3 binding compounds, compositions containing the polypeptides and/or compounds, and methods for using the polypeptides and compositions to inhibit immune complex formation and therapeutic use in treating viral infections.

In some embodiments, the polypeptides may inhibit or prevent the formation of an Fc receptor to an immune complex that includes an antibody and an antigen. That is, polypeptides may inhibit or prevent the binding of a protein that could otherwise bind to the Fc-portion of an antibody, including an antibody bound to an antigen, to the Fc-portion of the antibody.

In one aspect, this document features a method for inhibiting immune complex formation in a subject, the method comprising administering to the subject a composition comprising a purified polypeptide, the polypeptide comprising the amino acid sequence (Xaa₁)ₘ-Cys-Ala-Xaa₂-His-Leu-Gly-Glu-Leu-Val-Trp-Cys-Thr-(Xaa₃)ₙ (SEQ ID NO:60), wherein Xaa₁ is any amino acid, Xaa₂ is Trp, Tyr or Phe, 5-Hydroxytrphophan (5-HTP), 5-hydroxytryptamine (5-HT), or another amino acid derivative, Xaa₃ is any amino acid, and m and n independently are 0, 1, 2, 3, 4, or 5, and wherein the immune complex formation is associated with a viral infection. The immune complex formation can be associated with Antibody Dependent Enhancement (ADE) of Ebola Fever or infection by Ebola Virus (EBOV), or contributes to the enhancement of an EBOV infection. The peptide can cause clinical or histiological improvement of an EBOV infection. The peptide can cause an improvement in or delay the onset of one or more of the histiological characteristics of an EBOV infection. The peptide can decrease the ADE of an EBOV infection. In some embodiments, the subject may have been diagnosed as having or may be suspected of having an EBOV infection. As used herein, the terms “EBOV infection” and “Ebola virus infection” are inclusive of Ebola Virus Disease (EVD) and the more severe manifestation of EVD, Ebola Hemorrhagic Fever (EHF). In one or more embodiments, the subject may be exhibiting symptoms of an EBOV infection, the subject may be at risk of contracting an EBOV infection, and/or the subject may have been exposed to EBOV infection. The EBOV may include, for example, Ebola virus Zaire (Zaire ebolavirus; ZEBOV); Sudan virus (Sudan ebolavirus; SUDV or SEBOV); Tai Forest virus (Taï Forest ebolavirus; TAFV); Bundibugyo virus (Bundibugyo ebolavirus; BDBV); or Reston virus (Reston ebolavirus; RESTV or REBOV). In one or more embodiments, the subject may be a human. Additionally, the subject may have or be suspected of having a coinfection with a pathogen other than EBOV including, for example, human immunodeficiency virus (HIV), human cytomegalovirus (HCMV), herpes simplex virus (HSV), hepatitis C virus, human papilloma virus (HPV), Mycobacterium tuberculosis, malaria/Plasmodium falciparum, and/or Schistosoma haematobium. In one or more embodiments, the subject may be a pig or non-human primate. Additionally, the subject may have or be suspected of having a coinfection with a pathogen other than EBOV including, for example porcine reproductive and respiratory syndrome virus (PRRSV).

In some embodiments, the subject may have been diagnosed as having or may be suspected of having an influenza virus infection. In one or more embodiments, the subject may be exhibiting symptoms of an influenza infection, the subject may be at risk of contracting an influenza infection, and/or the subject may have been exposed to influenza. Additionally, the subject may have or be suspected of having an autoimmune disease. In one or more embodiments, the subject may be a human.

In some embodiments where the subject has been diagnosed as having or is suspected of having an influenza virus infection and where immune complex formation contributes to the enhancement of the influenza virus infection, the method includes administering to the subject a composition including a purified polypeptide. The polypeptide can include the amino acid sequence Xaa-Pro-Pro-Asp-Cys-Ala-Trp-His-Leu-Gly-Glu-Leu-Val-Trp-Cys-Thr (SEQ ID NO: 19), wherein Xaa is any amino acid.

In some embodiments where the subject who has been diagnosed as having or is suspected of having a hepatitis virus infection and where immune complex formation contributes to the enhancement of the hepatitis virus infection, the method includes administering to the subject a composition including a purified polypeptide. The polypeptide can include the amino acid sequence Xaa-Pro-Pro-Asp-Cys-Ala-Trp-His-Leu-Gly-Glu-Leu-Val-Trp-Cys-Thr (SEQ ID NO: 19), wherein Xaa is any amino acid.

In some embodiments, the subject may have been diagnosed as having or may be suspected of having a hepatitis virus infection. In one or more embodiments, the subject may be exhibiting symptoms of a hepatitis infection, the subject may be at risk of contracting a hepatitis infection, and/or the subject may have been exposed to a hepatitis virus. Additionally, the subject may have or be suspected of having an autoimmune disease. In one or more embodiments, the subject may be a human.

The peptide can inhibit binding of a heterologous immune complex (IC), that is, a complex formed between a non-Ebola antibody and a non-Ebola antigen, to an FcγR. The peptide can inhibit binding of an EBOV-IgG IC, that is, an IC formed between an anti-Ebola antibody and an Ebola antigen, to an FcγR. The peptide can inhibit formation of ICs that contribute to immunopathogenesis of the ADE of EBOV infections. The peptide can inhibit binding of EBOV virions to IgG IC. The peptide can inhibit binding of an EBOV protein (e.g., glycoprotein (GP)) to an IgG immune complex. The peptide can inhibit binding of a heterologous IC and/or an EBOV-IgG IC to FcγI, FcγIIa H131 allele, FcγIIa R131 allele, FcγRIIb, Fc.γRIIc, FcγRIIIa, or FcγIIIb. The peptide can inhibit binding of a heterologous IC and/or EBOV-IgG IC to mC1q, sC1q, or FcRn.

The polypeptide can comprise a terminal stabilizing group. The terminal stabilizing group can be at the amino terminus of the polypeptide and can be a tripeptide having the amino acid sequence Xaa-Pro-Pro, wherein Xaa is any amino acid (e.g., Ala). The terminal stabilizing group can be at the carboxy terminus of the polypeptide and can be a tripeptide having the amino acid sequence Pro-Pro-Xaa, wherein Xaa is any amino acid (e.g., Ala).

The method can further comprise the step of monitoring the subject for one or more clinical, histiopathological or molecular characteristics of hemorrhagic fever, influenza virus infection, or hepatitis virus infection. The one or more clinical, histiopathological, or molecular characteristics of hemorrhagic fever can be selected from the group consisting of a decrease in platelets, hemoconcentration, or an increase in FcγR+ effector cells.

The polypeptide can comprise the amino acid sequence Xaa-Pro-Pro-Asp-Cys-Ala-Trp-His-Leu-Gly-Glu-Leu-Val-Trp-Cys-Thr (SEQ ID NO: 19), wherein Xaa is any amino acid. The polypeptide can comprise the amino acid sequence Ala-Pro-Pro-Asp-Cys-Ala-Trp-His-Leu-Gly-Glu-Leu-Val-Trp-Cys-Thr (SEQ ID NO:20). The polypeptide can comprise the amino acid sequence Asp-Cys-Ala-Trp-His-Leu-Gly-Glu-Leu-Val-Trp-Cys-Thr (SEQ ID NO:2).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below.

The details of one or more embodiments of the invention are set forth in the accompanying drawings and the description below. Other features, objects, and advantages of the invention will be apparent from the description and from the claims.

### BRIEF DESCRIPTION OF THE FIGURES

FIG. 1A shows the clinical burden for serious Malaria in the countries of Liberia, Sierra Leone, and Guinea. (Malaria Atlas Project, available on the world wide web at map.ox.ac.uk). FIG. 1B shows a map of the reported Ebola outbreaks from the CDC, as of the end of October 2014 (MMWR CDC Nov. 18 2014 Ebola Virus Disease Epidemic—West Africa November 2014, available on the world wide web at cdc.gov/mmwr/pdf/wk/mm63e1118.pdf). FIG. 1C shows a composite map of Ebola cases and serious Malaria cases. FIG. 1D shows a graphical representation of the incidence of malarial deaths and Ebola cases (Walker et al., Lancet Infectious Dis 2015 Epub Apr. 24, 2015, p. 4.).

FIG. 2 shows Ebola virus's viral FcR binding to the Fc portion of an antibody. The Fc portion of the antibody is also bound to cellular FcγR. FcRIIa/CD32a (GOLD); IgG Fc (RED & GREEN); EBOV viral FcR (BLUE).

FIG. 3 shows an anti-malaria IgG immune complex where the IgG is also bound to human FcR and ZEBOV GP. The IgG is shown in green, light and dark blue; malaria PfEMP1 is shown in gold; hFcR is shown in red; and ZEBOV GP is shown in purple.

FIG. 4A shows a “chalice”-like structure formed by three GP1/GP2 molecules forming a trimer. FIG. 4B shows a ring structure formed by IgG hexamers that allows high affinity binding by C1q. FIG. 4C shows a model of Ebola GP binding to two or three IgGs present in an IgG hexamer.

### DETAILED DESCRIPTION OF ILLUSTRATIVE EMBODIMENTS

This document provides polypeptides and other compounds capable of interacting with the C_{H}2-C_{H}3 cleft of an immunoglobulin molecule, such that interaction of the immunoglobulin with other molecules (e.g., effectors or other immunoglobulins) is blocked. Methods for identifying such polypeptides and other compounds also are described, along with compositions and articles of manufacture containing the polypeptides and compounds. In addition, this document provides methods for using the polypeptides and compounds to inhibit immune complex formation and to treat diseases (e.g., viral diseases such as Ebola Virus Disease) in which IgG immune complexes bind to effector molecules, such as membrane bound C1q (mC1q), soluble C1q (sC1q), and FcγR5 (including, but not limited to FcγRI (and isoforms of FcγRs), FcγRIIa, FcγRIIb/c, FcγRIIIa, FcγRIIIb, and FcRn).

This document further provides methods of treating a subject diagnosed as having or suspected of having a hepatitis virus infection, an influenza virus infection, and/or an Ebola virus infection. Unexpectedly, and as further described in the Examples, Ebola virus, hepatitis virus, influenza virus and/or a surface protein of Ebola virus, hepatitis virus, and influenza virus can act as an Fc Receptor. The Fc receptor (FcR) activity of these viruses can facilitate interactions between the virus and a host cell receptor.

As used herein, an “Fc receptor” refers to a protein that can bind to the Fc-portion of an antibody, including an antibody bound to an antigen and/or an antibody that is not bound to an antigen. The Fc receptor (FcR) activity of these viruses can facilitate interactions between the virus and a host cell receptor. As used herein, an “immune complex” or “IC” refers both a complex between an immunoglobulin and an antigen and a complex between an immunoglobulin Fc region and other antibodies or factors. Specifically, an “Fc-mediated immune complex” refers to a complex between an immunoglobulin Fc region and other antibodies or factors including, for example, an Fc receptor. The Fc-mediated immune complex may, optionally, include bound antigen. As used herein, the terms “heterologous immune complex” and “heterologous IC” mean a complex formed between a non-Ebola antibody and a non-Ebola antigen.

An immunoglobulin that is bound to both a host cell Fc receptor and a viral Fc receptor can enhance binding and/or entry of a virus into to a host cells. Because the interaction between the host cell and the virus is facilitated by interactions with the Fc-portion of the antibody in an immunoglobulin complex, a heterologous immune complex, not just an anti-viral immune complex, can bridge a host cell-virus interaction. The previously unreported and surprising ability of hepatitis virus, influenza virus, and Ebola virus to interact with heterologous immune complexes may contribute to accelerated disease progression and increased infectivity, particularly in subjects having an increased number of circulating immune complexes.

By administering polypeptides and other compounds capable of interacting with the C_{H}2-C_{H}3 cleft of an immunoglobulin molecule, including, for example, SEQ ID NO: 19, SEQ ID NO:20, or SEQ ID NO:2, the interaction of the immunoglobulin with other molecules, including a host cell Fc receptor and/or a viral Fc receptor can be blocked or abrogated. When an immunoglobulin is part of an antibody-antigen immune complex, such abrogation can interfere with the immune complex's ability to bridge virus-host cell interactions.

### Immunoglobulins

The immunoglobulins make up a class of proteins found in plasma and other bodily fluids that exhibit antibody activity and bind to other molecules (e.g., antigens and certain cell surface receptors) with a high degree of specificity. Based on their structure and biological activity, immunoglobulins can be divided into five classes: IgM, IgG, IgA, IgD, and IgE. IgG is the most abundant antibody class in the body; this molecule assumes a twisted “Y” shape configuration. With the exception of the IgMs, immunoglobulins are composed mainly of four peptide chains that are linked by several intrachain and interchain disulfide bonds. For example, the IgGs are composed of two polypeptide heavy chains (H chains) and two polypeptide light chains (L chains), which are coupled by disulfide bonds and non-covalent bonds to form a protein molecule with a molecular weight of approximately 150,000 daltons (Saphire et al., “Crystal Structure of a Neutralizing Human IgG Against HIV-1: A Template for Vaccine Design,” Science, 2001, 293:1155-1159). The average IgG molecule contains approximately 4.5 interchain disulfide bonds and approximately 12 intrachain disulfide bonds (Frangione and Milstein (1968) J. Mol. Biol. 33:893-906).

The light and heavy chains of immunoglobulin molecules are composed of constant regions and variable regions (see, e.g., Padlan (1994) Mol. Immunol. 31:169-217). For example, the light chains of an IgG1 molecule each contain a variable domain (V_{L}) and a constant domain (C_{L}). The heavy chains each have four domains: an amino terminal variable domain (V_{H}), followed by three constant domains (C_{H}1, C_{H}2, and the carboxy terminal C_{H}3). A hinge region corresponds to a flexible junction between the C_{H}1 and C_{H}2 domains. Papain digestion of an intact IgG molecule results in proteolytic cleavage at the hinge and produces an Fc fragment that contains the C_{H}2 and C_{H}3 domains, and two identical Fab fragments that each contain a C_{H}1, C_{L}, V_{H}, and V_{L} domain. The Fc fragment has complement- and tissue-binding activity, while the Fab fragments have antigen-binding activity.

Immunoglobulin molecules can interact with other polypeptides through various regions. The majority of antigen binding, for example, occurs through the V_{L}/V_{H} region of the Fab fragment. The hinge region also is thought to be important, as immunological dogma states that the binding sites for Fc receptors (FcR) are found in the hinge region of IgG molecules (see, e.g., Raghavan and Bjorkman (1996) Annu. Rev. Dev. Biol. 12:181-200). More recent evidence, however, suggests that FcR interacts with the hinge region primarily when the immunoglobulin is monomeric (i.e., not immune-complexed). Such interactions typically involve the amino acids at positions 234-237 of the Ig molecule (Wiens et al. (2000) J. Immunol. 164:5313-5318).

Immunoglobulin molecules also can interact with other polypeptides through a cleft within the C_{H}2-C_{H}3 domain. The “C_{H}2-C_{H}3 cleft” typically includes the amino acids at positions 251-255 within the C_{H2} domain and the amino acids at positions 424-436 within the C_{H}3domain. As used herein, numbering is with respect to an intact IgG molecule as in Kabat et al. (Sequences of Proteins of Immunological Interest, 5^{th} ed., Public Health Service, U.S. Department of Health and Human Services, Bethesda, Md.). The corresponding amino acids in other immunoglobulin classes can be readily determined by those of ordinary skill in the art.

The C_{H}2-C_{H}3 cleft is unusual in that it is characterized by both a high degree of solvent accessibility and a predominantly hydrophobic character, suggesting that burial of an exposed hydrophobic surface is an important driving force behind binding at this site. A three-dimensional change occurs at the IgG C_{H}2-C_{H}3 cleft upon antigen binding, allowing certain residues (e.g., a histidine at position 435) to become exposed and available for binding. Direct evidence of three-dimensional structural changes that occur upon antigen binding was found in a study using monoclonal antibodies sensitive to conformational changes in the Fc region of human IgG. Five IgG epitopes were altered by antigen binding: two within the hinge region and three within the C_{H}2-C_{H}3 cleft (Girkontraite et al. (1996) Cancer Biother. Radiopharm. 11:87-96). Antigen binding therefore can be important for determining whether an immunoglobulin binds to other molecules through the hinge or the Fc C_{H}2-C_{H}3 region.

The Fc region can bind to a number of effector molecules and other proteins, including the following:
(1) FcRn—The neonatal Fc receptor determines the half-life of the antibody molecule in the general circulation (Leach et al., J. Immunol., 1996, 157:3317-3322; Gheti and Ward Ann. Rev. Immunol., 2000, 18:739-766). Mice genetically lacking FcRn are protected from the deleterious effects of pathogenic autoantibodies due to the shortened half-life of the autoantibodies (Liu et al., J Exp. Med., 1997, 186:777-783). The only binding site of FcRn to the IgG Fc is the IgG Fc C_{H}2-C_{H}3 cleft and HIS 435 has been shown by 3D structure and alanine scan to be essential to FcRn to IgG Fc binding (Shields et al. J Biol. Chem., 2001 276:6591-6604 and Martin et al., Mol. Cell, 2001, 7:867-877). Since the peptides described herein bind with high affinity to the C_{H}2-C_{H}3 cleft and HIS 435, the peptides are direct inhibitors of (immune complexed) IgG Fc to FcRn binding. An inhibitor of FcRn binding to immune complexes or to pathogenic autoantibodies would be useful in treating diseases involving pathogenic autoantibodies and/or immune complexes.
(2) FcR—The cellular Fc Receptor provides a link between the humoral immune response and cell-mediated effector systems (Hamano et al., J. Immunol., 2000 164:6113-6119; Coxon et al., Immunity, 2001, 14:693-704; Fossati et al., Eur. J. Clin. Invest., 2001, 31:821-831). The Fcγ. Receptors are specific for IgG molecules, and include FcγRI, FcγRIIa, FcγRIIb/c, and FcγRIIIa/b (and alleles, phenotypes and genotypes thereof). These isotypes bind with differing affinities to monomeric and immune-complexed IgG.
(3) C1q—The first component of the classical complement pathway is C1, which exists in blood serum as a complex of three proteins, C1q, C1r, and C1s. The classical complement pathway is activated when C1q binds to the Fc regions of antigen-bound IgG or IgM. Although the binding of C1q to a single Fc region is weak, C1q can form tight bonds to a cluster of Fc regions (Diebolder et al., Science, 2014, 343(6176):1260-1263). At this point C1 becomes proteolytically active.

The formation of immune complexes via interactions between immunoglobulin Fc regions and other antibodies or other factors (e.g., those described above) is referred to herein as “Fc-mediated immune complex formation” or “the Fc-mediated formation of an immune complex.” Immune complexes containing such interactions are termed “Fc-mediated immune complexes.” Fc-mediated immune complexes can include immunoglobulin molecules with or without bound antigen, and typically include C_{H}2-C_{H}3 cleft-specific ligands that have higher binding affinity for immune complexed antibodies than for monomeric antibodies.

### Purified Polypeptides

As used herein, a “polypeptide” is any chain of amino acid residues, regardless of post-translational modification (e.g., phosphorylation or glycosylation). The polypeptides provided herein typically are between 10 and 50 amino acids in length (e.g., 10, 11, 12, 13, 14, 15, 20, 25, 30, 35, 40, 45, or 50 amino acids in length). Polypeptides that are between 10 and 20 amino acids in length (e.g., 10, 11, 12, 13, 14, 15, 16, 17, 18, 19, or 20 amino acids in length) can be particularly useful.

The amino acid sequences of the polypeptides provided herein are somewhat constrained, but can have some variability. For example, the polypeptides provided herein typically include the amino acid sequence Xaa₁-Cys-Ala-Xaa₂-His-Xaa₃-Xaa₄-Xaa₅-Leu-Val-Trp-Cys-Xaa₆ (SEQ ID NO: 1), wherein the residues denoted by Xaaₙ can display variability. For example, Xaa₁ can be absent or can be any amino acid (e.g., Arg or Asp). Xaa₂ can be Phe, Tyr, Trp, 5-Hydroxytryptophan (5-HTP), or Arg. Xaa₃ can be any amino acid. Xaa₄ can be Gly or Ala, while Xaa₅ can be Glu or Ala. Like Xaa₁, Xaa₆ also can be absent or can be any amino acid (SEQ ID NO:57).

In one embodiment, a polypeptide can include the amino acid sequence Asp-Cys-Ala-Trp-His-Leu-Gly-Glu-Leu-Val-Trp-Cys-Thr (SEQ ID NO:2). Alternatively, a polypeptide can include the amino acid sequence Asp-Cys-Ala-Phe-His-Leu-Gly-Glu-Leu-Val-Trp-Cys-Thr (SEQ ID NO:3) or Asp-Cys-Ala-Arg-His-Leu-Gly-Glu-Leu-Val-Trp-Cys-Thr (SEQ ID NO:4). In another embodiment, a polypeptide can include the amino acid sequence Arg-Cys-Ala-Arg-His-Leu-Gly-Glu-Leu-Val-Trp-Cys-Thr (SEQ ID NO:5), Arg-Cys-Ala-Trp-His-Leu-Gly-Glu-Leu-Val-Trp-Cys-Thr (SEQ ID NO:6), or Arg-Cys-Ala-Phe-His-Leu-Gly-Glu-Leu-Val-Trp-Cys-Thr (SEQ ID NO:7).

In another embodiment, a polypeptide can include the amino acid sequence Cys-Ala-Xaa-His-Leu-Gly-Glu-Leu-Val-Trp-Cys (SEQ ID NO:8), in which Xaa can be Phe, Tyr, Trp, or Arg. For example, this document provides polypeptides that include the following amino acid sequences: Cys-Ala-Phe-His-Leu-Gly-Glu-Leu-Val-Trp-Cys-Thr (SEQ ID NO:9), Cys-Ala-Trp-His-Leu-Gly-Glu-Leu-Val-Trp-Cys-Thr (SEQ ID NO: 10), and Cys-Ala-Arg-His-Leu-Gly-Glu-Leu-Val-Trp-Cys-Thr (SEQ ID NO: 11).

The polypeptides provided herein can be modified for use in vivo by the addition, at the amino- or carboxy-terminal end, of a stabilizing agent to facilitate survival of the polypeptide in vivo. This can be useful in situations in which peptide termini tend to be degraded by proteases prior to cellular uptake. Such blocking agents can include, without limitation, additional related or unrelated peptide sequences that can be attached to the amino- and/or carboxy-terminal residues of the polypeptide (e.g., an acetyl group attached to the N-terminal amino acid or an amide group attached to the C-terminal amino acid). Such attachment can be achieved either chemically, during the synthesis of the polypeptide, or by recombinant DNA technology using methods familiar to those of ordinary skill in the art. Alternatively, blocking agents such as pyroglutamic acid or other molecules known in the art can be attached to the amino- and/or carboxy-terminal residues, or the amino group at the amino terminus or the carboxy group at the carboxy terminus can be replaced with a different moiety.

A proline or an Xaa-Pro-Pro (e.g., Ala-Pro-Pro) sequence at the amino terminus can be particularly useful (see, e.g., WO 00/22112). For example, a polypeptide can include the amino acid sequence Xaa₁-Pro-Pro-Cys-Ala-Xaa₂-His-Leu-Gly-Glu-Leu-Val-Trp-Cys-Thr (SEQ ID NO: 12), where Xaa₁ is any amino acid (e.g., Ala), and Xaa₂ is Trp, Tyr, Phe, or Arg. For example, a polypeptide can include the amino acid sequence Xaa-Pro-Pro-Cys-Ala-Trp-His-Leu-Gly-Glu-Leu-Val-Trp-Cys-Thr (SEQ ID NO: 13), Ala-Pro-Pro-Cys-Ala-Trp-His-Leu-Gly-Glu-Leu-Val-Trp-Cys-Thr (SEQ ID NO: 14), Xaa-Pro-Pro-Cys-Ala-Arg-His-Leu-Gly-Glu-Leu-Val-Trp-Cys-Thr (SEQ ID NO: 15), Ala-Pro-Pro-Cys-Ala-Arg-His-Leu-Gly-Glu-Leu-Val-Trp-Cys-Thr (SEQ ID NO: 16), Xaa-Pro-Pro-Cys-Ala-Phe-His-Leu-Gly-Glu-Leu-Val-Trp-Cys-Thr (SEQ ID NO: 17), or Ala-Pro-Pro-Cys-Ala-Phe-His-Leu-Gly-Glu-Leu-Val-Trp-Cys-Thr (SEQ ID NO:18). Alternatively, a polypeptide can include the amino acid sequence Xaa-Pro-Pro-Asp-Cys-Ala-Trp-His-Leu-Gly-Glu-Leu-Val-Trp-Cys-Thr (SEQ ID NO: 19), Ala-Pro-Pro-Asp-Cys-Ala-Trp-His-Leu-Gly-Glu-Leu-Val-Trp-Cys-Thr (SEQ ID NO:20), Xaa-Pro-Pro-Asp-Cys-Ala-Arg-His-Leu-Gly-Glu-Leu-Val-Trp-Cys-Thr (SEQ ID NO:21), Ala-Pro-Pro-Asp-Cys-Ala-Arg-His-Leu-Gly-Glu-Leu-Val-Trp-Cys-Thr (SEQ ID NO:22), Xaa-Pro-Pro-Asp-Cys-Ala-Phe-His-Leu-Gly-Glu-Leu-Val-Trp-Cys-Thr (SEQ ID NO:23), Ala-Pro-Pro-Asp-Cys-Ala-Phe-His-Leu-Gly-Glu-Leu-Val-Trp-Cys-Thr (SEQ ID NO:24), Xaa-Pro-Pro-Arg-Cys-Ala-Trp-His-Leu-Gly-Glu-Leu-Val-Trp-Cys-Thr (SEQ ID NO:25), Ala-Pro-Pro-Arg-Cys-Ala-Trp-His-Leu-Gly-Glu-Leu-Val-Trp-Cys-Thr (SEQ ID NO:26), Xaa-Pro-Pro-Arg-Cys-Ala-Arg-His-Leu-Gly-Glu-Leu-Val-Trp-Cys-Thr (SEQ ID NO:27), Ala-Pro-Pro-Arg-Cys-Ala-Arg-His-Leu-Gly-Glu-Leu-Val-Trp-Cys-Thr (SEQ ID NO:28), Xaa-Pro-Pro-Arg-Cys-Ala-Phe-His-Leu-Gly-Glu-Leu-Val-Trp-Cys-Thr (SEQ ID NO:29), or Ala-Pro-Pro-Arg-Cys-Ala-Phe-His-Leu-Gly-Glu-Leu-Val-Trp-Cys-Thr (SEQ ID NO:30), wherein Xaa at the first position is any amino acid.

The polypeptides provided herein can have a Pro-Pro-Xaa (e.g., Pro-Pro-Ala) sequence at their carboxy termini. For example, a polypeptide can include the amino acid sequence Cys-Ala-Trp-His-Leu-Gly-Glu-Leu-Val-Trp-Cys-Thr-Pro-Pro-Xaa (SEQ ID NO:31), Cys-Ala-Trp-His-Leu-Gly-Glu-Leu-Val-Trp-Cys-Thr-Pro-Pro-Ala (SEQ ID NO:32), Cys-Ala-Arg-His-Leu-Gly-Glu-Leu-Val-Trp-Cys-Thr-Pro-Pro-Xaa (SEQ ID NO:33), Cys-Ala-Arg-His-Leu-Gly-Glu-Leu-Val-Trp-Cys-Thr-Pro-Pro-Ala (SEQ ID NO:34), Cys-Ala-Phe-His-Leu-Gly-Glu-Leu-Val-Trp-Cys-Thr-Pro-Pro-Xaa (SEQ ID NO:35), Cys-Ala-Phe-His-Leu-Gly-Glu-Leu-Val-Trp-Cys-Thr-Pro-Pro-Ala (SEQ ID NO:36), Asp-Cys-Ala-Trp-His-Leu-Gly-Glu-Leu-Val-Trp-Cys-Thr-Pro-Pro-Xaa (SEQ ID NO:37), Asp-Cys-Ala-Trp-His-Leu-Gly-Glu-Leu-Val-Trp-Cys-Thr-Pro-Pro-Ala (SEQ ID NO:38), Asp-Cys-Ala-Arg-His-Leu-Gly-Glu-Leu-Val-Trp-Cys-Thr-Pro-Pro-Xaa (SEQ ID NO:39), Asp-Cys-Ala-Arg-His-Leu-Gly-Glu-Leu-Val-Trp-Cys-Thr-Pro-Pro-Ala (SEQ ID NO:40), Asp-Cys-Ala-Phe-His-Leu-Gly-Glu-Leu-Val-Trp-Cys-Thr-Pro-Pro-Xaa (SEQ ID NO:41), Asp-Cys-Ala-Phe-His-Leu-Gly-Glu-Leu-Val-Trp-Cys-Thr-Pro-Pro-Ala (SEQ ID NO:42), Arg-Cys-Ala-Trp-His-Leu-Gly-Glu-Leu-Val-Trp-Cys-Thr-Pro-Pro-Xaa (SEQ ID NO:43), Arg-Cys-Ala-Trp-His-Leu-Gly-Glu-Leu-Val-Trp-Cys-Thr-Pro-Pro-Ala (SEQ ID NO:44), Arg-Cys-Ala-Arg-His-Leu-Gly-Glu-Leu-Val-Trp-Cys-Thr-Pro-Pro-Xaa (SEQ ID NO:45), Arg-Cys-Ala-Arg-His-Leu-Gly-Glu-Leu-Val-Trp-Cys-Thr-Pro-Pro-Ala (SEQ ID NO:46), Arg-Cys-Ala-Phe-His-Leu-Gly-Glu-Leu-Val-Trp-Cys-Thr-Pro-Pro-Xaa (SEQ ID NO:47), or Arg-Cys-Ala-Phe-His-Leu-Gly-Glu-Leu-Val-Trp-Cys-Thr-Pro-Pro-Ala (SEQ ID NO:48), wherein Xaa can be any amino acid.

In some embodiments, the polypeptides provided herein can include additional amino acid sequences at the amino terminus of the sequence set forth in SEQ ID NO: 1, the carboxy terminus of the sequence set forth in SEQ ID NO: 1, or both. For example, a polypeptide can contain the amino acid sequence Trp-Glu-Ala-Xaa₁-Cys-Ala-Xaa₂-His-Xaa₃-Xaa₄-Xaa₅-Leu-Val-Trp-Cys-Xaa₆-Lys-Val-Glu-Glu (SEQ ID NO:49), wherein the residues denoted by Xaaₙ can display variability. As for the amino acid sequence set forth in SEQ ID NO:49, Xaa₁ can be absent or can be any amino acid (e.g., Arg or Asp); Xaa₂ can be Phe, Tyr, 5-HTP, Trp, or Arg; Xaa₃ can be any amino acid; Xaa₄ can be Gly or Ala; Xaa₅ can be Glu or Ala; and Xaa₆ can be absent or can be any amino acid (SEQ ID NO:58). In one embodiment, a polypeptide can include the amino acid sequence Trp-Glu-Ala-Asp-Cys-Ala-Xaa-His-Leu-Gly-Glu-Leu-Val-Trp-Cys-Thr-Lys-Val-Glu-Glu (SEQ ID NO:50), where Xaa is Arg, Trp, 5-HTP, Tyr, or Phe. For example, a polypeptide can include the amino acid sequence Trp-Glu-Ala-Asp-Cys-Ala-Trp-His-Leu-Gly-Glu-Leu-Val-Trp-Cys-Thr-Lys-Val-Glu-Glu (SEQ ID NO:51).

In another embodiment, a polypeptide can consist of the amino acid sequence (Xaa₁)ₘ-Xaa₂-Cys-Ala-Xaa₃-His-Xaa₄-Xaa₅-Xaa₆-Leu-Val-Trp-Cys-(Xaa₇)ₙ (SEQ ID NO:52), wherein the residues denoted by Xaa can display variability, and m and n can be, independently, integers from 0 to 10 (e.g., 0, 1, 2, 3, 4, 5, 6, 7, 8, 9, or 10). For example, Xaa₁ can be any amino acid; Xaa₂ can be absent or can be any amino acid (e.g., Arg or Asp); Xaa₃ can be Phe, Tyr, 5-HTP, Trp, or Arg; Xaa₄ can be any amino acid; Xaa₅ can be Gly or Ala; Xaa₆ can be Glu or Ala; Xaa₇ can be any amino acid; and m and n can be from 0 to 5 (e.g., 0, 1, 2, 3, 4, or 5) (SEQ ID NO:59). Alternatively, a polypeptide can consist of the amino acid sequence (Xaa₁)ₘ-Cys-Ala-Xaa₂-His-Leu-Gly-Glu-Leu-Val-Trp-Cys-Thr-(Xaa₃)ₙ(SEQ ID NO:53), wherein Xaa₁ is any amino acid, Xaa₂ is Phe or Arg, Xaa₃ is any amino acid, and m and n are, independently, integers from 0 to 5 (e.g., 0, 1, 2, 3, 4, or 5). Examples of polypeptides within these embodiments, without limitation, include polypeptides consisting of the amino acid sequence Ala-Ala-Ala-Ala-Ala-Asp-Cys-Ala-Arg-His-Leu-Gly-Glu-Leu-Val-Trp-Cys-Ala-A-la-Ala-Ala-Ala (SEQ ID NO:54), Ala-Ala-Arg-Cys-Ala-Arg-His-Leu-Gly-Glu-Leu-Val-Trp-Cys-Thr-Ala-Ala (SEQ ID NO:55), or Ala-Ala-Ala-Asp-Cys-Ala-Phe-Trp-His-Leu-Gly-Glu-Leu-Val-Trp-Cys-Thr-Ala-A-la (SEQ ID NO:56).

The amino acid sequences of the polypeptides described herein typically contain two cysteine residues. Polypeptides containing these amino acid sequences can cyclize due to formation of a disulfide bond between the two cysteine residues. A person having ordinary skill in the art can, for example, use Ellman's Reagent to determine whether a peptide containing multiple cysteine residues is cyclized. In some embodiments, these cysteine residues can be substituted with other natural or non-natural amino acid residues that can form lactam bonds rather than disulfide bonds. For example, one cysteine residue could be replaced with aspartic acid or glutamic acid, while the other could be replaced with ornithine or lysine. Any of these combinations could yield a lactam bridge. By varying the amino acids that form a lactam bridge, a polypeptide provided herein can be generated that contains a bridge approximately equal in length to the disulfide bond that would be formed if two cysteine residues were present in the polypeptide.

The polypeptides provided herein can contain an amino acid tag. A “tag” is generally a short amino acid sequence that provides a ready means of detection or purification through interactions with an antibody against the tag or through other compounds or molecules that recognize the tag. For example, tags such as c-myc, hemagglutinin, polyhistidine, or FLAG.® (an eight amino acid peptide tag; Sigma-Aldrich Corp., St. Louis, Mo.) can be used to aid purification and detection of a polypeptide. As an example, a polypeptide with a polyhistidine tag can be purified based on the affinity of histidine residues for nickel ions (e.g., on a Ni—NTA column), and can be detected in western blots by an antibody against polyhistidine (e.g., the Penta-His antibody; Qiagen, Valencia, Calif.). Tags can be inserted anywhere within the polypeptide sequence, although insertion at the amino- or carboxy-terminus is particularly useful.

The term “amino acid” refers to natural amino acids, unnatural amino acids, and amino acid analogs, all in their D and L stereoisomers if their structures so allow. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Unnatural amino acids include, but are not limited to 5-Hydroxytryptophan, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine, and pipecolic acid.

An “analog” is a chemical compound that is structurally similar to another but differs slightly in composition (as in the replacement of one atom by an atom of a different element or in the presence of a particular functional group). An “amino acid analog” therefore is structurally similar to a naturally occurring amino acid molecule as is typically found in native polypeptides, but differs in composition such that either the C-terminal carboxy group, the N-terminal amino group, or the side-chain functional group has been chemically modified to another functional group. Amino acid analogs include natural and unnatural amino acids which are chemically blocked, reversibly or irreversibly, or modified on their N-terminal amino group or their side-chain groups, and include, for example, methionine sulfoxide, methionine sulfone, S-(carboxymethyl)-cysteine, S-(carboxymethyl)-cysteine sulfoxide and S-(carboxymethyl)-cysteine sulfone. Amino acid analogs may be naturally occurring, or can be synthetically prepared. Non-limiting examples of amino acid analogs include 5-Hydroxytryptophan (5-HTP), aspartic acid-(beta-methyl ester), an analog of aspartic acid; N-ethylglycine, an analog of glycine; and alanine carboxamide, an analog of alanine Other examples of amino acids and amino acids analogs are listed in Gross and Meienhofer, The Peptides: Analysis, Synthesis, Biology, Academic Press, Inc., New York (1983).

The stereochemistry of a polypeptide can be described in terms of the topochemical arrangement of the side chains of the amino acid residues about the polypeptide backbone, which is defined by the peptide bonds between the amino acid residues and the α-carbon atoms of the bonded residues. In addition, polypeptide backbones have distinct termini and thus direction. The majority of naturally occurring amino acids are L-amino acids. Naturally occurring polypeptides are largely comprised of L-amino acids.

D-amino acids are the enantiomers of L-amino acids and can form peptides that are herein referred to as “inverso” polypeptides (i.e., peptides corresponding to native peptides but made up of D-amino acids rather than L-amino acids). A “retro” polypeptide is made up of L-amino acids, but has an amino acid sequence in which the amino acid residues are assembled in the opposite direction of the native peptide sequence.

“Retro-inverso” modification of naturally occurring polypeptides involves the synthetic assembly of amino acids with α-carbon stereochemistry opposite to that of the corresponding L-amino acids (i.e., D- or D-allo-amino acids), in reverse order with respect to the native polypeptide sequence. A retro-inverso analog thus has reversed termini and reversed direction of peptide bonds, while approximately maintaining the topology of the side chains as in the native peptide sequence. The term “native” refers to any sequence of L-amino acids used as a starting sequence for the preparation of partial or complete retro, inverso or retro-inverso analogs.

Partial retro-inverso polypeptide analogs are polypeptides in which only part of the sequence is reversed and replaced with enantiomeric amino acid residues. Since the retro-inverted portion of such an analog has reversed amino and carboxyl termini, the amino acid residues flanking the retro-inverted portion can be replaced by side-chain-analogous α-substituted geminal-diaminomethanes and malonates, respectively. Alternatively, a polypeptide can be a complete retro-inverso analog, in which the entire sequence is reversed and replaced with D-amino acids.

This document also provides peptidomimetic compounds that are designed on the basis of the amino acid sequences of polypeptides. Peptidomimetic compounds are synthetic, non-peptide compounds having a three-dimensional conformation (i.e., a “peptide motif,”) that is substantially the same as the three-dimensional conformation of a selected peptide, and can thus confer the same or similar function as the selected peptide. Peptidomimetic compounds can be designed to mimic any of the polypeptides provided herein.

Peptidomimetic compounds that are protease resistant are particularly useful. Furthermore, peptidomimetic compounds may have additional characteristics that enhance therapeutic utility, such as increased cell permeability and prolonged biological half-life. Such compounds typically have a backbone that is partially or completely non-peptide, but with side groups that are identical or similar to the side groups of the amino acid residues that occur in the peptide upon which the peptidomimetic compound is based. Several types of chemical bonds (e.g., ester, thioester, thioamide, retroamide, reduced carbonyl, dimethylene and ketomethylene) are known in the art to be useful substitutes for peptide bonds in the construction of peptidomimetic compounds.

The interactions between a polypeptide as described herein and an immunoglobulin molecule typically occur through the C_{H}2-C_{H}3 cleft of the immunoglobulin. Such interactions are engendered through physical proximity and are mediated by, for example, hydrophobic interactions. The “binding affinity” of a polypeptide for an immunoglobulin molecule refers to the strength of the interaction between the polypeptide and the immunoglobulin. Binding affinity typically is expressed as an equilibrium dissociation constant (K_{d}), which is calculated as K_{d}=k_{off}/kₒₙ, where k_{off}=the kinetic dissociation constant of the reaction, and kₒₙ=the kinetic association constant of the reaction. K_{d} is expressed as a concentration, with a low K_{d} value (e.g., less than 100 nM) signifying high affinity. Polypeptides that can interact with an immunoglobulin molecule typically have a binding affinity of at least 1 LM (e.g., at least 500 nM, at least 100 nM, at least 50 nM, or at least 10 nM) for the C_{H}2-C_{H}3 cleft of the immunoglobulin.

Polypeptides provided herein can bind with substantially equivalent affinity to immunoglobulin molecules that are bound by antigen and to monomeric immunoglobulins. Alternatively, the polypeptides described herein can have a higher binding affinity (e.g., at least 10-fold, at least 100-fold, or at least 1000-fold higher binding affinity) for immunoglobulin molecules that are bound by antigen than for monomeric immunoglobulins. Conformational changes that occur within the Fc region of an immunoglobulin molecule upon antigen binding to the Fab region are likely involved in a difference in affinity. The crystal structures of bound and unbound NC6.8 Fab (from a murine monoclonal antibody) showed that the tail of the Fab heavy chain was displaced by 19 angstroms in crystals of the antigen/antibody complex, as compared to its position in unbound Fab (Guddat et al. (1994) J. Mol. Biol. 236-247-274). Since the C-terminal tail of the Fab region is connected to the Fc region in an intact antibody, this shift would be expected to affect the conformation of the C_{H}2-C_{H}3 cleft. Furthermore, examination of several three-dimensional structures of intact immunoglobulins revealed a direct physical connection between the Fab heavy chain and the Fc C_{H}2-C_{H}3 (Harris et al. (1997) Biochemistry 36:1581-1597; Saphire et al. (2001) Science 293:1155-1159).

Molecular modeling of the C_{H}2-C_{H}3 cleft of monomeric (i.e., unbound) and immune-complexed IgG reveal that the monomeric Fc C_{H}2-C_{H}3 cleft has a closed configuration, which can prevent binding to critical amino acid residues (e.g., H is 435; see, for example, O'Brien et al. (1994) Arch. Biochem. Biophys. 310:25-31; Jefferies et al. (1984) Immunol. Lett. 7:191-194; and West et al. (2000) Biochemistry 39:9698-9708). Immune-complexed (antigen-bound) IgG, however, has a more open configuration and thus is more conducive to ligand binding. The binding affinity of RF for immune-complexed IgG, for example, is much greater than the binding affinity of RF for monomeric IgG (Corper et al. (1997) Nat. Struct. Biol. 4:374; Sohi et al. (1996) Immunol. 88:636). The same typically is true for the polypeptides provided herein.

Because the polypeptides described herein can bind to the C_{H}2-C_{H}3 cleft of immunoglobulin molecules, they can be useful for blocking the interaction of other factors (e.g., FcRn, FcR, C1q, histones, MBP, SOD1 and other immunoglobulins) to the Fc region of the immunoglobulin, and thus can inhibit Fc-mediated immune complex formation. By “inhibit” is meant that Fc-mediated immune complex formation is reduced in the presence of a polypeptide provided herein, as compared to the level of immune complex formation in the absence of the polypeptide. Such inhibiting can occur in vitro (e.g., in a test tube) or in vivo (e.g., in an individual). Any suitable method can be used to assess the level of immune complex formation. Many such methods are known in the art, and some of these are described herein.

The polypeptides described herein typically interact with the C_{H}2-C_{H}3 cleft of an immunoglobulin molecule in a monomeric fashion (i.e., interact with only one immunoglobulin molecule and thus do not link two or more immunoglobulin molecules together) with a 1:2 IgG Fc to peptide stoichiometry. Interactions with other immunoglobulin molecules through the Fc region therefore are precluded by the presence of the polypeptide. The inhibition of Fc-mediated immune complex formation can be assessed in vitro, for example, by incubating an IgG molecule with a labeled immunoglobulin molecule (e.g., a fluorescently or enzyme (ELISA) labeled Fc Receptor or C1q in the presence and absence of a polypeptide, and measuring the amount of labeled immunoglobulin that is incorporated into an immune complex. Other methods suitable for detecting immune complex formation also may be used, as discussed below.

### Preparation and Purification of Polypeptides

Polypeptides can be produced by a number of methods, many of which are well known in the art. By way of example and not limitation, a polypeptide can be obtained by extraction from a natural source (e.g., from isolated cells, tissues or bodily fluids), by expression of a recombinant nucleic acid encoding the polypeptide (as, for example, described below), or by chemical synthesis (e.g., by solid-phase synthesis or other methods well known in the art, including synthesis with an ABI peptide synthesizer; Applied Biosystems, Foster City, Calif.). Methods for synthesizing retro-inverso polypeptide analogs (Bonelli et al. (1984) Int. J. Peptide Protein Res. 24:553-556; and Verdini and Viscomi (1985) J. Chem. Soc. Perkin Trans. 1:697-701), and some processes for the solid-phase synthesis of partial retro-inverso peptide analogs also have been described (see, for example, European Patent number EP0097994).

This document provides isolated nucleic acid molecules encoding the polypeptides described herein. As used herein, “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand). The term “isolated” as used herein with reference to a nucleic acid refers to a naturally-occurring nucleic acid that is not immediately contiguous with both of the sequences with which it is immediately contiguous (one at the 5′ end and one at the 3′ end) in the naturally-occurring genome of the organism from which it is derived. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences that is normally immediately contiguous with the DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

This document also provides vectors containing the nucleic acids described herein. As used herein, a “vector” is a replicon, such as a plasmid, phage, or cosmid, into which another DNA segment may be inserted so as to bring about the replication of the inserted segment. Polypeptides can be developed using phage display, for example. Methods well known to those skilled in the art may use phage display to develop the polypeptides described herein. The vectors can be, for example, expression vectors in which the nucleotides encode the polypeptides provided herein with an initiator methionine, operably linked to expression control sequences. As used herein, “operably linked” means incorporated into a genetic construct so that expression control sequences effectively control expression of a coding sequence of interest. An “expression control sequence” is a DNA sequence that controls and regulates the transcription and translation of another DNA sequence, and an “expression vector” is a vector that includes expression control sequences, so that a relevant DNA segment incorporated into the vector is transcribed and translated. A coding sequence is “operably linked” and “under the control” of transcriptional and translational control sequences in a cell when RNA polymerase transcribes the coding sequence into mRNA, which then is translated into the protein encoded by the coding sequence.

Methods well known to those skilled in the art may be used to subclone isolated nucleic acid molecules encoding polypeptides of interest into expression vectors containing relevant coding sequences and appropriate transcriptional/translational control signals. See, for example, Sambrook et al., Molecular Cloning: A Laboratory Manual (2^{nd} edition), Cold Spring Harbor Laboratory, New York (1989); and Ausubel et al., Current Protocols in Molecular Biology, Green Publishing Associates and Wiley Interscience, New York (1989). Expression vectors can be used in a variety of systems (e.g., bacteria, yeast, insect cells, and mammalian cells), as described herein. Examples of suitable expression vectors include, without limitation, plasmids and viral vectors derived from, for example, herpes viruses, retroviruses, vaccinia viruses, adenoviruses, and adeno-associated viruses. A wide variety of suitable expression vectors and systems are commercially available, including the pET series of bacterial expression vectors (Novagen, Madison, Wis.), the Adeno-X expression system (Clontech), the Baculogold baculovirus expression system (BD Biosciences Pharmingen, San Diego, Calif.), and the pCMV-Tag vectors (Stratagene, La Jolla, Calif.).

Expression vectors that encode the polypeptides described herein can be used to produce the polypeptides. Expression systems that can be used for small or large scale production of polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules provided herein; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules provided herein; plant cell systems infected with recombinant virus expression vectors (e.g., tobacco mosaic virus) or transformed with recombinant plasmid expression vectors (e.g., Ti plasmid) containing the nucleic acid molecules provided herein; or mammalian cell systems (e.g., primary cells or immortalized cell lines such as COS cells, CHO cells, HeLa cells, HEK 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids provided herein.

The term “purified polypeptide” as used herein refers to a polypeptide that either has no naturally occurring counterpart (e.g., a peptidomimetic), or has been chemically synthesized and is thus uncontaminated by other polypeptides, or that has been separated or purified from other cellular components by which it is naturally accompanied (e.g., other cellular proteins, polynucleotides, or cellular components). Typically, the polypeptide is considered “purified” when it is at least 70%, by dry weight, free from the proteins and naturally occurring organic molecules with which it naturally associates. A preparation of purified polypeptide therefore can be, for example, at least 80%, at least 90%, or at least 99%, by dry weight, the polypeptide. Suitable methods for purifying polypeptides can include, for example, affinity chromatography, immunoprecipitation, size exclusion chromatography, and ion exchange chromatography. The extent of purification can be measured by any appropriate method, including but not limited to: column chromatography, polyacrylamide gel electrophoresis, or high-performance liquid chromatography.

### Methods of Modeling, Designing, and Identifying Compounds

This document provides methods for designing, modeling, and identifying compounds that can bind to the C_{H}2-C_{H}3 cleft of an immunoglobulin molecule and thus serve as inhibitors of Fc-mediated immune complex formation. Such compounds also are referred to herein as “ligands.” Compounds designed, modeled, and identified by these methods typically can interact with an immunoglobulin molecule through the C_{H}2-C_{H}3 cleft, and typically have a binding affinity of at least 1 μM (e.g., at least 500 nM, at least 100 nM, at least 50 nM, or at least 10 nM) for the C_{H}2-C_{H}3 cleft of the immunoglobulin. Such compounds generally have higher binding affinity (e.g., at least 10-fold, at least 100-fold, or at least 1000-fold higher binding affinity) for immune-complexed immunoglobulin molecules than for monomeric immunoglobulin molecules.

Compounds typically interact with the C_{H}2-C_{H}3 cleft of an immunoglobulin molecule in a monomeric fashion (i.e., interact with only one immunoglobulin molecule and thus do not link two or more immunoglobulin molecules together). The interactions between a compound and an immunoglobulin molecule typically involve the amino acid residues at positions 252, 253, 435, and 436 of the immunoglobulin (number according to Kabat, supra). For example, SEQ ID NO:20 (NB406) may have hydrophobic packing with IgG Fc Met-252, Ile-253, Ser-254, His-435 and Tyr-436 (e.g., the indole ring of Trp-14 in SEQ ID NO:20 can have a hydrophobic interaction with IgG Fc His-435). Alanine substitution of IgG Fc Asn-434, His-435 or Tyr-436 can disrupt binding (ΔΔG≧1.5 kcal/mol). In addition, alanine substitution of SEQ ID NO:20 Val-13 or Trp-14 can result in disruption of binding (ΔΔG≧2.0 kcal/mol).

The interaction between compounds and the C_{H}2-C_{H}3 cleft renders the compounds capable of inhibiting the Fc-mediated formation of immune complexes by blocking the binding of other factors (e.g., Fc:Fc interactions, FcγRs, FcRn, histones, MBP, MOG, RF, Tau protein, α-synuclein, SOD 1, TNF and C1q) to the C_{H}2-C_{H}3 cleft.

Compounds identified by the methods provided herein can be polypeptides such as, for example, those described herein. Alternatively, a compound can be any suitable type of molecule that can specifically bind to the C_{H}2-C_{H}3 cleft of an immunoglobulin molecule.

By “modeling” is meant quantitative and/or qualitative analysis of receptor-ligand structure/function based on three-dimensional structural information and receptor-ligand interaction models. This includes conventional numeric-based molecular dynamic and energy minimization models, interactive computer graphic models, modified molecular mechanics models, distance geometry and other structure-based constraint models. Modeling typically is performed using a computer and may be further optimized using known methods.

Methods of designing ligands that bind specifically (i.e., with high affinity) to the C_{H}2-C_{H}3 cleft of an immunoglobulin molecule having bound antigen typically are computer-based, and involve the use of a computer having a program capable of generating an atomic model. Computer programs that use X-ray crystallography data are particularly useful for designing ligands that can interact with an Fc C_{H}2-C_{H}3 cleft. Programs such as RasMol, for example, can be used to generate a three dimensional model of a C_{H}2-C_{H}3 cleft and/or determine the structures involved in ligand binding. Computer programs such as INSIGHT (Accelrys, Burlington, Mass.), GRASP (Anthony Nicholls, Columbia University), Dock (Molecular Design Institute, University of California at San Francisco), and Auto-Dock (Accelrys) allow for further manipulation and the ability to introduce new structures.

Methods can include, for example, providing to a computer the atomic structural coordinates for amino acid residues within the C_{H}2-C_{H}3 cleft (e.g., amino acid residues at positions 252, 253, 435, and 436 of the cleft) of an immunoglobulin molecule in an Fc-mediated immune complex, using the computer to generate an atomic model of the C_{H}2-C_{H}3 cleft, further providing the atomic structural coordinates of a candidate compound and generating an atomic model of the compound optimally positioned within the C_{H}2-C_{H}3 cleft, and identifying the candidate compound as a ligand of interest if the compound interacts with the amino acid residues at positions 252, 253, 435, and 436 of the cleft. The data provided to the computer also can include the atomic coordinates of amino acid residues at positions in addition to 252, 253, 435, and 436. By “optimally positioned” is meant positioned to optimize hydrophobic interactions between the candidate compound and the amino acid residues at positions 252, 253, 435, and 436 of the C_{H}2-C_{H}3 cleft.

Alternatively, a method for designing a ligand having specific binding affinity for the C_{H}2-C_{H}3 cleft of an immunoglobulin molecule can utilize a computer with an atomic model of the cleft stored in its memory. The atomic coordinates of a candidate compound then can be provided to the computer, and an atomic model of the candidate compound optimally positioned can be generated. As described herein, a candidate compound can be identified as a ligand having specific binding affinity for the C_{H}2-C_{H}3 cleft of an immunoglobulin molecule if, for example, the compound interacts with the amino acid residues at positions 252, 253, 435, and 436 of the cleft.

Such methods have shown that monomeric (non-antigen bound) IgG Fc binds at a site distinct from the IgG Fc C_{H}2-C_{H}3 cleft, such as the lower hinge region (Wines et al. (2000) J. Immunol. 164:5313-5318), while immune complexed (antigen bound) IgG Fc binding to FcγIIa is inhibited by an IgM rheumatoid factor (RF-AN), which has been shown by 3D structure to only bind to the IgG Fc C_{H}2-C_{H}3 interface cleft (Sohi et al. (1996) Immunology 88:636-641; and Corper et al. (1997) Nature Struct. Biol. 4(5):374-381). Soluble FcγIIa inhibits the binding of immune complexed (but not monomeric, non-immune complexed) IgG Fc to RF-AN (Wines et al. (2003) Immunol. 109:246-254), and inhibitors that bind to the IgG Fc C_{H}2-C_{H}3 cleft, such as the peptides described herein, inhibit the binding of immune complexed (antigen-bound) IgG Fc to FcγRs.

Compounds also can be interactively designed from structural information of the compounds described herein using other structure-based design/modeling techniques (see, e.g., Jackson (1997) Seminars in Oncology 24:L164-172; and Jones et al. (1996) J. Med. Chem. 39:904-917).

Compounds and polypeptides also can be identified by, for example, identifying candidate compounds by computer modeling as fitting spatially and preferentially (i.e., with high affinity) into the C_{H}2-C_{H}3 cleft of an immunoglobulin molecule, and then screening those compounds in vitro or in vivo for the ability to inhibit Fc-mediated immune complex formation. Suitable methods for such in vitro and in vivo screening include those described herein.

### Compositions and Articles of Manufacture

This document provides compositions and articles of manufacture that can be used in methods for treating conditions that arise from abnormal Fc-mediated immune complex formation (e.g., over-production of Fc-mediated immune complexes). The polypeptides, compounds, and compositions provided herein can be administered to a subject (e.g., a human or another mammal) having an viral infection, for example, that can be alleviated by modulating Fc-mediated immune complex formation and inhibit immune complexed IgG Fc to mC1q, sC1q, FcγRs, and FcRn. Typically, one or more polypeptides or compounds can be administered to a subject suspected of having a disease or condition associated with immune complex formation. Compositions generally contain one or more polypeptides and compounds described herein. A C_{H}2-C_{H}3 binding polypeptide, for example, can be in a pharmaceutically acceptable carrier or diluent, and can be administered in amounts and for periods of time that will vary depending upon the nature of the particular disease, its severity, and the subject's overall condition. Typically, the polypeptide is administered in an inhibitory amount (i.e., in an amount that is effective for inhibiting the production of immune complexes in the cells or tissues contacted by the polypeptide). The polypeptides and methods described herein also can be used prophylactically, e.g., to minimize immunoreactivity in a subject at risk for abnormal or over-production of immune complexes (e.g., a transplant recipient).

The ability of a polypeptide to inhibit Fc-mediated immune complex formation can be assessed by, for example, measuring immune complex levels in a subject before and after treatment. A number of methods can be used to measure immune complex levels in tissues or biological samples, including those that are well known in the art. If the subject is a research animal, for example, immune complex levels in the joints can be assessed by immunostaining following euthanasia. The effectiveness of an inhibitory polypeptide also can be assessed by direct methods such as measuring the level of circulating immune complexes in serum samples. Alternatively, indirect methods can be used to evaluate the effectiveness of polypeptides in live subjects. For example, reduced immune complex formation can be inferred from clinical improvement of immune mediated diseases or in vitro or in vivo models of which have been shown to be essential in the therapeutic use in treating Atherosclerosis.

Methods for formulating and subsequently administering therapeutic compositions are well known to those skilled in the art. Dosing is generally dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual polypeptides, and can generally be estimated based on EC50 found to be effective in in vitro and in vivo animal models. Typically, dosage is from 0.01 ug to 100 g per kg of body weight, and may be given once or more daily, biweekly, weekly, monthly, or even less often. Following successful treatment, it may be desirable to have the patient undergo maintenance therapy to prevent the recurrence of the disease state.

The present document provides pharmaceutical compositions and formulations that include the polypeptides and/or compounds described herein. Polypeptides therefore can be admixed, encapsulated, conjugated or otherwise associated with other molecules, molecular structures, or mixtures of compounds such as, for example, liposomes, polyethylene glycol, receptor targeted molecules, or oral, rectal, topical or other formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic compounds (e.g., C_{H}2-C_{H}3 binding polypeptides) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions can be administered by a number of methods, depending upon whether local or systemic treatment is desired and upon the area to be treated. Administration can be, for example, topical (e.g., transdermal, sublingual, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, C_{H}2-C_{H}3 binding polypeptides can be administered by injection or infusion into the cerebrospinal fluid, preferably with one or more agents capable of promoting penetration of the polypeptides across the blood-brain barrier.

Formulations for topical administration of C_{H}2-C_{H}3 binding polypeptides include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Nasal sprays are particularly useful, and can be administered by, for example, a nebulizer or another nasal spray device. Administration by an inhaler also is particularly useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including LIPOFECTIN® (a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in membrane-filtered water; Invitrogen/Life Technologies, Carlsbad, Calif.) and EFFECTENE™ (a non-liposomal lipid formulation in conjunction with a DNA-condensing enhancer; Qiagen, Valencia, Calif.).

Polypeptides can further encompass any pharmaceutically acceptable salts, esters, or salts of such esters, or any other compound which, upon administration to an animal including a human, is capable of providing (directly or indirectly) the biologically active metabolite or residue thereof. Accordingly, for example, this document provides pharmaceutically acceptable salts of polypeptides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the polypeptides provided herein (i.e., salts that retain the desired biological activity of the parent polypeptide without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); and salts formed with organic acids (e.g., acetic acid, citric acid, oxalic acid, palmitic acid, or fumaric acid).

Pharmaceutical compositions containing the polypeptides provided herein also can incorporate penetration enhancers that promote the efficient delivery of polypeptides to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas). Alternatively, inhibitory polypeptides can be delivered via iontophoresis, which involves a transdermal patch with an electrical charge to “drive” the polypeptide through the dermis.

Some embodiments provided herein include pharmaceutical compositions containing (a) one or more polypeptides and (b) one or more other agents that function by a different mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions. Other non-polypeptide agents (e.g., chemotherapeutic agents) also are within the scope of this document. Such combined compounds can be used together or sequentially.

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

The pharmaceutical formulations, which can be presented conveniently in unit dosage form, can be prepared according to conventional techniques well known in the pharmaceutical industry. Such techniques include the step of bringing into association the active ingredients (e.g., the C_{H}2-C_{H}3 binding polypeptides provided herein) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the polypeptide contained in the formulation.

The compositions described herein can be formulated into any of many possible dosage forms such as, but not limited to, tablets, capsules, liquid syrups, soft gels, suppositories, and enemas. The compositions also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

C_{H}2-C_{H}3 binding polypeptides can be combined with packaging material and sold as kits for reducing Fc-mediated immune complex formation. Components and methods for producing articles of manufacture are well known. The articles of manufacture may combine one or more of the polypeptides and compounds set out in the above sections. In addition, the article of manufacture further may include, for example, buffers or other control reagents for reducing or monitoring reduced immune complex formation. Instructions describing how the polypeptides are effective for reducing Fc-mediated immune complex formation can be included in such kits.

### Influenza Virus

The influenza virus causes influenza, which is more commonly known as the flu. Each year 3,000,000-5,000,000 severe cases of flu occur worldwide that results in 250,000-500,000 deaths, primarily in the very young, the very old or in people with health issues. Periodically, influenza pandemics occur that kill more than 1,000,000 people. During the 20^{th} century, three influenza pandemics have occurred, the Spanish influenza in 1918, the Asian influenza in 1958 and the Hong Kong influenza in 1968. The influenza A virus is the usual cause of flu and several different serotypes have been identified, including H1N1, which causes Spanish Flu and Swine Flu, H2N2, which causes Asian Flu, H3N2, which causes Hong Kong Flu and H5N1, which causes Bird Flu.

The influenza virus envelope contains two main glycoproteins, hemagglutinin and neuraminidase, which encapsulates 7-8 RNA segments, each of which encode one or two proteins. The influenza vaccine is highly effective; however, vaccinated people are still at risk of infection due to the highly variable serotypes of the virus and the inherent high mutation rate of the virus. Two different types of antivirals, the neuraminidase inhibitors, oseltamivir and zanavir, and the M2 proton channel protein inhibitors, amantadine and rimantadine, are available to treat patients with the flu, however, there are significant risks associated with the use of these antivirals. Given the great number of people that succumb to the flu each year and the resulting fatality rate, 5-17%, better treatment options for those patients with severe cases of flu are desperately needed.

A connection between the severity of flu and autoimmune disease has been observed in patients suffering from autoimmune diseases and is well documented (Blumentals et al., Musculoskelet Disord., 2012, 13:158; http://www.flu.gov/at-risk/health-conditions/arthritis/ and http://www.cdc.gov/arthritis/flu.htm). Moreover, some studies suggest that circulating immune complexes (ICs) could enhance the ability of the influenza virus to spread in infected individuals (Monsalvo et al., Nat Med., 2011, 17(2):195-9).

While most researchers argue that the greater susceptibility of patients with autoimmune diseases to influenza infections is due to their compromised health, unexpectedly, as described in Example 7, influenza virus contains Fc receptors (FcRs) that can bind to ICs. Thus, influenza virus could be using immune complexes as a mechanism to hijack the immune system and proliferate more rapidly. Moreover, as shown in Example 7, NB406 peptide prevents this binding from occurring and is expected to function as a therapeutic agent to prevent the proliferation or mitigate the severity of influenza virus infections.

### Hepatitis Virus

The hepatitis C virus infects the liver and can cause cirrhosis, liver failure and liver cancer. An estimated 150,000,000-200,000,000 people are estimated to be infected with hepatitis C worldwide and more than 500,000 hepatitis C related deaths occur each year. There is no vaccine for hepatitis C. The two best treatments for hepatitis C infections are Harvoni and Sovaldi, but they are incredibly expensive. Given the severity of hepatitis C viral infections and the expense of the current drugs that are available to treat it, a more reasonably priced drug is desperately needed. There are 6 different hepatitis virus that can cause liver infections, hepatitis A, B, C, D, E and F. While they are distinctly different viruses, their epidemiology and physiology are very similar.

An increased risk of hepatitis viral infections has been observed in patients suffering from autoimmune diseases. (Csepregi et al., J Rheumatol., 2001, 28:474-7; Ramos-Casals et al, Med Clin (Barc)., 2001, 19; 116(18):701-9). Some studies suggest that circulating immune complexes (ICs) could enhance the ability of hepatitis virus to spread in infected individuals (Araki et al, Acta Med Okayama., 1980, 34(2):131-8; Pfueller et al., Clin Exp Immunol., 1983, 54(3):655-60; Margolis et al., Hepatology, 1990, 11(1):31-7; Du et al., Zhonghua Zhong Liu Za Zhi, 2011 33(12):905-10)

While most researchers argue that the greater susceptibility of patients with autoimmune diseases to hepatitis viral infections is due to their compromised health, unexpectedly, as shown in Example 8, hepatitis A and C viruses contain Fc receptors (FcRs) that bind to ICs. Thus hepatitis virus could be using immune complexes as a mechanism to bridge (hijack) the immune system and proliferate more rapidly. Moreover, as shown in Example 8, the NB406 peptide can prevent this binding from occurring and thus can be expected to function as potential therapeutic agent to prevent the proliferation of hepatitis infections.

### Ebola Virus

Ebola or Ebola Virus Disease (EVD), and the more severe manifestation of EVD, Ebola hemorrhagic fever (EHF), are caused by infection with an Ebola virus strain. The Ebola virus causes an acute, febrile hemorrhagic illness which is often fatal if untreated. (Wiwanitkit, N. Am. J. Med. Sci., 2014, 6(11):549-552.) The Ebola virus can cause disease in humans and nonhuman primates (monkeys, gorillas, and chimpanzees). (CDC, About Ebola Virus Disease, available on the world wide web at cdc.gov/vhf/ebola/about.html) EVD first appeared in 1976 in two simultaneous outbreaks, one in Nzara, Sudan, and the other in Yambuku, Democratic Republic of Congo. The average EVD case fatality rate is around 50%. During outbreaks, case fatality rates have varied from 25% to 90%. (WHO Fact Sheet, Ebola Virus Disease, available on the world wide web at who.int/mediacentre/factsheets/fs103/en/#) As of January 2015, in the most recent outbreak, more than 20,000 cases of EVD had resulted in more than 8600 deaths. (CDC, 2014 Ebola Outbreak in West Africa—Case Counts, available on the world wide web at cdc.gov/vhf/ebola/outbreaks/2014-west-africa/case-counts.html)

There are five identified Ebola virus (EBOV) species, four of which are known to cause disease in humans: Ebola virus Zaire (Zaire ebolavirus; ZEBOV); Sudan virus (Sudan ebolavirus; SUDV or SEBOV); Taï Forest virus (Taï Forest ebolavirus; TAFV—formerly Côte d'Ivoire ebolavirus); and Bundibugyo virus (Bundibugyo ebolavirus; BDBV). The fifth, Reston virus (Reston ebolavirus; RESTV or REBOV), has been reported to cause disease in nonhuman primates and pigs, but not in humans. The natural reservoir host of Ebola virus and its means of transmission to humans from that host to humans remain unknown. (CDC, About Ebola Virus Disease, available on the world wide web at cdc.gov/vhf/ebola/about.html; Ansari et al., J. Autoimm., 2014, 55:1-9; Barrette et al., Science, 2009, 325(5937):204-206.)

Ebola virus and Marburg virus constitute the family Filoviridae in the order of Mononegvirales. Filoviruses are enveloped, non-segmented, negative-stranded RNA viruses. (Feldmann et al., Lancet, 2011, 377:849-62.) These filoviruses have characteristic filamentous particles that give the virus family its name.

### Ebola Virus Structure and Disease Progression

The Ebola virus genome consists of seven genes: nucleoprotein (NP), virion protein (VP) 35, VP40, glycoprotein (GP), VP30, VP24, RNA-dependent RNA polymerase (L). With the exception of the glycoprotein gene, all genes are monocistronic, encoding for one structural protein. (Feldmann et al., Lancet, 2011, 377:849-62.) The glycoprotein is the only transmembrane surface protein of the virus and forms trimeric spikes consisting of two subunits, glycoprotein 1 (GP1) and glycoprotein 2 (GP2), linked by a disulfide bond. (Feldmann et al., Lancet, 2011, 377:849-62; Jiang et al., Virol. Sin., 2009, 24(2): 121-135.) Ebola virus is distinguished from other Mononegavirales by its production of a soluble glycoprotein (sGP), which is the primary product of the GP gene, and gets secreted in large quantities from infected cells. (Feldmann et al., Lancet, 2011, 377:849-62.) The glycoprotein of Ebola virus mediates viral entry into host cells, augmentation of budding of viral particles, and interference with the expression of host cellular adhesion molecules. (Kuhl et al., J Infect. Dis., 2011, 204:S840-S849.) The characteristic filamentous shape of Ebola and Marburg viruses is largely determined by the matrix protein VP40. (Hartleib et al. Virology, 2006, 344:64-70.)

To date, the filovirus receptor on host cells has been elusive, but a variety of distinct and unrelated cell surface proteins have been implicated in viral entry including C-Type lectins, asialoglycoprotein receptor (ASGP-R), dendritic cell-specific ICAM-3 grabbin non-integrin (DC-SIGN), human macrophage galactose- and-acetylgalactosamine-specific C-type lectin (hMGL), and β1 integrin adhesion receptors. (Dolnik et al., Cell. Mol. Life Sci., 2008, 65:756-776; Barlbaud et al. Informa Healthcare, 2002 6(4):423 431.) Recently, the Niemann-Pick C1 (NPC1) receptor has been identified as an important mediator of Ebola infection of macrophages. (Dahlmann et al., J. Infect. Dis., published online Apr. 14, 2015.) None of the receptors identified to date explain the virus's ability to gain entry into diverse cell types and the resulting fast replication rate of Ebola virus in infected subjects.

Initially, Ebola virus primarily targets cells of the monocytes/macrophage and dendritic cell lineages; later in infection, the virus is able to gain entry into endothelial cells and hepatocytes. (Ansari et al., J. Autoimm., 2014, 55:1-9.) The virus is capable of replicating in a variety of cell types, including macrophages, epithelial cells, hepatocytes, and endothelial cells. (Gupta et al., J. Virol., 2004, 78(2):958-967.)

The detailed pathogenesis of Ebola virus infection is not well understood. In some cases, the infection can result in no symptoms or a mild infection characterized by transient flu like symptoms associated with mild coagulopathy, thrombocytopenia and leukocytosis, and full recovery. (Ansari et al., J. Autoimm., 2014, 55:1-9; Becquart et al., PLOS One, 2010, 5(2):e9126.) In other cases, the infection causes severe illness followed by hemorrhage, disseminated intravascular coagulation (DIC), shock, and death. The reasons for such varied outcomes are assumed to be secondary to the level of viral replication, with uncontrolled viral replication being associated with more serious illness. (Ansari et al., J. Autoimm., 2014, 55:1-9.)

In comparison to other hemorrhagic viral infections, patients having an Ebola virus infection have a higher viral load. Moreover, a higher viral load correlates with worse patient prognosis. For example, a recent study of 288 Ebola patients found that patients with a viral load of >10⁶ copies/ml had a higher case fatality rate than patients with <10⁶ copies/ml (Li et al., Int. J. Infect. Dis., 2016, 42:34-39 (published online Oct. 30, 2015)).

### Immune Complexes

Circulating immune complexes (CICs) have been shown to play a role in other viral infections, including dengue fever and dengue hemorrhagic fever (DHF). Immune complexes (IC) can form between anti-viral antibodies and viral antigens; between anti-viral antibodies and viral antigens from a different strain of the same virus; or between non-viral antibodies and non-viral antigens.

For example, during a second dengue infection with a different strain of the virus, dengue antibodies that bind but do not neutralize viral particles of the second strain of the virus are believed to bring circulating immune complexes into close proximity with the cell surface Fc receptors, which in turn facilitate viral entry into the cells, driving antibody-dependent enhancement (ADE) of dengue virus infection. (Boonak et al., J. Immunol., 2013, 190(11):5659-5665; Guzman et al., Arch Virol., 2013, 158(7):1445-1459; Chan et al., PNAS, 2011 108(30): 12479-12484.) Binding of dengue virus to non-dengue virus immune complexes has also been reported and suggested as a possible mechanism for ADE in DHF. (U.S. Pat. No. 8,815,813).

Additionally, in a subject infected with a first virus or disease, the immune response to a second, different virus or disease can include an antibody response, resulting in the formation of immune complexes between antibodies to the second virus or disease and antigens from the second virus or disease. In the case of an Ebola virus infection, an immune response to a second, different virus or disease could occur before or simultaneously with the Ebola virus infection. As used herein, the terms “heterologous immune complex” and “heterologous IC” mean a complex formed between a non-Ebola antibody and a non-Ebola antigen.

Reports have previously suggested that anti-Ebola virus immune complexes may enhance Ebola virus infection. (Johnson et al., Antiviral Res., 2014, 107:102-109; Takeda et al., J. Infect. Dis. 2007, 196:S347-56.) But Ebola virus disease has not been previously associated with heterologous immune complexes or circulating heterologous immune complexes. Moreover, the role of immune complexes in Ebola virus infection is controversial. Passive immunization, using antibodies produced in animals or sera/immunoglobulins from individuals who have survived infection has been used as a therapeutic vaccine post-infection. (Ansari et al., J. Autoimm., 2014, 55:1-9.) And use of an immune complex consisting of Ebola glycoprotein and a monoclonal antibody that recognizes an epitope in the glycoprotein has been suggested as an Ebola vaccine candidate. (Phoolcharoen et al., PNAS USA, 2011, 108(51):20695-700.) Such treatments could be ineffective (Oswald et al., PLOS Pathog., 2007, 3(1):e9) or even deleterious if immune complexes exacerbated Ebola virus disease.

### Novel Role for ICs and Viral FcRs in Ebola Virus Infections

Unexpectedly, as further described in the Examples, Ebola Virus and Ebola Virus glycoprotein (GP) can act as an Fc Receptor. Ebola Virus secretory glycoprotein (sGP) may also be able to act as an Fc Receptor. The viral Fc receptor (FcR) activity of Ebola virus could, therefore, permit binding of Ebola to an immune complex which is also bound to a host cell Fc receptor, enhancing entry of Ebola into host cells. That is, a heterologous immune complex, not just an anti-Ebola virus immune complex, could bridge a host cell-Ebola virus interaction. The results of Ebola virus GP binding to non-Ebola virus related immune complexes of peroxidase-rabbit anti-peroxidase IgG (“PAP-IC”) are shown in Example 5 below. These results demonstrate that Ebola GP may interact with heterologous immune complexes. Ebola's ability to interact with heterologous immune complexes may contribute to antibody dependent enhancement that accelerates disease progression, increasing Ebola virus infectivity and contributing to the pathogenicity of hemorrhagic fever and shock seen in Ebola virus infections.

The filamentous shape of Ebola virus and the number of Ebolaviral Fc receptor-functional glycoproteins differentiate it from other viruses expressing viral Fc-receptors, including Dengue virus. For example, a single Ebola virion may express on its surface thousands of trimeric glycoprotein spikes. (Tran et al. J. Virol., 2014, 88(18):10958-62; Beniac et al., PLOS One, 2012, 7(1): e29608.) In contrast, a Dengue virion has an icosahedral scaffold of just 90 glycoprotein E dimers. (Kuhn et al., Cell, 2002, 108(5):717-725.) Because an Ebola virion is may express many more viral Fc Receptor-functional surface proteins than a Dengue virion, Ebola may infect cells more quickly and replicate more quickly, increasing viral load.

The filamentous shape of Ebola virus may further enhance the ability of the glycoprotein of Ebola virus to act as a bridge. Because the glycoprotein binds to an IC which can also bind to cellular Fc receptors or complement receptors, a filamentous Ebola virion may attach to a host cell along the virion's length via a large number of interactions, promoting virus-host interaction, internalization, and/or infection. The long, filamentous shape of Ebola provides more points of contact with a host cell than the shorter, spherical or polyhedral shape of many other viruses including dengue.

In humans, co-infection with other diseases can cause an antibody response which results in the formation of heterologous immune complexes. Circulating immune complexes may cause antibody-dependent enhancement (ADE). The observed geographic correlation of serious malaria cases and the Ebola outbreak is consistent with the proposition that coinfection with other serious pathogenic agents may exacerbate Ebola virus infection. As described in Example 1, West Africa, the site of the unprecedented 2014 Ebola outbreak, represents a region having one of the highest numbers of serious Malaria cases (see Hay et al, PLOS Med., 2010, 7(6):e1000290). Also consistent with the exacerbation by coinfection proposition are the observations that some Ebola virus infections are asymptomatic (Becquart et al., PLOS One, 2010, 5(2):e9126), and that Ebola outbreaks have not occurred in other parts of the world, especially in the West where the incidence of co-infection other pathogenic agents is substantially lower. As described in Example 2, the observed incidence of porcine reproductive and respiratory syndrome virus (PRRSV) coinfection in REBOV-infected pig populations is likewise consistent with a proposed disease mechanism wherein REBOV replication is enhanced by coinfection, for example by immune complex (IC) formation induced by a non-related virus, PRRSV.

Exemplary diseases that can cause an antibody response resulting in the formation of heterologous immune complexes in humans include, for example, human immunodeficiency virus (HIV), human cytomegalovirus (HCMV), herpes simplex virus (HSV), hepatitis C virus, human papilloma virus (HPV), Mycobacterium tuberculosis, malaria/Plasmodium falciparum, and Schistosoma haematobium.

While many Fc receptors in humans are expressed only on immune cells including, for example, monocytes, macrophages, neutrophils, dendritic cells, mast cells, basophils, B cells, NK cells, NKT cells, etc., the neonatal Fc Receptor (FcRn) is expressed on both immune cells including, for example, antigen-presenting cells, monocytes/macrophages, neutrophils, and non-immune cells including, for example, vascular endothelial cells, and intestinal epithelial cells. (Bruhns, Blood, 2012, 119(24):5560-5649.) The viral Fc receptor of Ebola virus could, therefore, once bound to an IC, mediate entry into the cell types in which Ebola virus is known to replicate, including, for example, macrophages, epithelial cells, and endothelial cells. (Gupta et al., J. Virol., 2004, 78(2):958-967.) Polypeptides that bind to the C_{H}2-C_{H}3 cleft of an immunoglobulin molecule and inhibit Fc-mediated immune complex formation have been suggested as useful for treatment of ADE in dengue fever and dengue hemorrhagic fever. (U.S. Pat. No. 8,815,813.) Due to the unexpected finding that Ebola Virus and Ebola Virus glycoprotein (GP) can act as an Fc Receptor, such polypeptides may also affect interactions between Ebola Virus and ICs. Indeed, due to the fundamental differences in viral shape, in the number of surface receptors with Fc Receptor function, and in viral replication during disease progression, polypeptides that inhibit Fc-mediated immune complex formation may be significantly more effective when applied in the context of Ebola virus infections than for treatment of dengue virus infections.

Example 3 demonstrates that the binding of Ebola virus GP binding to heterologous immune complexes is inhibited by NB406 (SEQ ID NO:20; APPDCAWHLGELVWCT). In addition, SEQ ID NO:20 inhibited binding of either FcγRIIa or FcγRIIb to immune complexed IgG Fc by more than 70%, suggesting that SEQ ID NO:20 may abrogate antibody dependent enhancement (ADE) in Ebola virus disease caused by heterologous immune complexes (such as PAP-IC).

Methods for Using C_{H}2-C_{H}3 Binding Polypeptides to Inhibit Fc-Mediated Immune Complex Formation

C_{H}2-C_{H}3 binding polypeptides can be used in in vitro assays of Fc-mediated immune complex (IC) formation. Such methods are useful to, for example, evaluate the ability of a C_{H}2-C_{H}3 cleft-binding polypeptide to block Fc-mediated immune complex formation. In vitro methods can involve, for example, contacting an immunoglobulin molecule (e.g., an antigen bound immunoglobulin molecule) with an effector molecule (e.g., mC1q, sC1q, FcRs and FcRn, or another antibody) in the presence and absence of a polypeptide as provided herein, and determining the level of IC formation in each sample. Levels of IC formation can be evaluated by, for example, polyacrylamide gel electrophoresis with Coomassie blue or silver staining, or by co-immunoprecipitation. Such methods are known to those of ordinary skill in the art, and can be used to test the ability of a candidate polypeptide or compound to inhibit IC formation associated with an infectious disease, for example.

Methods provided herein also can be used to inhibit complement- or Fc-mediated immune complex formation in a subject, and to treat an infectious viral disease in a subject by inhibiting complement- or Fc-mediated immune complex formation. Such methods can include, for example, administering any of the polypeptides described herein, or a composition containing any of the polypeptides described herein, to a subject having or being at risk for having or developing an infectious viral disease (e.g., Ebola Virus Disease, influenza virus infection, hepatitis virus infection). For example, a method can include administering to an individual a composition containing a polypeptide that includes the amino acid sequence Cys-Ala-Trp-His-Leu-Gly-Glu-Leu-Val-Trp-Cys-Thr (SEQ ID NO: 10). Alternatively, a method can include administering to a subject a polypeptide that contains the amino acid sequence Asp-Cys-Ala-Trp-His-Leu-Gly-Glu-Leu-Val-Trp-Cys-Thr (SEQ ID NO:2), Xaa-Pro-Pro-Asp-Cys-Ala-Trp-His-Leu-Gly-Glu-Leu-Val-Trp-Cys-Thr (SEQ ID NO: 19; where Xaa is any amino acid), or Ala-Pro-Pro-Asp-Cys-Ala-Trp-His-Leu-Gly-Glu-Leu-Val-Trp-Cys-Thr (SEQ ID NO:20).

In some embodiments, when the viral disease is Ebola virus infection, a polypeptide can be administered to inhibit immune complex formation that is associated with antibody dependent enhancement (ADE), including complement-mediated antibody-dependent enhancement, of Ebola virus infection or contributes to the enhancement of an Ebola virus infection. The polypeptide can, for example, result in clinical or histiological improvement of an Ebola virus infection, result in an improvement or delay of onset of one or more of the histiological characteristics of an Ebola virus infection, and/or decrease the ADE of an Ebola virus infection. In some cases, the polypeptide can inhibit binding of a heterologous IC to an FcγR, inhibit formation of IC that contribute to immunopathogenesis of the ADE of EBOV infections, inhibit binding of EBOV virions to IgG IC, inhibit binding of a EBOV protein (e.g., GP) to an IgG immune complex, inhibit binding of EBOV and/or TNF-α to IgG IC, inhibit binding of a EBOV-IgG IC to FcγI, a FcγIIa H131 allele, a FcγIIa R131 allele, FcγRIIb, FcγRIIc, FcγRIIIa, FcγIIIb, or FcRn, and/or inhibit binding of EBOV-IgG IC to mC1q or sC1q.

In some embodiments, a polypeptide can be administered to a subject that has been diagnosed as having an Ebola virus infection, a subject suspected of having an Ebola virus infection, a subject exhibiting symptoms of an Ebola virus infection, a subject that is at risk of contracting an Ebola virus infection, and/or a subject that has been exposed to an Ebola virus. In some embodiments, a polypeptide can be administered to minimize the risk of a subject developing an Ebola virus infection. In one aspect, a polypeptide could be administered to a subject in a hospitalized population wherein one or more other subjects in the same hospitalized population has an Ebola virus infection, is suspected of having an Ebola virus infection, and/or is exhibiting symptoms of an Ebola virus infection.

In some embodiments, a polypeptide can be administered to a subject that has been diagnosed as having or is suspected of having a coinfection with a coinfective pathogen and has an Ebola virus infection, is suspected of having an Ebola virus infection, is exhibiting symptoms of an Ebola virus infection, is at risk of contracting an Ebola virus infection, and/or has been exposed to an Ebola virus. The coinfective pathogen may include, for example, human immunodeficiency virus (HIV), human cytomegalovirus (HCMV), herpes simplex virus (HSV), hepatitis C virus, human papilloma virus (HPV), Mycobacterium tuberculosis, malaria/Plasmodium falciparum, Schistosoma haematobium, and/or other disease associated with antibody production and/or immune complex formation. Such administration may be particularly helpful in co-infected individuals or individuals at risk of being co-infected because of the potential for non-Ebola virus related immune complexes to allow Ebola virus association with and entry into host cells.

In some embodiments, a polypeptide can be administered as soon as a subject is diagnosed with an Ebola virus infection, is suspected of having an Ebola virus infection, is exhibiting symptoms of an Ebola virus infection, is determined to be at risk of contracting an Ebola virus infection, and/or has been exposed to an Ebola virus. In some embodiments, a polypeptide can be administered to a subject who has or is suspected of having hemorrhagic fever resulting from an Ebola virus infection. In some embodiments, a polypeptide can be administered to a subject having a replicating Ebola virus. In some embodiments, a polypeptide can be administered to a subject in the later stages of Ebola virus infection or who is suspected of being in the advanced or later stages of Ebola virus infection or who is exhibiting hemorrhagic symptoms. In contrast to other viral infections, including, for example Dengue Fever infections and Dengue Hemorrhagic Fever, administration of a polypeptide to a subject in the advanced or later stages of Ebola virus infection, including to a subject having hemorrhagic fever, may still alleviate the symptoms and detrimental effects of Ebola virus infection because Ebola virus continues to replicate during the hemorrhagic fever stage and/or when a subject is exhibiting hemorrhagic symptoms.

In some embodiments, a polypeptide can be administered to a subject that has been diagnosed as having an influenza virus infection, a subject suspected of having an influenza virus infection, a subject exhibiting symptoms of an influenza virus infection, a subject that is at risk of contracting an influenza virus infection, and/or a subject that has been exposed to an influenza virus. In some embodiments, a polypeptide can be administered to minimize the risk of a subject developing an influenza virus infection. In one aspect, a polypeptide could be administered to a subject in a hospitalized population wherein one or more other subjects in the same hospitalized population has an influenza virus infection, is suspected of having an influenza virus infection, and/or is exhibiting symptoms of an influenza virus infection.

In some embodiments, a polypeptide can be administered as soon as a subject is diagnosed with an influenza virus infection, is suspected of having an influenza virus infection, is exhibiting symptoms of an influenza virus infection, is determined to be at risk of contracting an influenza virus infection, and/or has been exposed to an influenza virus.

In some embodiments, a polypeptide can be administered to a subject that has been diagnosed as having or is suspected of having an autoimmune disease and as having or is suspected of having an influenza virus infection, is suspected of having an influenza virus infection, is exhibiting symptoms of an influenza virus infection, is at risk of contracting an influenza virus infection, and/or has been exposed to an influenza virus. The autoimmune disease may include, for example, systemic lupus erythematosus, cryoglobulinemia, rheumatoid arthritis, scleroderma, Sjögren's syndrome, and/or another autoimmune disease associated with antibody production and/or immune complex formation. Such administration may be particularly helpful in individuals having an autoimmune disease because of the potential for non-virus related immune complexes to allow virus association with and entry into host cells.

In some embodiments, a polypeptide can be administered to a subject that has been diagnosed as having a hepatitis virus infection, a subject suspected of having a hepatitis virus infection, a subject exhibiting symptoms of a hepatitis virus infection, a subject that is at risk of contracting a hepatitis virus infection, and/or a subject that has been exposed to a hepatitis virus. In some embodiments, a polypeptide can be administered to minimize the risk of a subject developing a hepatitis virus infection.

In some embodiments, a polypeptide can be administered as soon as a subject is diagnosed with a hepatitis virus infection, is suspected of having a hepatitis virus infection, is exhibiting symptoms of a hepatitis virus infection, is determined to be at risk of contracting a hepatitis virus infection, and/or has been exposed to a hepatitis virus. In some embodiments, the hepatitis virus can include Hepatitis A and/or Hepatitis C.

In some embodiments, a polypeptide can be administered to a subject that has been diagnosed as having or is suspected of having an autoimmune disease and as having or is suspected of having a hepatitis virus infection, is suspected of having a hepatitis virus infection, is exhibiting symptoms of a hepatitis virus infection, is at risk of contracting a hepatitis virus infection, and/or has been exposed to a hepatitis virus. The autoimmune disease may include, for example, systemic lupus erythematosus, cryoglobulinemia, rheumatoid arthritis, scleroderma, Sjögren's syndrome, and/or another autoimmune disease associated with antibody production and/or immune complex formation. Such administration may be particularly helpful in individuals having an autoimmune disease because of the potential for non-virus related immune complexes to allow virus association with and entry into host cells.

In some embodiments, a polypeptide can be administered to a subject in combination with another suitable therapy. For example, in some embodiments, a polypeptide can be administered to a subject in combination with a composition comprising a therapeutic antibody. In some embodiments, a therapeutic antibody can include a monoclonal antibody to FcγRIIb including, for example, SuppreMol SM201 or Xencor XmAb5871. The composition comprising monoclonal antibody to FcγRIIb can be administered to a subject before, during, or after administration of a composition comprising the polypeptide.

In some embodiments, a polypeptide can be administered to a subject in combination with an composition including an antiretroviral including, for example, oseltamivir, zanamivir, peramivir, etc. The composition comprising the antiretroviral can be administered to a subject before, during, or after administration of a composition comprising the polypeptide.

In some embodiments, a polypeptide can be administered to a subject in combination with an composition including a drug approved for treating hepatitis including sofosbuvir and/or ledipasvir. The composition comprising the drug approved for treating hepatitis can be administered to a subject before, during, or after administration of a composition comprising the polypeptide.

Ebola is a filamentous and pleomorphic virus, attributes that are important in creating a “Velcro effect” possibly contributing to viral replication and immunopathogenicity. There have been five different viral sub-types that have been recognized: Ebolavirus Zaire (ZEBOV), Sudan Ebolavirus (SUDV or SEBOV), the Tai Forest Ebolavirus (TAFV), the Reston Ebolavirus (RESTV or REBOV) and the Bundibugyo Ebolavirus (BDBV). Each of these is pathogenic for humans except RESTV that so far has only been shown to be pathogenic for nonhuman primates (Journal of Autoimmunity xxx (2014) 1e9 Article in Press Ansari. Clinical features and pathobiology of Ebolavirus infection Review). Another filovirus, Lloviu virus (LLOV) appears to be pathogenic in bats (PLoS Pathog. 2011 7(10) e1002304 Negredo et al Discovery of an ebolavirus-like filovirus in Europe). Pathogenicity varies among Ebola viruses, from ZEBOV, which is highly lethal in humans, to REBOV, which causes disease in pigs and macaques but asymptomatically infects humans (Emerg Infect Dis. 2013 19(2) 270-3 Olival et al Ebola virus antibodies in fruit bats, Bangladesh).

Despite relatively a large population living in areas of risk and the widespread practice of bush-meat hunting in these predicted areas, Ebolavirus is rare both in suspected animal reservoirs and in terms of human outbreaks. It has been shown that the human population living within this niche is larger, more mobile and better internationally connected than when the pathogen was first observed. As a result, when spillover events do occur, the likelihood of continued spread amongst the human population is greater, particularly in areas with poor healthcare infrastructure. Whilst rare in comparison to other high burden diseases prevalent in this region, such as malaria, Ebola outbreaks can have a considerable economic and political impact, and the subsequent destabilization of basic health care provisioning in affected regions increases the toll of unrecorded morbidity and mortality of more common infectious diseases, throughout and after the epidemic period. The number of concurrent infections during the present outbreak represents a significant strain on healthcare systems that are already poorly provisioned (eLife 2014 3 e04395 Pigott et al Mapping the zoonotic niche of Ebola virus disease in Africa and references incorporated therein). No Ebola outbreaks were recorded prior to 1976, and the frequency of Ebola outbreaks has increased since 2000 (supra). At the same time, Globally, malaria cases and deaths grew rapidly from 1990 reaching a peak of 232 million cases (143 million to 387 million) in 2003 and 1.2 million deaths (1.1 million to 1.4 million) in 2004 (Murray et al Lancet 2014 384 9947 1005-1070 Global regional and national incidence and mortality for HIV tuberculosis and malaria during 1990 2013 a systematic analysis for the Global Burden Study 2013).

It has recently been shown that host genetics plays a very important role in the pathogenicity of EBOV infections, as a single strain of mouse adapted EBOV caused pathogenicity ranging from asymptomatic infections to fulminant infections closely resembling viral hemorrhagic fever, depending on the genetic makeup of the mice (Rasmussen et al Science Express 30 Oct. 2014 Page 1-10 Host genetic diversity enables Ebola hemorrhagic fever pathogenesis and resistance). Mice from the Collaborative Cross exhibit distinct disease phenotypes following mouse-adapted Ebola virus infection. Phenotypes range from complete resistance to lethal disease to severe hemorrhagic fever characterized by prolonged coagulation times and 100% mortality. Inflammatory signaling was associated with vascular permeability and endothelial activation, and resistance to lethal infection arose by induction of lymphocyte differentiation and cellular adhesion, likely mediated by the susceptibility allele Tek. These data indicate that genetic background determines susceptibility to Ebola hemorrhagic fever (supra).

These data suggest that EHF is characterized by earlier induction of a larger magnitude transcriptional response. In susceptible mice relative to resistant mice, genes associated with EBOV infection were differentially induced. Early in infection in the spleens of susceptible mice at day 1 p.i., we observed enrichment of p38 MAPK and ERK signaling processes that stimulate productive EBOV infection (supra). This observation suggests that activation of the immune system by p38 MARK and EKR should be conducive to EBOV replication, and a lack of immune activation by p38 MARK and EKR should contribute to abortive EBOV replication, possibly causing asymptomatic EBOV infections.

Consistent with this conclusion is the observation that the number of EBOV seropositive apparently asymptomatic individuals is much higher than previously thought (PLoS One. 2010 5(2) e9126 Becquart et al High prevalence of both humoral and cellular immunity to Zaire ebolavirus among rural populations in Gabon). The evidence that mild or asymptomatic EBOV infections occur can be found in several observations. First was the observations that in the 1976 SEBOV outbreak in Southern Sudan, the WHO found Ebola virus antibodies in 25 (19%) of 131 Maridi case-contacts and in 7 (19%) of 64 hospital staff contacts (that did not show any clinical signs of EBOV infection) indicates that Ebola virus can cause mild illness and even subclinical infections. . . . The antibody results with sera collected from the Nzara cotton factory indicate that 9 (37%) of the 24 staff . . . were infected . . . 7 (of the nine) gave no history of illness (WHO bulletin_1978_56(2)_247-270).

Further evidence of widespread asymptomatic Ebola infections has been found in a large Ebola seroprevalence study performed in Gabon. To better understand Zaire ebolavirus (ZEBOV) circulation and transmission to humans, Becquart et al. conducted a large serological survey of rural populations in Gabon, a country characterized by both epidemic and non-epidemic regions. The survey lasted three years and covered 4,349 individuals from 220 randomly selected villages, representing 10.7% of all villages in Gabon. Using a sensitive and specific ELISA method, Becquart et al. found a ZEBOV-specific IgG seroprevalence of 15.3% overall, the highest ever reported. The seroprevalence rate was significantly higher in forested areas (19.4%) than in other ecosystems, namely grassland (12.4%), savannah (10.5%), and lake land (2.7%). No other risk factors for seropositivity were found. The specificity of anti-ZEBOV IgG was confirmed by Western blot in 138 individuals, and CD8 T cells from seven IgG+ individuals were shown to produce IFN-c after ZEBOV stimulation. Together, these findings show that a large fraction of the human population living in forested areas of Gabon has both humoral and cellular immunity to ZEBOV (PLoS One. 2010 5(2) e9126 Becquart et al High prevalence of both humoral and cellular immunity to Zaire ebolavirus among rural populations in Gabon).

It is important to realize that the high mortality rate associated with Ebola Zaire may require other factors than simply exposure to an extremely pathogenic virus. It is generally accepted that ZEBOV is associated with a case fatality rate of about 90%, but this may be an overestimate. First, seven cases of asymptomatic infection were identified during the 1996 Booué outbreak in Gabon. Second, some ELISA-based serosurveys have shown high antibody prevalence rates among populations living in areas where no cases of EHF have ever been reported, suggesting that ZEBOV might also be capable of causing mild illness or even asymptomatic infection in humans. The IgG seroprevalence was 9.3% in villages located in the 1995 outbreak area around Kikwit, DRC, where no EHF cases were reported. Likewise, a seroprevalence of 13.2% was found in the Aka Pygmy population of Central African Republic, where no ZEBOV outbreaks have ever been reported (supra).

Certainly it can be argued that these numbers represent naturally immune individuals, however in the case of dengue viral infections (DENV), first infections without any evidence of antibody dependent enhancement of infection (ADE) rarely develop into Dengue Hemorrhagic Fever (DHF) and most cases of DHF involve ADE as part of the immunopathogenesis of DHF, possibility suggesting that ADE maybe a factor in the immunopathogenesis of other VHFs.

The other two examples of more widespread EBOV infections/prevalence than was previously thought come from the observations of Ebola Reston (REBOV) infection in domestic swine in both the Philippines and China co-infected with porcine reproductive and respiratory syndrome virus (PRRSV) demonstrating REBOV in Asia and the report that ZEBOV and Marburg virus (MARV) has been found in fruit bats in Bangladesh (Olival et al. Emerg Infect Dis. 2013 19(2) 270-3, Olival et al, Ebola virus antibodies in fruit bats, Bangladesh). To determine geographic range for Ebola virus, Olival et al. tested 276 bats in Bangladesh. Five (3.5%) bats were positive for antibodies against Ebola Zaire and Reston viruses; no virus was detected by PCR. These bats might be a reservoir for Ebola or Ebola-like viruses, and extend the range of filoviruses to mainland Asia (supra).

The presence of high seroprevalence rate in very geographically diverse areas and serological evidence of past EBOV mild or symptomatic infections suggests that EBOV is much more prevalent than thought and the occurrence of widespread EHF may involve factors other than simply exposure to a highly virulent pathogen (supra).

Another key to understanding the immunopathogenesis of EBOV infection is the observation that B cell knock-out (KO) mice, which express no antibodies, have partial immunity and develop persistent Ebola infections. Using a mouse model, Gupta et al. determined the role of the immune system in clearance of and protection against Ebola virus. All CD8 T-cell-deficient mice succumbed to subcutaneous infection and had high viral antigen titers in tissues, whereas mice deficient in B cells or CD4 T cells cleared infection and survived, suggesting that CD8 T cells, independent of CD4 T cells and antibodies, are critical to protection against subcutaneous Ebola virus infection. B-cell-deficient mice that survived the primary subcutaneous infection (vaccinated mice) transiently depleted or not depleted of CD4 T cells also survived lethal intraperitoneal rechallenge for >25 days. However, all vaccinated B-cell-deficient mice depleted of CD8 T cells had high viral antigen titers in tissues following intraperitoneal rechallenge and died within 6 days, suggesting that memory CD8 T cells by themselves can protect mice from early death. Surprisingly, vaccinated B-cell-deficient mice, after initially clearing the infection, were found to have viral antigens in tissues later (day 120 to 150 post-intraperitoneal infection). Furthermore, following intraperitoneal rechallenge, vaccinated B-cell-deficient mice that were transiently depleted of CD4 T cells had high levels of viral antigen in tissues earlier (days 50 to 70) than vaccinated undepleted mice. This demonstrates that under certain immunodeficiency conditions, Ebola virus can persist and that loss of primed CD4 T cells accelerates the course of persistent infections. These data show that CD8 T cells play an important role in protection against acute disease, while both CD4 T cells and antibodies are required for long-term protection, and they provide evidence of persistent infection by Ebola virus suggesting that under certain conditions of immunodeficiency a host can harbor virus for prolonged periods, potentially acting as a reservoir (J Virol 2004 78 2 958-967 Gupta et al Persistent Infection with Ebola Virus under Conditions of Partial Immunity).

Previous reports suggest that one of two outcomes occurs with Ebola virus infection in humans and nonhuman primates: either the virus produces an overwhelming infection that rapidly leads to death of the host, or it is cleared by a vigorous immune response that results in complete recovery of the host. Thus, Ebola hemorrhagic fever is characteristically an acute illness, and the outcome usually becomes apparent fairly early in the course of infection; a prolonged course of infection has not been reported (supra).

The virus replicates in a variety of cell types, including macrophages, epithelial cells, hepatocytes, and endothelial cells. Viral replication in these cells induces high levels of inflammatory chemokines and cytokines that may be responsible for the inflammatory pathology observed during the early phase of infection. However, alpha/beta interferon responses, which would normally inhibit viral replication and dissemination, are suppressed by the virus. Binding of the Ebola virus viral glycoprotein to endothelial cells induces cytotoxic effects and increases vascular permeability, which may provide a mechanism for endothelial cell leakage in later stages of infection. Evidence from human studies suggests that a poor cellular immune response (as measured by low levels of gamma interferon and CD8 T-cell activation markers in serum) and low levels of anti-Ebola virus immunoglobulin G are associated with fatal infections. In contrast, convalescent patients have high levels of anti-Ebola virus immunoglobulin G and typically clear the infection within 3 weeks after onset of symptoms. Studies in animal models suggest that the presence of high titers of antibodies can protect the host from fatal infection. However, neither the mechanisms of protection during natural infection nor the elements of the immune response that are responsible for viral clearance are well understood (supra).

A mouse model of Ebola virus infection has been developed for use in studying immune responses to the virus in an attempt to understand the correlates of protective immunity. In this model, subcutaneous infection with an adapted Ebola virus results in a nonfatal infection associated with long term immunity against lethal rechallenge. Using this model, we show that CD8 T cells play a crucial role in the initial clearance of the virus following primary and secondary (rechallenge) infections and that CD4 T cells and antibodies are not required for short-term protection. However, in the absence of B cells and antigen-specific CD4 T cells, Ebola virus establishes a persistent asymptomatic infection, with disease symptoms appearing only during the late stage of infection. These data indicate that short-term control of the virus is achieved by CD8 T cells alone, but long-term control requires the presence of antibodies and CD4 T cells (supra).

Passive transfer of high-titer polyclonal antibodies from vaccinated Ebola virus-immune cynomolgus macaques to naive macaques failed to confer protection against disease, suggesting a limited role of humoral immunity. In contrast, depletion of CD8(+) T cells in vivo after vaccination and immediately before challenge eliminated immunity in two vaccinated macaques, indicating a crucial requirement for T cells in this setting. The protective effect was mediated largely by CD8(+) cells, as depletion of CD8(+) cells in vivo using the cM-T807 monoclonal antibody (mAb), which does not affect CD4(+) T cell or humoral immune responses, abrogated protection in four out of five subjects (Nat Med. 2011 17(9) 1128-31 Sullivan et at CD8+ cellular immunity mediates rAd5 vaccine protection against Ebola virus infection of nonhuman primates).

Consistent with the observation that activation of mitogen-activated protein kinase (MAPK) is conducive to Ebola viral replication is the finding that inhibitors of p38 MAPK can abort fatal Ebola infections: Antigen presenting cells (APCs), including macrophages and dendritic cells, are early and sustained targets of Ebola virus (EBOV) infection in vivo. EBOV activates mitogen-activated protein kinase (MAPK) signaling upon infection of APCs. Pyridinyl imidazole inhibitors of p38 MAPK on EBOV infection of human APCs and EBOV mediated cytokine production from human DCs. The p38 MAPK inhibitors reduced viral replication in PMA-differentiated macrophage-like human THP-1 cells and primary human monocyte-derived dendritic cells (MDDCs) and cytokine production from EBOV-treated MDDCs was inhibited in a dose-dependent manner (Antiviral Res 2014 107 102-9 Johnson et al Pyridinyl imidazole inhibitors of p38 MAP kinase impair viral entry and reduce cytokine induction by Zaire ebolavirus in human dendritic cells). These results suggest that activation of p38 MAPK and ERK is necessary for productive EBOV infection. Since activation of macrophages/DC in the presence of immune complexes (IC) results in the activation of p38 MAPK and ERK, EBOV IC would be expected to enhance EBOV infection (supra).

It is important to realize that IC by themselves do not trigger activation of the immune system, rather activation of MAPK requires both IC and an antigenic stimulus:

Strong et al. reported a model system in which infection of mouse and bat cell lines with EBOV leads to persistence, which can be broken with low levels of lipopolysaccharide or phorbol-12-myristate-13-acetate (PMA). This reactivation depends on the Ras/MAPK pathway through inhibition of RNA-dependent protein kinase and eukaryotic initiation factor 2α phosphorylation and occurs at the level of protein synthesis. EBOV also can be evoked from mice 7 days after infection by PMA treatment, indicating that a similar mechanism occurs in vivo. Our findings suggest that EBOV may persist in nature through subclinical infection of a reservoir species, such as bats, and that appropriate physiological stimulation may result in increased replication and transmission to new hosts. Identification of a presumptive mechanism responsible for EBOV emergence from its reservoir underscores the “hit-and-run” nature of the initiation of human and/or nonhuman primate EBOV outbreaks (PNAS 2008 105(46) 17982-7 Strong et al Stimulation of Ebola virus production from persistent infection through activation of the Ras-MAPK pathway).

An obvious trigger for productive EBOV replication in bats is blood parasites belonging to Haemosporida: Bats are the hosts to a large array of blood parasites (e.g. Bartonella, piroplasms, trypanosomes and microfillaria). In particular, bats are host to a unique collection of Haemosporida (Apicomplexa). Besides Plasmodium spp. and the rarer Hepatocystis spp., which infect several orders of Mammalia, bats host at least two unique genera not found outside the Chiroptera, Nycteria and Polychromophilus, plus two additional genera known from a single record (Dionisia and Biguetellia). The diversity of haemosporidian parasites of bats suggests, together with a recent phylogeny, that the Chiroptera might have been the original host of all mammalian Haemosporida (Parasit Vectors. 2014 7(1) 566 Witsenburg et al Epidemiological traits of the malaria-like parasite Polychromophilus murinus in the Daubenton's bat Myotis daubentonii). If both weaker condition and younger age are associated with higher infection intensity, then the highest selection pressure exerted by P. murinus should be at the juvenile stage (supra).

This finding is consistent with the seasonal pulses of Marburg in bats: Marburg virus, like its close relative Ebola virus, can cause large outbreaks of hemorrhagic fever with case fatalities nearing 90%. For decades the identity of the natural reservoir was unknown. However, in 2007 Marburg viruses were isolated directly from Egyptian fruit bats (Rousettus aegyptiacus) that inhabited a Ugandan gold mine where miners were previously infected. Soon after, two tourists became infected with Marburg virus after visiting nearby Python Cave, a popular attraction in Queen Elizabeth National Park, Uganda. This cave also contained R. aegyptiacus bats (40,000 animals). These events prompted a long-term investigation of Python Cave to determine if, 1) R. aegyptiacus in the cave carried infectious Marburg virus genetically similar to that found in the tourists, and 2) what ecological factors might influence virus spillover to humans. In the study, we found that, 1) approximately 2.5% of the bat colony is actively infected at any one time and that virus isolates from bats are genetically similar to those from infected tourists, and 2) specific age groups of bats (juveniles, six months of age) are particularly likely to be infected at specific times of the year that roughly coincide with historical dates of Marburg virus spillover into humans (Author summary, Amman et al PLos Pathogens October 2012 Volume 8 Issue 10 e1002877 Seasonal Pulses of Marburg Virus Circulation in Juvenile Rousettus aegyptiacus Bats Coincide with Periods of Increased Risk of Human Infection).

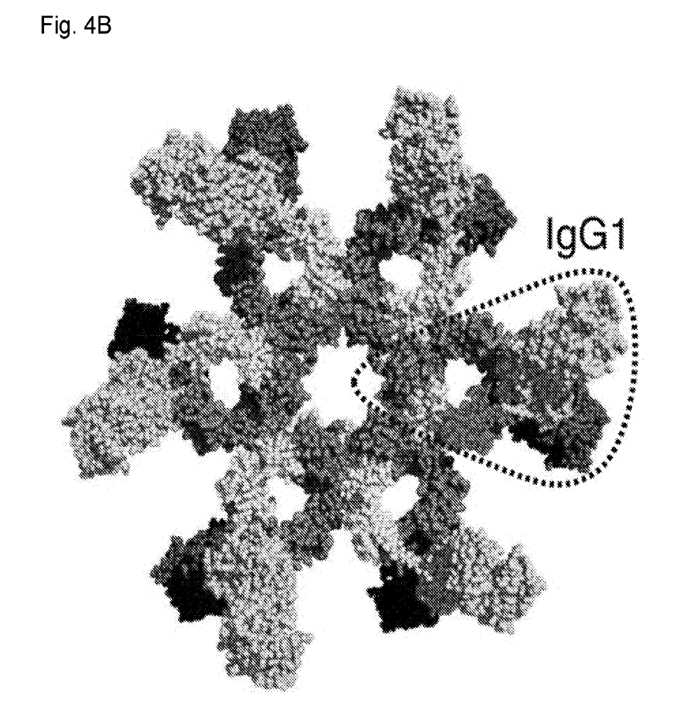

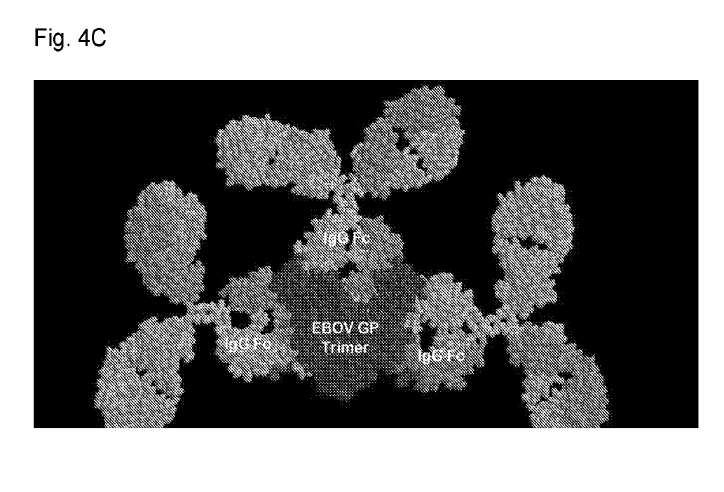

The approximate dates of 13 suspected Marburg virus spillover events were determined from the literature. . . . When all 13 Marburg virus spillover events are listed by month of occurrence, the data show a temporal clustering of human infections, coinciding with the summer (mid-June through mid-September) and winter months (mid-December through mid-March) of the northern hemisphere. The majority of spillover events (7/13) involved resident African miners, suggesting that the clustering effect was not due to seasonal tourism. More importantly, when the dates of these 13 spillover events are compared to a sinusoidal curve derived from the field collection data showing the seasonal incidence of juvenile R. aegyptiacus infections (FIG. 4), a pattern of coincidence emerges. The sinusoidal curve has peaks and troughs that correspond to the beginning of the birthing and breeding seasons respectively, each separated by roughly three months, and whose peak heights reflect the average percentage of infected juveniles for each seasonal category. These data show that 11 of 13 (84.6%, Fisher's Exact Test p, 0.05) spillover events occurred during the three month periods encompassing each of the two biannual birthing seasons when juvenile bats are roughly 4.5-7.5 months old and most likely to be infected with Marburg virus. Moreover, when suspected (extrapolated) exposure dates for 52 primary cases (all miners and epidemiologically unlinked to any other human cases; Table S1) from the final MHF patient list from the 1998-2000 outbreak in Durba, DRC are included in the analysis (Pierre Rollin and Robert Swanepoel; personal communication; Table S2), 54 of 65 (83.1% Fisher's Exact Test p, 0.05) spillover events occur during the same periods encompassing each of the biannual birthing seasons, further supporting the idea that these three-month periods may represent times of increased risk for exposure to Marburg virus. The contribution of young naive bats to the overall population during these seasons is considerable (supra).

Ebola outbreaks in humans also have a similar seasonality, Ebola outbreaks in great apes have always been reported at the beginning of the dry seasons (December 1995 in Mayibout, July 1996 in Booué, July 2001 in Mekambo, December 2001 in Kelle, and December 2002 in the second Kelle outbreak). Thus, Ebola outbreaks probably do not occur as a single outbreak spreading throughout the Congo basin as others have proposed, but are due to multiple episodic infection of great apes from the reservoir (Science 2004 303 5656 387-390 Leroy et al Multiple Ebola virus transmission events and rapid decline of central African wildlife).

Ebola infections are characterized by high levels of IL-10 (Journal of Autoimmunity xxx (2014) 1e9 Article in Press Ansari. Clinical features and pathobiology of Ebolavirus infection Review), suggesting that an immune mechanism that stimulates high levels of IL-10 would be conducive to Ebola virus replication:

Activation of macrophages in the presence of immune complexes increases the production of IL-10 and reduces IL-12 production (J Immunol. 2005 175(1) 469-77 Lucas et al ERK activation following macrophage FcγR ligation leads to chromatin modifications at the IL-10 locus). Enhanced production of IL-10 correlates with a rapid and enhanced activation of two MAPKs, ERK and p38 (supra). We feel that this response to FcγR ligation is a universal response that is a general property of most, if not all, macrophages. In the present work we sought to determine the mechanism whereby IL-10 was induced in response to immune complexes. Immune complexes alone were not sufficient to induce IL-10. Rather cytokine production required both the stimulation (such as LPS) and the addition of immune complexes (IC). Only the combination of these two stimuli resulted in high levels of IL-10 production (supra). This observation suggests that a pathogen that increases circulating immune complexes and presents an antigenic stimulus may favor EBOV replication and therefore be a cofactor for Filoviruses via ADE.

Since it has been suggested that activation of the immune system by p38 MARK and EKR should be conducive to EBOV replication (Rasmussen et al Science Express 2014 1 10 1126 Host genetic diversity enables Ebola hemorrhagic fever pathogenesis and resistance), the effect of high circulating IC, or more importantly, the presence of antigenic stimulus and IC in REBOV/PRRSV co-infected pigs may be conducive to REBOV infection in pigs and serves as an in vivo example of an Ebola cofactor, wherein the cofactor facilitates Ebola viral replication and infection.

An in vivo example of such an enhancement due to co-infection with PRRSV can be found in REBOV infection in pigs. An example of a concurrent viral infection as a possible cofactor for Ebola infectivity can be found in Ebola Reston infection of pigs in both the Philippines and China. The only instances of clinical (not experimental) Ebola Reston (REBOV) infection in domestic swine in both the Philippines and China have been when the pigs were also co-infected with porcine reproductive and respiratory syndrome virus (PRRSV) that were experiencing a severe respiratory disease syndrome (Science 2009 325(5937) 204-6 Barrette et al Discovery of swine as a host for the Reston ebolavirus Arch Virol. 2014 159(5) 1129-32, Pan et al Reston virus in domestic pigs in China). Since there have been no clinical reports of REBOV infection without co-infection of swine with PRRSV it appears that the co-infection REBOV with PRRSV maybe an obligate part of clinical REBOV infection in pigs.

Polyclonal B cell activation and hypergammaglobulinemia occur in association with various viral infections that stimulate type I IFNs that in turn enhance B cell proliferation. This hypergammaglobulinemia is often associated with autoimmunity perhaps because of nonspecific proliferation of B cells expressing a preimmune-like repertoire that is known to be polyreactive and to recognize autoantigens. Although previous spectratypic analyses show that B cell CDR3s from PRRSV infected piglets were a signature for polyclonal activation, certain HCDR3 lengths were pronounced (JI 2007 178 6320-6331 Bulter et al Antibody Repertoire Development in Fetal Cells with Hydrophobic HCDR3s and Neonatal Piglets-XIX. Undiversified B Antibody Repertoire Development in Fetal Cells Preferentially Proliferate in the PRRSV and references incorporated therein).

Data presented herein show that so-called polyclonal B cell activation in this viral infection is instead selective expansion of B cells with hydrophobic HCDR3s that are normally deselected during development to yield an oligoclonal population with hydrophobic HCDR3s. This biased expansion means that the normally dominant, neutral-charged HCDR3 population seen in newborns and other treatment groups is poorly represented in the V_{H} repertoire, consistent with immune dysregulation in this disease. This effect appears to be due to selective proliferation of naive B cells of the preimmune repertoire that do not diversify We assume that the Igs responsible for the hypergammaglobulinemia in PRRSV-infected piglets result from the B cells with hydrophobic HCDR3s that we report here. The alternative is that the secreted Igs are not derived from the predominant B cells but rather from a minor population within the hydropathicity spectrum (supra).

There are few clues to explain B cell selection during PRRSV infection and no published evidence that PRRSV infects B cells. Viral epitopes are generally hydrophilic glycoproteins, but amphipathic helices that can be hydrophobic are present in ORF proteins including PRRSV (supra).

The polyclonal expansion of B cells by PRRSV is a classic example of a B cell superantigen: The fact that B cells recognize Ag differently than T cells-soluble Ag versus MHC class I1-associated peptides-suggests that Ig-SAg will structurally and functionally differ from T-SAg. Nevertheless, we can use the T-SAg as a model to predict the minimum requirements for an Ig-SAg. First, an Ig-SAg would be expected to bind primarily to the F(ab) region of the Ig molecule (i.e., VH-DH-JH and/or VL-JL). Second, an Ig-SAg would bind to a family of Ig based on a common sequence and/or tertiary structure of the F(ab). This would predict that analogous to a T-SAg an Ig-SAg would bind to a substantially larger B cell population than a conventional Ag (Am J Pathol. 1994 144(4) 623-36 Goodglick et al Revenge of the microbes: Superantigens of the T and B cell lineage). The first example of an Ig-SAg is a phage-encoded membrane protein of S. aureus, protein A. In addition to the well characterized binding site of protein A on the Fc portion of IgG molecules, there is an independent binding site for protein A in the F(ab) region of a subfraction of Ig molecules. Surprisingly, the alternate binding site of protein A was found to be specific for VH3 family Ig. VH3 is the largest VH family consisting of 30 or more members. Binding of protein A to VH3 was independent of isotype (heavy chain constant region) or light chain usage (supra).

S. aureus Cowan (SAC) is a well-known mitogen and differentiative agent for B cells, and protein A is a necessary component of this activity. Recent studies have demonstrated that SAC and purified protein A specifically activate VH3-expressing human B cells. A second example of a putative Ig-SAg is the outer envelope glycoprotein of the HIV, gp120 (supra, Int Immunol. 2000 12(3) 305-12 Neshat et al Mapping the B cell superantigen binding site for HIV-1 gp120 on a V(H)3 Ig). This protein is responsible for the binding of HIV to CD4 on the surface of T cells and other CD4-expressing cells and is therefore critical to the infection of CD4 cells. An important and poorly explained feature of HIV infection is the striking polyclonal activation of B lymphocytes in this disease, resulting in clinical manifestations including lymphoid hyperplasia, hypergammaglobulinemia, autoantibody-mediated autoimmune disorders, and in late infection a dramatically increased frequency of B cell lymphoma. HIV gp120 binds to a subpopulation (4 to 8%) of normal B cells from uninfected, seronegative individuals. This subpopulation of B cells did not express CD4, but rather bound gp120 via membrane Ig of the VH3 gene family. The direct interaction of gp120 with VH3 was confirmed by cell-free binding studies of gp120 with purified VH3 antibodies. Functionally, gp120 served as a surrogate Ag for the in vitro activation and Ig secretion by VH3 B cells. In clinical HIV infection, this interaction is correlated with selective expansion of the VH3 B cells and serum VH3 IgM (in healthy seropositive individuals), followed by a profound VH3 B cell deletion (in individuals with AIDS). This array of findings strongly implicates HIV gp120 as an Ig-SAg for the VH3 Ig family (supra). Since HIV infection creates similar immunological conditions as PRRSV, HIV is very likely a co-factor accelerating EBOV replication and pathogenicity.

It is also important to realize that anti-SIV gp120 antibodies can cross react with VH3 immunoglobulins, which has to be taken into account when designing a vaccine for Ebola (Scand J Immunol. 2003 57(3) 239-45 Zigment-Reed et al Cross-reaction of anti-simian immunodeficiency virus envelope protein antibodies with human immunoglobulins). This is important in that any EBOV vaccine candidate that stimulates autoimmunity will likely fail to protect against actual Ebola infections.

Another example of a viral expansion of self-reactive V_{H} CDR3s is found in HCV infections. Analysis of the immunoglobulin receptor (IGR) variable heavy- and light-chain sequences on 17 hepatitis C virus (HCV)-associated non-Hodgkin lymphomas (NHLs) (9 patients also had type II mixed cryoglobulinaemia [MC] syndrome and 8 had NHL unrelated to MC) and analysis of intraclonal diversity on 8 of them suggest that such malignant lymphoproliferations derive from an antigen-driven pathologic process, with a selective pressure for the maintenance of a functional IgR and a negative pressure for additional amino acid mutations in the framework regions (FRs). For almost all NHLs, both heavy- and light-chain complementarity-determining regions (CDR3) showed the highest similarity to antibodies with rheumatoid factor (RF) activity that have been found in the MC syndrome, thus suggesting that a common antigenic stimulus is involved in MC syndrome and in HCV-associated lymphomagenesis. Moreover, because HCV is the recognized pathologic agent of MC and the CDR3 amino acid sequences of some HCV-associated NHLs also present a high homology for antibody specific for the E2 protein of HCV, it may be reasonable to speculate that HCV E2 protein is one of the chronic antigenic stimuli involved in the lymphomagenetic process. Finally, the use of specific segments, in particular the D segment, in assembling the IgH chain of IgR seems to confer B-cell disorders with the property to produce antibody with RF activity, which may contribute to the manifestation of an overt MC syndrome (Blood. 2000 96(10) 3578-84 De Re et al Sequence analysis of the immunoglobulin antigen receptor of HCV-associated non-Hodgkin lymphomas suggests that the malignant cells are derived from the RF-producing cells and type II cryoglobulinea). The presence of both IC and superantigen activity in HCV infections suggests that HCV may be a cofactor in Ebola infections.

It is important to note that HCV core protein also expresses a viral FcγR, capable of binding any IC: Maillard et al. have previously demonstrated that viral particles with the properties of nonenveloped hepatitis C virus (HCV) nucleocapsids occur in the serum of HCV-infected individuals. We show here that nucleocapsids purified directly from serum or isolated from HCV virions have FcγR-like activity and bind nonimmune IgG via its Fcγ domain. HCV core proteins produced in Escherichia coli and in the baculovirus expression system also bound nonimmune IgG and their Fcγ fragments. Folded conformation was required for IgG binding because the FcγR-like site of the core protein was inactive in denaturing conditions. Studies with synthetic core peptides showed that the region spanning amino acids 3-75 was essential for formation of the IgG-binding site. The interaction between the HCV core and human IgG is more efficient in acidic (pH 6.0) than in neutral conditions. The core protein-binding site on the IgG molecule differs from those for C1q, FcγRII (CD32), and FcγRIII (CD16) but overlaps with that for soluble protein A from Staphylococcus aureus (SpA), which is located in the CH2-CH3 interface of IgG. These characteristics of the core-IgG interaction are very similar to those of the neonatal FcRn.

Surface plasmon resonance studies suggested that the binding of an anti-core antibody to HCV core protein might be bipolar through its paratope to the corresponding epitope and by its Fcγ region to the FcγR-like motif on this protein. These features of HCV nucleocapsids and HCV core protein may confer an advantage for HCV in terms of survival by interfering with host defense mechanisms mediated by the Fcγ part of IgG (J Biol Chem. 2004 279(4) 2430-7 Maillard et al Fc gamma receptor-like activity of hepatitis C virus core protein).

The observation that REBOV replication appears to be enhanced in the presence of hydrophobic IC, similar to the immunopathology of HCV infections/MC syndrome, where the immune response is not simply diverted away from the Ebola virus, but directed towards self-antigens, particularly those with RF specificity, has important ramifications in understanding Ebola immunopathogenesis. An analysis of immunoglobulin binding proteins from a variety of pathogens suggests that the use of hydrophobic amino acid anchor residues present on a viral FcγR are likely to be directed towards the IgG Fc CHγ2-CHγ3 cleft may be a common denominator in the immunopathogenesis of these pathogens.

A similar immunopathology of superantigen, IC and viral FcγR/immunoglobulin binding protein can be found in herpes viral infections: A T cell superantigen and selective activation of certain Plasma Cells by the Viral M2 Superantigen has been described for herpes virus (Plos One 2014 10 8 e1004302 O'Flaherty et al The Murine Gammaherpesvirus Immediate-Early Rta Synergizes with IRF4, Targeting Expression of the Viral M1 Superantigen to Plasma Cells).

Based on the analysis of M2 function, we propose that M2 falls into a new class of herpesvirus genes that do not directly impact virus replication, but rather facilitate virus reactivation from latency by manipulating cellular differentiation/activation leading to a reactivation competent cellular environment. We have adopted the term reactivation conditioner for such genes. In the case of viruses that establish latency in memory lymphocytes, it is attractive to speculate that it may be necessary to encode functions that drive quiescent memory B or T cells into a state which is more conducive to virus replication. With respect to latency established in memory B cells, plasma cells would appear particularly well suited to support herpesvirus replication. As such, we hypothesize that manipulation of plasma cell differentiation leading to virus reactivation from latently infected memory B cells is relevant to reactivation of the human gammaherpesviruses. Although there is no obvious M2 homolog in either EBV or KSHV, there are several well documented examples of conserved functions encoded by gammaherpesvirus latency-associate gene products that lack obvious sequence homology. Indeed, our previous observation that M2 expression in primary murine B cells triggers IL-6 and IL-10 expression, recapitulates functions modulated by both EBV and KSHV, and provides further evidence of pathogenic strategies that are conserved among this family of viruses (supra).

Herpes Simplex Virus 1 (HSV-1) infects 40-80% of adults worldwide. HSV-1 initiates infection at mucosal surfaces and spreads along sensory neurons to establish a life-long latent infection that can lead to neurological diseases. Humans usually develop IgG antibodies that specifically recognize pathogens via fragment antigen binding (Fab) variable regions. HSV-1 can avoid the protective effects of antibodies by producing gE-gI, a receptor that binds to the constant portion of IgGs (Fc), thereby tethering the antibody in a position where it cannot trigger downstream immune functions. A gE-gI-bound IgG can participate in antibody bipolar bridging (ABB) such that the Fabs bind a viral antigen and the Fc binds gE-gI. The fate of ABB complexes had been unknown. We used live cell fluorescent imaging to follow ABB complexes during their formation and transport within a cell. We demonstrated that ABB assemblies were internalized into acidic intracellular compartments, where gE-gI dissociated from IgG-viral antigen complexes and the IgG and antigen were targeted for degradation within lysosomes. These results suggest that gE-gI mediates clearance of infected cell surfaces of both anti-viral IgGs and viral antigens, a general mechanism to facilitate latent infection by evading IgG mediated responses (Ndjamen et al PLoS Pathogens 2014 10 3 e1003961 the Herpes Virus Fc Receptor gE gl mediates Antibody Bipolar Bridging to Clear Viral Antigens from the Cell Surface).

Herpes Simplex Virus (HSV), Varicella-Zoster Virus (VZV), and Pseudorabies Virus (PrV) are members of the alpha herpes virus family, which are characterized by a relatively short replicative cycle in epithelial tissues and egression to and latent infection of the sensory neurons. Alpha herpes viruses have evolved many strategies to evade the host immune system. For example, antibodies do not appear to function effectively in clearance of HSV-1. It has been shown that the severity and persistence of HSV-1 lesions do not correlate with serum levels of neutralizing antibodies in infected individuals. HSV-1 encodes type 1 transmembrane glycoproteins, glycoprotein E (gE) and glycoprotein I (gI), that are displayed on the surface of infected cells and virions. Together they function as a receptor for the Fc region of human immunoglobulin G (IgG) and have also been implicated in cell-to-cell spread of virus. In addition, gE is required for HSV-1 movement inside both neuronal and epithelial cells. The Fc receptor function of gE-gI, which hinders access to the IgG Fc region and thus allows HSV-infected cells to escape recognition by Fc-dependent effector cells, may serve as a mechanism to block antibody-related host defenses (Ndjamen et al PLoS Pathogens 2014 10 3 e1003961 the Herpes Virus Fc Receptor gE gl mediates Antibody Bipolar Bridging to Clear Viral Antigens from the Cell Surface and references incorporated therein).

Antibody bipolar bridging (ABB), in which an anti-viral IgG bound to a cell surface antigen also binds to an Fc receptor, has the potential to protect virions and infected cells from IgG mediated immune responses. gE-gI is an HSV-1 heterodimeric complex that can function as a receptor for human IgGs by binding to their Fc regions, thus it can mediate ABB in HSV-1-infected cells. Experiments performed in HSV-1-infected cells to compare the efficacy of IgGs that can or cannot form ABB complexes suggested that bipolar bridging protects HSV-1 and HSV-1-infected cells from antibody- and complement-dependent neutralization, antibody-dependent cell-mediated cytotoxicity, and granulocyte attachment (supra). These observations suggest that the presence of a superantigen, IC and a viral FcγR/IBP found in herpes viral infections would be conducive to Ebola viral replication and that herpes viral infections may be cofactors for Ebola infections.

Similar to Ebola, B cell deficient mice are resistant to Malaria infections and developing cerebral Malaria, an important aspect of serious Malaria contributing to the morbidity and mortality of Malaria infected individuals. B-cell-deficient mice, devoid of immunoglobulins, exhibited increased survival and delayed onset of disease. Histopathology revealed striking differences, with a lower degree of microvascular hemorrhage in the B-cell-deficient mice (M Bio. 2014 5(2) e00949-14 Oliveria et al Increased survival in B-cell-deficient mice during experimental cerebral malaria suggests a role for circulating immune complexes).

ICs have proinflammatory properties, and indeed, studies currently being carried out by Oliveria et al have shown that human peripheral blood mononuclear cells stimulated with ICs isolated from the serum of individuals with Plasmodium vivax malaria secrete interleukin-6, tumor necrosis factor, and IL-1 (D. Golenbock and R. Gazzinelli, unpublished observations). The loss of CR1 that Oliveria et al proposed coincides with a rise in IgG- and IgM containing ICs and may represent an attempt to clear these proinflammatory complexes from the circulation (supra). Altogether, we propose that CIC plays a major role in the pathogenesis of cerebral Malaria and that immunoglobulin deficiency alters the disease outcome following P. berghei ANKA infection (supra).

Complement receptor 1 (CR1) expressed on the surface of phagocytic cells binds complement bound IC (C3b opsonized) playing an important role in the clearance of circulating immune complexes (IC). This receptor is critical to prevent accumulation of IC, which can contribute to inflammatory pathology.

Accumulation of circulating IC is frequently observed during malaria, although the factors contributing to this accumulation are not clearly understood. Oliveria et al observed that the surface expression of CR1 on monocyte/macrophages and B cells is strongly reduced in mice infected with Plasmodium yoelii, a rodent malaria model. Monocyte/macrophages from these infected mice present a specific inhibition of complement-mediated internalization of IC caused by the decreased CR1 expression. Accordingly, mice show accumulation of circulating IC and deposition of IC in the kidneys that inversely correlates with the decrease in CR1 surface expression. The results reported by Oliveria et al indicate that malaria induces a significant decrease on surface CR1 expression in the monocyte/macrophage population that results in deficient internalization of IC by monocyte/macrophages. To determine whether this phenomenon is found in human malaria patients, Oliveria et al analyzed 92 patients infected with either P. falciparum or P. vivax, the most prevalent human malaria parasites. The levels of surface CR1 on peripheral monocyte/macrophages and B cells of these patients show a significant decrease compared to uninfected control individuals in the same area. Oliveria et al proposed that this decrease in CR1 plays an essential role in impaired IC clearance during malaria (supra).

TCR Vβ usage was examined in C57BL/6 mice infected with Plasmodium yoelii. In addition to a polyclonal T cell activation, already described, a superantigenic-like activity was observed during the acute infection. This superantigenic activity induces a preferential deletion without prior expansion of CD4⁺ and CD8⁺ T cells bearing the TCR Vβ9 segment. The superantigen could be released by the parasite at different stages of its development since the deletion of Vβ9⁺ T cells was observed in blood and lymph nodes of mice infected either with sporozoites or with erythrocytic stages. Injection of sporozoite or parasitized erythrocytes to newborn mice led to a deletion and anergy of peripheral Vβ9⁺ T cells, without affecting thymic T cell populations. These observations suggest that the superantigen is released at very low concentrations during parasite development (International Immunology 1996 9 117-125 Pied et al Evidence for superantigenic activity during murine malaria infection).

Profound perturbations of the immune system are observed during the infection such as (i) hypergammaglobulinemia, with a lack of plasmodial specificity resulting from a polyclonal B cell activation, and (ii) induction of hyporesponsiveness of T cells to plasmodial and non-plasmodial antigens, and perturbation of the CD4/CD8 ratio. The cause of this phenomenon is not known, nevertheless there is evidence of a defect in both production of IL-2 and expression of IL-2 receptor by peripheral blood lymphocytes in response to stimulation with malaria-specific antigen during acute Plasmodium falciparum malaria in humans). It has been suggested that the strong polyclonal activation of all lymphocyte populations observed during malaria infection is due to a mitogen released by Plasmodium (supra).

In general, superantigens from a number of pathogens stimulate T lymphocytes through particular TCR Vβ chains. The recognition of T cells depends almost exclusively on the Vβ domain and, consequently, a superantigen can interact with a large fraction of the T cell repertoire, because the number of Vβ genes is low. In contrast to conventional antigens, superantigens bind specifically with a region of TCR located on the Vβ chain, outside of the specific site which combines with the MHC-peptide complex. When superantigens are encountered during T cell development they usually induce a clonal deletion, or an anergy, of T cells bearing the appropriate Vβ. In order to identify T cell populations and define the T cell repertoire involved in the host response to malaria infection, we have studied TCR Vβ chain usage in different cell compartments of C57BL/6 mice infected either with sporozoites or erythrocytic stages of Plasmodium. We describe here superantigenic-like activity associated with the parasite (supra).

The in vivo response to bacterial toxins usually results in preferential deletion, or anergy, in the responding CD4⁺ subpopulation; although some exceptions have been observed where both CD4⁺ and CD8⁺ subsets are affected. The infection with exogenous Murine Mammary Tumor Virus is dominated by deletion or anergy in the responding CD4⁺ lymphocytes. In the murine model of Chagas' disease, the in vivo Trypanosoma cruzi superantigenic effect was observed in the CD8 compartment. In a second parasitic system which concerns Toxoplasma gondi, the in vitro response to a superantigen is restricted to CD8⁺ lymphocytes. By contrast, the P. yoelii superantigenic activity affects both Vβ9⁺ CD4⁺ and CD8⁺ subsets. Again, by analogy with the effects obtained in the chronic exposure to low dose of SEA (staphylococcal enterotoxin A—discussed in reference) which results in the disappearance of CD4⁺ and CD8⁺ T cells bearing Vβ3, we can hypothesize that the plasmodial superantigen is expressed at low levels during the time of the infection (supra).

Aberrant immune activation induced by chronic infections with Plasmodium falciparum: leads to polyclonal B cell activation characterized by the presence of hyperglobulinemia, elevated titers of autoantibodies, and frequent occurrence of Burkitt's lymphoma and splenic lymphoma. The mechanisms that lead to this polyclonal B cell activation are poorly understood. The marked effect of malaria infection on B cells is related both to the biology of the infection, and to the nature of the malarial Ags. P. falciparum-infected erythrocytes (IE) have the potential to directly interact with B cells in different anatomical sites and to induce B cell proliferation and differentiation into Ab-secreting cells. We have shown that a large proportion (83%) of fresh isolates of IE bind nonimmune Igs, suggesting that in the peripheral blood IE could interact with B cells through their surface Igs. Moreover, blood borne antigens (and thus malarial Ags related to the erythrocytic phase) are trapped in the spleen where B cells represent about 40% of the splenocytes. Thus, IE and their constituent Ags could interact in the spleen with B cells displaying a variety of surface phenotypes, Ag-binding repertoires and signaling profiles. Among malarial Ags, the P. falciparum erythrocyte membrane protein 1 (PfEMP1) family of proteins often display Ig binding properties. The Ig binding activity of the PfEMP1, cloned from two different P. falciparum strains, resides in two different variable domains, the Duffy binding-like domain 2β (DBL2β) and the cysteine-rich interdomain region 1α (CIDR1α). The latter domain has been identified as a polyclonal B cell activator and an Ig binding protein (IBP) with a binding pattern similar to that of another microbial IBP, the protein A of Staphylococcus aureus. Microbial IBPs are produced by protozoa, viruses, and bacteria, and play important physiological roles. During an infectious process, IBPs may act as an evasion mechanism to divert specific Ab responses. CIDR1α binds to and activates purified B lymphocytes in vitro, an interaction partially mediated through the binding to surface Ig (J Immunol. 2006 177(5) 3035-44 Donati et al Increased B cell survival and preferential activation of the memory compartment by a malaria polyclonal B cell activator).

Up-regulated genes induced by CIDR1α includes several genes previously described as mitogen-activated, involved in pathways that control cell growth/apoptosis, transcription/translation, and that are normally induced during immune responses. The up-regulation of both TRAF3 and TRAF4 suggests activation of the NF-_{K}B pathway. The increased expression of two of the most important protein kinases, PKC and the PKA related forms (PRKAG3, PRKX), is in line with the observed CIDR1α-mediated up-regulation of genes involved in different signaling pathways; among them: the MEK/ERK pathway (EIF2B5), the MEKK/JNK pathway (TRAF3, GPS1), and the MKK/MAPK pathways (MAPK4K5, IQGAP1, IQGAP2) (supra).

Antigen presenting cells (APCs), including macrophages and dendritic cells, are early and sustained targets of Ebola virus (EBOV) infection in vivo. EBOV activates mitogen-activated protein kinase (MAPK) signaling upon infection of APCs. Pyridinyl imidazole inhibitors of p38 MAPK on EBOV infection of human APCs and EBOV mediated cytokine production from human DCs. The p38 MAPK inhibitors reduced viral replication in PMA-differentiated macrophage-like human THP-1 cells and primary human monocyte-derived dendritic cells (MDDCs) and cytokine production from EBOV-treated MDDCs was inhibited in a dose-dependent manner (Antiviral Res 2014 107 102-9 Johnson et al Pyridinyl imidazole inhibitors of p38 MAP kinase impair viral entry and reduce cytokine induction by Zaire ebolavirus in human dendritic cells). These results suggest that activation of p38 MAPK and ERK is necessary for productive EBOV infection. Since activation of macrophages/DC in the presence of immune complexes (IC) results in the activation of p38 MAPK and ERK, EBOV IC would be expected to enhance EBOV infection (supra).

Since productive EBOV infection is dependent on the activation of p38 MAPK and ERK, this suggests that Malaria can act as a cofactor accelerating EBOV replication. Evidence exists for superantigen activity in EBOV infections, the archetype B cell superantigen is Staph Protein A (SpA):

Superantigen activity has also been described for Ebola: human ZEBOV infection is associated with mRNA downregulation of three TCR Vβ subsets, indicating either anergy or deletion of the three corresponding T-lymphocyte populations. These results are consistent with ZEBOV Sag activity, the first time that it is suggested in the Filoviridae family (Baize et al Journal of Virology April 2011 p 4041 4042 Letter to the Editor: Evidence for Ebola Virus Superantigen Activity). These findings may represent the missing link in our understanding of ZEBOV pathogenicity. The SAg activity of ZEBOV might contribute to the extraordinarily rapid and profound T-lymphocyte depletion observed during fatal infection. The high viral load observed during fatal ZEBOV infection, together with the simultaneous targeting of three Vβ T-cell subsets observed here, would elicit massive lymphocyte activation, rapidly resulting in the deletion of large populations of Vβ12-, Vβ13.2-, and Vβ17-bearing T cells (supra).

Widespread activation of T and B cells in Ebola patients with acute Ebola infection suggests both T and B cell superantigen activity (McElroy et al PNAS 2015 112 15 4719-4724). Polyclonal non-specific B-cell activation was observed in patients with acute Ebola infection, which suggests the presence of a B cell superantigen which is followed by widespread apoptosis (supra).

Marburg virus (MARV) and Ebola virus (EBOV) cause severe hemorrhagic fever in primates. Earlier studies demonstrated that antibodies to particular epitopes on the glycoprotein (GP) of EBOV enhanced virus infectivity in vitro. Using MARV strains Angola and Musoke, Nakayama et al demonstrated that the infectivity of MARV-Angola in K562 cells was enhanced notably in the presence of Angola GP antisera (ie, FcR-dependent ADE), whereas Musoke GP antisera did not significantly enhance the infectivity of MARV-Angola or MARV-Musoke. This difference between the two MARV strains was also supported by the observation that immunization with Angola GP induced significantly higher numbers of B-cell clones with infectivity-enhancing properties than did immunization with Musoke GP. These results may suggest that the potential difference in the pathogenicity between the MARV strains Angola and Musoke might be partially explained by the ability to induce infectivity-enhancing antibodies, as was proposed for the distinct pathogenicity seen with ZEBOV and Reston EBOV (Nakayama et al J Infect Dis. 2011 Nov. 1 204(Suppl 3) S978-S985). This observation suggests that the difference in the pathogenicity of ZEBOV and REBOV appears to be related to the B cell superantigen activity of ZEBOV verses REBOV and further suggests that ADE is of clinical importance in Ebola infections. Both B cell superantigen and pathogen immunoglobulin binding factors appear to play a role in the pathogenicity and that blocking the immunoglobulin binding factors appears to abrogate the increased pathogenicity associated with ADE.

The invention will be further described in the following examples, which do not limit the scope of the invention described in the claims.

### EXAMPLES

### Example 1

### Geographic and Temporal Co-Location of Ebola and Possible Cofactor Diseases

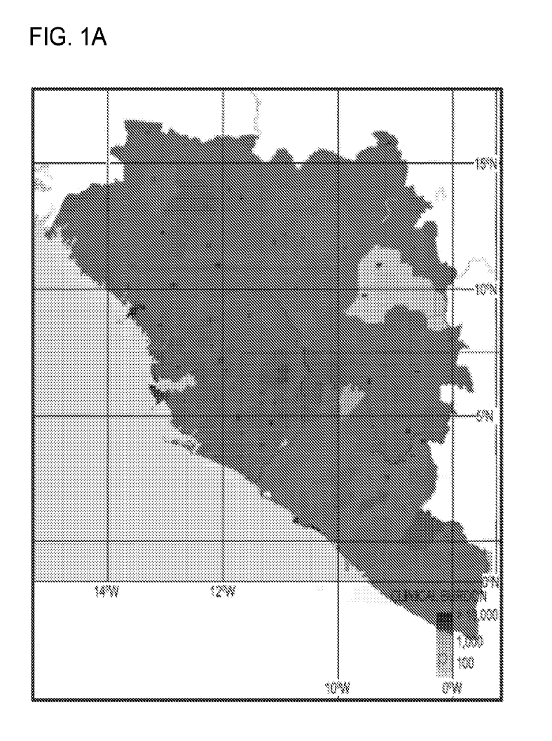

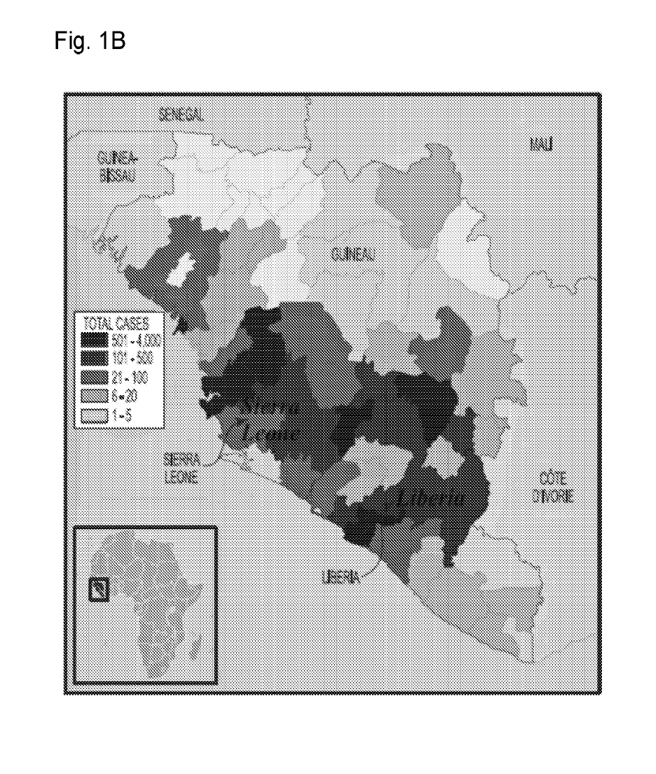

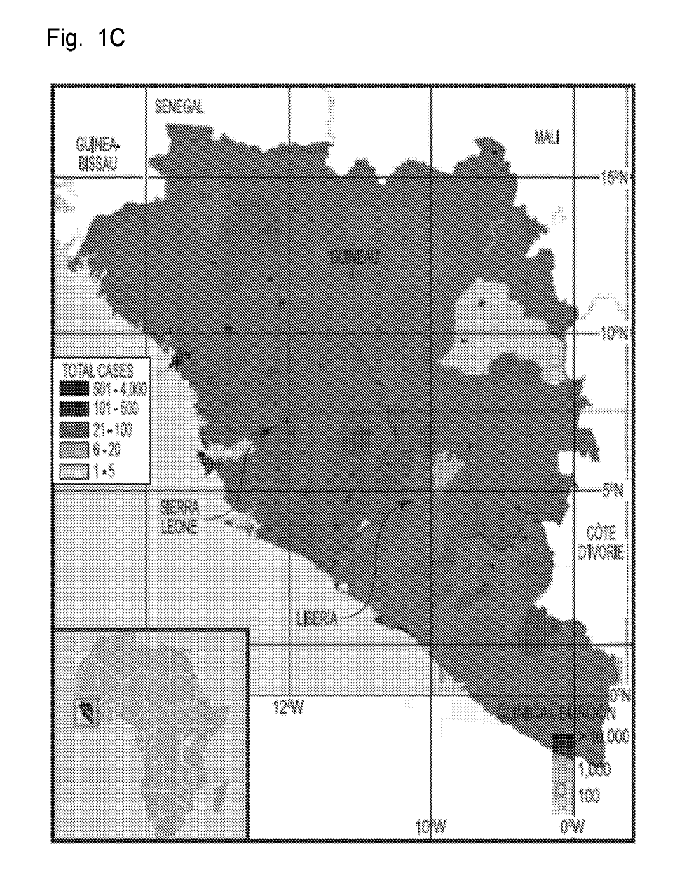

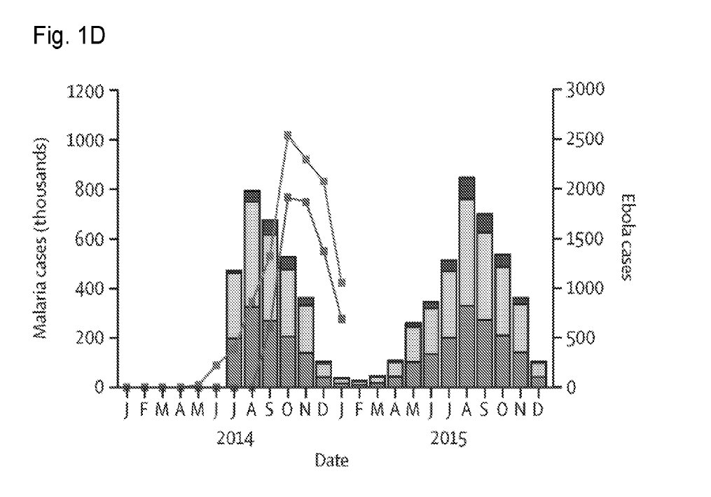

The clinical burden for serious Malaria in the countries of Liberia, Sierra Leone, and Guinea is shown in FIG. 1A, and a map of reported Ebola outbreaks as of the end of October 2014 is shown in FIG. 1B (MMWR CDC Nov. 18 2014 Ebola Virus Disease Epidemic—West Africa November 2014, available on the world wide web at cdc.gov/mmwr/pdf/wk/mm63e1118.pdf). A composite map of Ebola cases and serious Malaria cases, showing a geographic correlation, is shown in FIG. 1C. A clear correlation between simple uncomplicated Malaria and the present Ebola outbreak, on the other hand, is not evident (data not shown).

The observed geographic correlation of serious Malaria cases and the Ebola outbreak is consistent with the proposition that coinfection with other serious pathogenic agents may function as a cofactor for Ebola. West Africa, the site of the unprecedented 2014 Ebola outbreak, represents a region having one of the highest numbers of serious Malaria cases (Hay et al., PLOS Med., 2010, 7(6):e1000290). Also consistent with the coinfection cofactor proposition are the observations that some Ebola infections are asymptomatic (Becquart et al., PLOS One, 2010, 5(2):e9126), and that Ebola outbreaks have not occurred in other parts of the world, especially in the West where the incidence of co-infection other pathogenic agents is substantially lower.

The disruption of health services in the three Ebola outbreak affected countries has been shown to contribute to a higher number of malaria deaths in those countries (Walker et al., Lancet Infectious Dis., 2015, Epub Apr. 24, 2015 “Malaria morbidity and mortality in Ebola-affected countries caused by decreased health-care capacity, and the potential effect of mitigation strategies: a modelling analysis”). In addition, a seasonal or temporal relationship was found to exist between malaria deaths and Ebola cases in Guinea, Sierra Leone and Liberia. FIG. 1D shows malaria deaths as red bars, the green line is suspected or confirmed Ebola cases and the blue line is confirmed Ebola cases as reported by the WHO. As can be seen from FIG. 1D, the number of Ebola cases follows the number of malaria deaths in a seasonal or temporal fashion.

The seasonal/temporal relationship reinforces the geographic relationship between serious malaria/malaria deaths and Ebola cases.

### Example 2

### Coinfection of Ebola and PRRSV in Pigs

Reston Ebola virus (Reston ebolavirus; RESTV or REBOV), has been reported to cause disease in nonhuman primates and pigs, but not in humans. REBOV has been found to exist in domestic swine co-infected with porcine reproductive and respiratory syndrome virus (PRRSV). (Barrette et al., Science, 2009, 325(5937):204-206; Pan et al., Arch Virol., 2014, 159(5):1129-1132.) The observed incidence of PRRSV coinfection in REBOV-infected pig populations is likewise consistent with a proposed disease mechanism wherein REBOV replication is enhanced by coinfection, for example by immune complex (IC) formation induced by a non-related virus, PRRSV.

### Example 3

### Reverse ELISA Competitive Inhibition Bioassay for NB406 (SEQ ID NO:20) and Related Peptides

In vitro assays involving enzyme-linked immunosorbent assay (ELISA) can be used to demonstrate competitive inhibition of immune complexed IgG Fc binding to immune mediating factors such as Fc receptors (FcRs), (e.g., FcγRI, FcγIIa, FcγRIIb/c, FcγRIIIa/b), FcRn, mC1q, and sC1q by the polypeptides and compounds described herein. See, e.g., the examples described herein, as well as U.S. Patent Publication Nos. 20070225231 and 20070276125, and PCT Publication No. WO2007/030475. Standardized reagents and ELISA kits are useful to reduce costs and increase the reproducibility of the experiments.

Because SEQ ID NO:20 is a classic noncompetitive allosteric inhibitor designed to bind to the Fc region of IgG immune complexes (IC) and prevents IgG-IC from binding to Fc receptors (FcRs), we were able to develop an enzyme-linked immunosorbent assay (ELISA) to measure the bioactivity of SEQ ID NO:20. In a standard ELISA, an antigen is immunoadsorbed onto a plastic microwell. After suitable blocking and washing steps, a primary antibody with specificity directed toward the antigen is added to the microwell. After another wash phase, a secondary antibody that is directed toward the primary antibody and conjugated to an enzyme marker, such as horseradish peroxidase (HRP), is added to the microwell. Following another wash cycle, the appropriate enzyme substrate, such as 2,2′-azino-bis(3-ethylbenzothiazoline-6-sulphonic acid) (ABTS) in the case of HRP, is added. If an antigen to primary antibody to secondary antibody/HRP conjugate is formed, the conjugated enzyme catalyzes a colorimetric chemical reaction with the substrate, which is read with a microplate reader or spectrophotometer. By standardizing the levels of the antigen and secondary antibody/HRP conjugate, a titer of the primary antibody (the variable) is established. In a standard ELISA, the primary antibody binds to the antigen through its complementarily determining regions (CDR) located on the Fab arms. Because HRP is conjugated to the Fc region of the secondary antibody, direct Fc binding is very limited or abrogated.

A “reverse” ELISA technique is used to assess binding of the Fc receptor ligands that bind to IgG-IC. In the reverse ELISA, the enzyme (e.g., HRP) is not covalently conjugated to the Fc portion of the secondary antibody. Rather, a preformed immune complex of peroxidase-rabbit anti-peroxidase IgG (“PAP” complex) is used. SEQ ID NO:20 was designed to bind to human IgG-IC and since rabbit IgG is virtually identical to human IgG at the binding site for SEQ ID NO:20, rabbit IgG-IC can be used as a substitute for human IgG-IC, which are not commercially available. In this method, HRP serves both as the antigen and the enzyme marker but does not block the Fc region. In the reverse ELISA system, a Fc C_{H}2-C_{H}3 cleft binding ligand or FcR (e.g., human FcRIIa or C1q) is bound to microwell plates. In the absence of competitor, PAP IC complexes bind to the immobilized ligand and the reaction between HRP and its substrate produces a signal (“negative control”). This signal is reduced by the pre-incubation of inhibitors (e.g., FcRIIa, C1q or rheumatoid factor) or peptides such as SEQ ID NO:20 that inhibits PAP IC from binding to the immobilized FcR ligand. In a “reverse” inhibitive ELISA, less color development is indicative of greater inhibition. See, e.g., U.S. Pat. Nos. 6,916,904, 7,714,104, 8,362,202 and 8,815,813.

### A. Reverse ELISA Methodology.

All steps are performed at room temperature unless noted otherwise.

Preparing the FcR Coated Microwell Plates.

100 uL of a 0.05 ug/uL solution of human FcRIIA (rhFcRIIa) or C1q is used to coat each well that is to be used of a standard microwell plate that has 96 0.32 cm² wells. The rhFcRIIa is prepared by mixing 0.1 mL of 100 ug/200 uL rhFcRIIa, Santa Cruz Biotechnology, catalogue number sc-174810, or R&D Systems, catalogue number 1330-CD-050/CF, with 0.1 mL of 10× ELISA Plate Coating Buffer, AlphaDiagnostic International, catalogue number 80050, and 0.8 mL of 1×PBS. After allowing the microwell plate to incubate long enough to allow the rhFcRIIa to be immunoadsorbed onto the wells of the microwell plate (≧12 hours @ 4° C.), the rhFcRIIa solution is manually removed from the microwells. The plates are blocked from any further non-specific protein adsorption by adding 100 uL of 1 mg/mL bovine serum albumin (BSA), Cohn fraction V, Sigma-Aldrich, catalogue number A5611, to each of the wells. The 1 mg/mL BSA solution is prepared by dissolving the BSA in 1×PBS and then filter sterilizing. After allowing the plate to incubate for 1 hour @ 4° C., the BSA solution is manually removed and each well is washed four more times with 100 uL of the 1 mg/mL BSA solution with no additional time between washes. One final wash with 100 uL of 1×PBS is then done to remove any residual BSA. If the plate is sealed it can be used for up to several days later.

Forming the PAP Immune Complex (IC).

10 mg of HRP, Sigma-Aldrich, catalogue number P6782, is dissolved in 2 mL of 1×PBS. Lyophilized rabbit anti-peroxidase antibody, Sigma-Aldrich, catalogue number P7899, is dissolved in 2.1 mL of ice cold 1×PBS and filtered using a 0.22 μm pore size 13 mm PVDF syringe filter and a 3 mL sterile syringe. The resuspended rabbit anti-peroxidase antibody is stored at 4° C. The rabbit antibody must be handed using sterile techniques to avoid bacterial contamination since the Sigma P7899 antibody does not contain a preservative agent. The resuspended antibody can be used up to one year as long as no contamination occurs as judged by an increase in turbidity. To form the PAP complex, 20 uL of the resuspended rabbit anti-peroxidase antibody is added to 950 μl of 1×PBS, followed by the addition of 50 μl of the 5 mg/mL HRP solution. The PAP complex must be used immediately. It cannot be stored, because even with antigen excess, larger immune complexes will be formed upon storage. The extreme antigen excess guarantees that any single antibody is bound to two HRP molecules and prevents larger immune complexes from forming, thus eliminating the bridging of larger immune complexes. Potential inhibitors are dissolved in 1×PBS and 100 uL of the inhibitor to be tested is then added to 100 uL of the PAP complex and allowed to incubate for 1 hour. For a control, 100 uL of 1×PBS is added to 100 uL of the PAP complex instead of the inhibitor.

Performing the Reaction.

100 uL of each inhibitor/PAP complex mixture is added to a well of the coated microwell plate as well as the control PAP complex without the added inhibitor. After allowing the plate to incubate for 1 hour, the inhibitor/PAP complex mixture is manually removed and each well is washed four more times with 100 uL of 1×PBS with no additional time between washes. 100 uL of SUB4 ABTS 1-Component HRP Microwell Substrate, ImmunoChemistry Technologies, catalogue number 6278, is added to each well and after 30 minutes of incubation, 100 uL of Stop Solution for ABTS Microwell Substrate STOP2, ImmunoChemistry Technologies, catalogue number 6283, is added to each well and the color development is read at OD 405 nm on a microwell ELISA reader or spectrophotometer.

B. Inhibition of C1q Binding to PAP Immune Complex (see U.S. Pat. No. 8,815,813)

Exemplary materials and methods are described in more detail above. Briefly, C1q was immobilized on the microwell plates. PAP immune complexes were formed by mixing 2 μl of rabbit anti-peroxidase (Sigma Chemicals Product P 7899) with 50 μl of peroxidase (Sigma-Aldrich P6782) in 1 ml distilled water. PAP (100 μl) were pre-incubated with 100 μl of human C1q (Quidel Corp.) or 100 μl of peptide (see Table 1) for one hour. Like FcR, C1q is known to bind to the Fc region on IgG. The ability of PAP immune complexes to bind to the immobilized C1q in the presence or absence of inhibitors was determined. The C1q/PAP mixture served as a positive control; PAP immune complexes bind to soluble C1q, and the premixed C1q/PAP immune complexes are therefore not expected to bind to the C1q-containing plate, resulting in low signal levels, similar to what would be observed in the presence of an inhibitor. The PAP immune complex alone serves as the negative control; in the absence of inhibitors, the PAP immune complex is expected to bind to the C1q-containing plate, resulting in high signal levels.

C1q/PAP mixture, peptide/PAP mixtures, and PAP alone were incubated with C1q coated plates for 30 minutes. After washing, plates were incubated with ATBS (Quidel Corp.) for 15 minutes and read at 405 nm. Results are shown in Table 1.

TABLE 1

| | SEQ | |
|---|---|---|
| Peptide | ID NO: | OD 405 nm |
| DCAWHLGELVWCT | 2 | 1.100 |
| APPCARHLGELVWCT | 16 | 0.567 |
| DCAFHLGELVWCT | 3 | 0.859 |
| APPDCAWHLGELVWCT | 20 | 0.389 |
| APPCAFHLGELVWCT | 18 | 0.983 |
| APPCAWHLGELVWCT | 14 | 1.148 |
| C1q (positive control) | — | 0.337 |
| Negative Control (no inhibitor) | — | 2.355 |

APPDCAWHLGELVWCT (SEQ ID NO:20) resulted in the greatest inhibition of C1q binding, almost equaling C1q itself. Peptide APPCARHLGELVWCT (SEQ ID NO:16) gave the next best result.

### C. Inhibition of FcR Binding to PAP Immune Complex

Once the reverse ELISA protocol was established using the C1q assay, the assay was redesigned using FcγIIa, FcγIIb and FcγIII in place of C1q. Highly purified FcγIIa, FcγIIb and FcγIII (R&D Systems, Minneapolis, Minn.) were immunoadsorbed onto plastic microwells. After optimizing the FcγR reverse ELISA system, simple competitive inhibition experiments using polypeptides provided herein were conducted to investigate their ability to inhibit binding of immune complexes to purified FcγR.

Exemplary materials and methods are described in more detail above. Briefly, Falcon microtiter plates were coated with 1:10 dilutions of highly purified FcγIIa, FcγIIb and FcγIII and incubated for 24 hours. The plates were washed and then blocked with 5.times.BSA blocking solution (Alpha Diagnostic International, San Antonio, Tex.) for 24 hours. PAP immune complexes were formed by mixing 2 μl of rabbit anti-peroxidase (Sigma Chemicals Product P 7899) with 50 μl of peroxidase (Sigma-Aldrich P6782) in 1 ml distilled water. PAP (100 μl) were pre-incubated with 100 l of peptide for one hour. PAP/peptide mixtures were added to the FcγR coated plates and incubated for one hour. After washing, plates were incubated with ABTS substrate (Quidel Corp.) for 15 minutes and read at 405 nm. Results are shown in Table 2.

TABLE 2

| | | OD₄₀₅ | | |
|---|---|---|---|---|
| | | FcγIIa | | |
| Peptide | SEQ ID | (R131 allele) | FcγIIb | FcγIIIa |
| DCAWHLGELVWCT | 2 | 0.561 | 0.532 | 0.741 |
| APPCARHLGELVWCT | 16 | 0.956 | 0.768 | 0.709 |
| DCAFHLGELVWCT | 3 | 0.660 | 0.510 | 0.810 |
| APPDCAWHLGELVWCT | 20 | 0.509 | 0.496 | 0.670 |
| APPCAFHLGELVWCT | 18 | 0.605 | 0.380 | 0.880 |
| APPCAWHLGELVWCT | 14 | 0.658 | 0.562 | 0.530 |
| Negative Control | — | 1.599 | 1.394 | 1.588 |
| (no inhibitor) | | | | |

Peptide APPDCAWHLGELVWCT (SEQ ID NO:20) appeared to result in the greatest overall inhibition of FcR binding to PAP, followed by peptide DCAWHLGELVWCT (SEQ ID NO:2). Experiments with SEQ ID NO:20 were repeated. Costar microtiter plates were coated with 1:10 dilutions of highly purified FcγIIa (H is 131 allele aka H161), FcγIIb and FcγIIIb and incubated for 24 hours. The plates were washed and then blocked with 10 mg/ml BSA blocking solution for 24 hours. PAP immune complexes were formed as described in Example 2. PAP (100 μl) were pre-incubated with 100 μl of peptide for one hour. PAP/peptide mixtures were added to the FcγR coated plates and incubated for one hour. After washing, plates were incubated with ABTS substrate for 15 minutes and read at 405 nm. Results are shown in Table 3.

TABLE 3

| | SEQ | | | | FcγIIIb |
|---|---|---|---|---|---|
| Peptide | ID | FcγI | FcγIIa* | FcγIIb/c | (NA2) |
| APPDCAWHLGELVWCT | 20 | 0.264 | 0.209 | 0.266 | 0.266 |
| Negative control (no | — | 2.333 | 3.509 | 2.218 | 3.060 |
| inhibitor) | | | | | |
| % inhibition of IC binding | — | 89 | 94 | 88 | 91 |

*Note:
(H131 allele)

Thus, SEQ ID NO:20 inhibited binding of all three major classes of Fc receptor (FcγI, FcγIIa/FcγIIb, and FcγIII) to soluble PAP immune complexes.

### D. Ebolavirus as a Viral FcR

Demonstration that Ebolavirus Contains a Viral FcR and that NB406 (SEQ ID NO:20) can Prevent the Binding of IC to Ebolavirus

To test whether the Ebolavirus contained an FcR and whether SEQ ID NO:20 could inhibit the binding of IC to the FcR of the Ebolavirus, we used the reverse ELISA assay. Instead of binding a known FcR to the plate, such as human FcRIIa, or C1q, Sudan Ebolavirus, Boniface, was used, which was obtained from Bei Resources, Number NR-31810. 100 uL of a 1:100 dilution from 100 uL of sonicated Vero E6 cell pellets that had been infected with Sudan Ebolavirus, Boniface, frozen and then gamma-irradiated were used to coat each well of the microplate. The standard reverse ELISA protocol was implemented with the inhibitor SEQ ID NO:20 used at a concentration of 10 mg/mL, 3 mg/mL, and 1 mg/mL; OD was read at 405 nm.

NB406 (SEQ ID NO: 20) Inhibits the Binding
of IC to Sudan Ebolavirus (SEBOV)

| IC +/− SEQ | 1:100 | Percent | 1:1000 | Percent | 1:10000 | Percent |
|---|---|---|---|---|---|---|
| ID NO: 20 | SEV | Inhibition | SEV | Inhibition | SEV | Inhibition |
| 0 | 1.415 | | 1.288 | | 1.084 | |
| 10 mg/mL | 0.048 | 96.6% | 0.048 | 96.3 | 0.040 | 96.3% |
| 3 mg/mL | 0.069 | 95.1% | 0.069 | 94.6% | 0.058 | 94.6% |
| 1 mg/mL | 0.317 | 77.6% | 0.453 | 64.8% | 0.449 | 58.6% |
| HRP only | 0.251 | | 0.168 | | 0.038 | |

Sudan Ebolavirus (SEBOV) was tested at several concentrations and either 1:100, 1:1,000 or 1:10,000 dilutions of the stock virus was used in the standard reverse ELISA.

The results demonstrate that Ebolavirus contains a viral FcR that can bind IC and that SEQ ID NO:20 can effectively prevent the binding of IC to the Ebola virus.

Demonstration that Ebolavirus Glycoprotein is a Viral FcR and that NB406 (SEQ ID NO:20) can Prevent the Binding of IC to Ebolavirus Glycoprotein

Once it was established that whole Ebolavirus can bind an IC and thus acts as a viral Fc receptor (FcR), it was of interest to determine which protein constituent of Ebolavirus contains the viral FcR binding activity. Ebolavirus glycoprotein (GP) was investigated as a candidate viral FcR. The reverse ELISA assay was used to test whether the Ebolavirus glycoprotein was the viral FcR and whether SEQ ID NO:20 could inhibit the binding of IC to the glycoprotein viral FcR of the Ebolavirus. Instead of binding a known FcR to the plate, such as human FcRIIa, C1q, or whole Ebolavirus, as in the preceding section, recombinant Sudan Ebolavirus glycoprotein from IBT Bioservices, Catologue Number 0501-015, was used. 100 uL of a 10 ug/mL solution of Sudan Ebolavirus glycoprotein was used to coat each well of the microplate. The standard reverse ELISA protocol was implemented with the inhibitor SEQ ID NO:20 used at a concentration of 10 mg/mL, 3 mg/mL, and 1 mg/mL; OD was read at 405 nm.

NB406 (SEQ ID NO: 20) Inhibits the Binding
of IC to Sudan Ebolavirus Glycoprotein (GP)

| IC +/− SEQ | 10 ug/mL | Percent | 3 ug/mL | Percent | 1 ug/mL | Percent |
|---|---|---|---|---|---|---|
| ID NO: 20 | GP | Inhibition | GP | Inhibition | GP | Inhibition |
| 0 | 1.613 | | 1.375 | | 1.314 | |
| 10 mg/mL | 0.046 | 97.1% | 0.042 | 96.9% | 0.042 | 96.8% |
| 3 mg/mL | 0.058 | 96.4% | 0.046 | 96.7% | 0.053 | 96.0% |
| 1 mg/mL | 0.095 | 94.1% | 0.081 | 94.1% | 0.071 | 94.6% |
| HRP only | 0.144 | | 0.172 | | 0.139 | |

Sudan Ebolavirus Glycoprotein (GP) was tested at several concentrations and either 10 ug/mL, 3 ug/mL or 1 ug/mL concentrations were used in the standard reverse ELISA.

The results demonstrate that Ebolavirus glycoprotein (GP) is a viral FcR that can bind IC and that SEQ ID NO:20 can effectively prevent the binding of IC to the Ebolavirus glycoprotein.

### Example 4

### Inhibition of Recombinant Human FcγRIIa Binding to Ebolavirus Immune Complex

In this experiment, Ebolavirus (Sudan) immune complexes are evaluated for binding to immobilized recombinant human FcγRIIa.

Highly purified recombinant human FcγRIIa (R&D Systems, 1 μg/ml) are immunoadsorbed onto plastic microwells (Costar Microtiter plates) using ELISA coating buffer (Alpha Diagnostic International) for 24 hours at 4° C. After 24 hours, the plates were washed 5 times with ELISA wash solution (Quidel Corp.), blocked with highly purified BSA for 1 hour, and washed 5 times. Rabbit anti-SEBOV IC are formed by combining whole inactivated Sudan Ebola virus (SEBOV-Sudan Ebolavirus, Boniface, Gamma-Irradiated—SEBOV BEI RESOURCES) with IBT Bioservices 0302-020 Rabbit Polyclonal anti-Sudan GP. Rabbit anti-SEBOV GP IC (100 μl) is pre-incubated with and without of SEQ ID NO:20 (100 μl) for one hour. Next, either rabbit-anti-SEBOV IgG IC (100 μl) or rabbit-anti-SEBOV IgG-NB406 (100 μl) is added to the FcγRIIa coated plates. After one half hour incubation, the plates are washed five times. After one hour, the plates are washed and 100 μl of 1:1,000 dilution of Pierce Mouse anti-Rabbit IgG (H+L) Cross Adsorbed Secondary Antibody, HRP conjugate Cat #31464 is added to the microwells and incubated for one hour. 100 μl of ABTS are added to each micro-well, after ½ hour incubation, the plates are read at 405 nm.

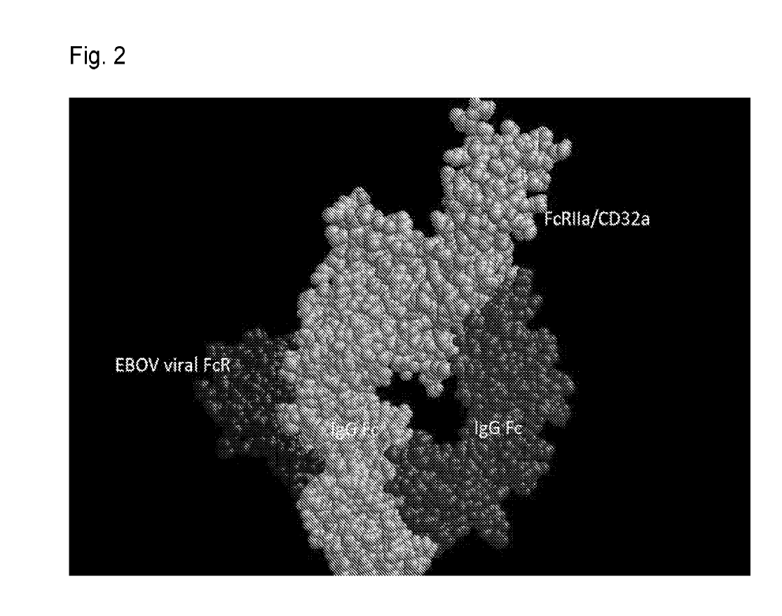

As shown in FIG. 2, immune complexes, in vitro and in vivo, may bridge Ebola viral FcR to cellular FcγR, primarily recombinant human FcγRIIa, and allow infection (either cis or trans) of immunocompetent cells, such as macrophages. As shown in FIG. 2, FcRIIa/CD32a (GOLD) can bind to to IgG Fc (RED & GREEN), and the IgG Fc is also bound to EBOV viral FcR (BLUE). In addition to the classic IC-FcR interaction, the EBOV viral FcR allows an alternate way to get ADE. SEQ ID NO:20 (NB406) disrupts IgG-Fc to Fc interaction and inhibits both the FcRIIa/CD32a to IgG Fc binding and the EBOV viral FcR to IgG Fc binding.

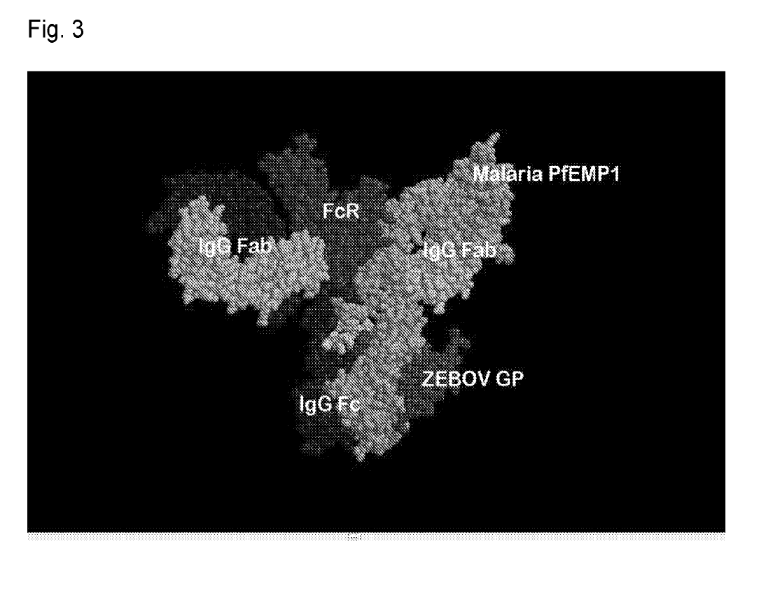

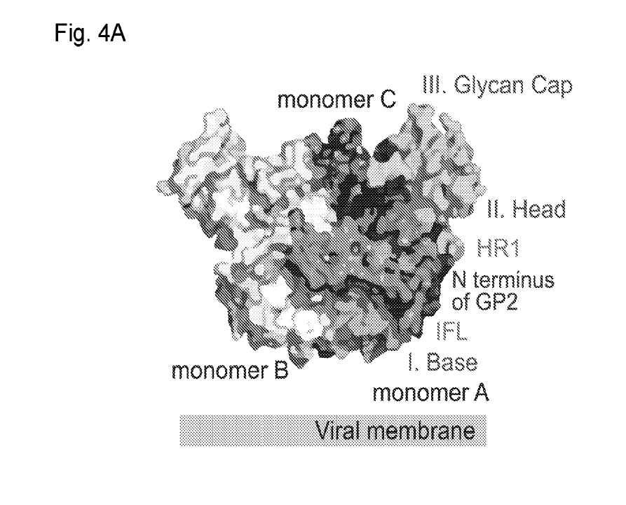

As shown in FIG. 3, an immune complex that includes an antibody that recognizes a non-Ebola antigen including, for example, a Malaria antigen (PfEMP1, GOLD), can also bridge Ebola to a human cell. As shown in FIG. 3, the IgG (DARK BLUE, LIGHT BLUE) binds to PfEMP1 (GOLD) though the Fab portion of the antibody, the Fc portion binds to EBOV viral FcR (PURPLE), and the hinge region binds to human FcR (RED).

### Example 5

### Preventing Antibody Dependent Enhancement of Ebolavirus Infection

U937 or K562 Monocyte cell types expressing both FcγRIIa and FcγRIIb are infected with vesicular stomatitis virus pseudotyped with EBOV GP in the manner described by Takada et al 2007 (J Infect Dis 2007 196 S2 S347). Diluted convalescent Ebola sera are added to produce antibody dependent enhancement (ADE) of infection of the U937 or K562 cells.

Monoclonal antibodies to FcγRIIb (SuppreMol SM201 or Xencor XmAb5871) are added to the VSV-EBOV GP/immune complex/cell cultures, and ligation of FcγRIIb is expected to result in an inhibition of ADE in cell culture.

### Example 6

### Contrasting Filovirus Infection and Flavivirus Infection

A major difference in antibody dependent enhancement (ADE) of filoviruses (Ebola and Marburg) and flaviviruses (dengue and West Nile Virus (WNV)) is that both C1q and FcγR cause or exacerbate ADE in filoviruses (Takada et al., J. Infect. Dis. 2007, 196(S2):S347-56; Nakayama et al., J. Infect. Dis., 2011, 204(S3):S978-85), whereas C1q decreases or inhibits ADE in dengue and WNV infections (Mehlhop et al., Cell Host Microbe, 2007, 2(6):417-26). Complement protein C1q inhibits antibody-dependent enhancement of flavivirus infection in an IgG subclass-specific manner (Mehlhop et al., Cell HostMicrobe, 2009, 6(4):381-91). There are both stoichiometric and conformational reasons for this difference. For example, Ebola GP consists of three GP1/GP2 molecules forming a trimer. The trimer forms a “chalice” (FIG. 4A):

C1q binds IgG hexamers (Diebolder et al., Science, 2014, 343(6176): 1260-3). The IgG hexamers form a ring structure that allows high affinity binding by C1q (FIG. 4B).

The hexametric C1q binds to these IgG hexamers. The mechanism of action for SEQ ID NO:20 (NB406) inhibition of C1q to IC binding does not appear to be due to allosteric inhibition, but rather disruption of the IgG Fc-Fc interactions required to form the IgG hexamer. In examining the structure of Ebola GP, we surmised that two Ebola GP trimers could be mimicking C1q and the mechanism of action for inhibiting Ebola GP binding to IgG IC may be due to disruption of Fc-Fc interactions. FIG. 4C shows the results of in silco modeling that shows that it is possible that Ebola GP is binding two or three IgGs present in the IgG hexamer.

In this model, SEQ ID NO:20 (NB406) inhibits Ebola GP to IgG (hexamer) binding by disrupting the Fc-Fc interactions required to form the IgG hexamer. The idea that Ebola GP is mimicking C1q is consistent with the experimental data disclosed herein, but is completely unexpected.

### Example 7

### SEQ ID NO:20 can be Used as a Treatment for Influenza Virus Infections

This Example describes evaluation of whether the influenza virus could bind to an immune complex (IC) and whether NB406 (SEQ ID NO:20) could prevent this binding from occurring. While most researchers argue that the greater susceptibility of patients with autoimmune diseases to influenza infections is due to their compromised health, we believe that the influenza virus uses immune complexes as a mechanism to hijack the immune system and proliferate more rapidly. For this reason we wanted to determine whether the influenza virus contained Fc receptors (FcRs) that would bind to ICs and whether the NB406 peptide (SEQ ID NO:20) could prevent this binding from occurring and thus act a potential therapeutic agent to prevent the proliferation of influenza infections.

A. Detecting and Quantifying the Binding of ICs to FcRs and Inhibitors that can Prevent this Binding from Occurring.

ICs are key to the immune response and occur when an IgG binds to an antigen. ICs are prevalent in any disease or condition that stimulates an immune response, including autoimmune diseases. The ICs that are formed then bind to FcRs or C1q, which causes the immune response to progress. A multitude of approaches and methods have been developed to detect and quantify ICs, since they are a hallmark of a variety of diseases. We have developed a simple but robust reverse ELISA method for quantifying the ability of potential inhibitor compounds to prevent the binding of ICs to FcRs and to determine whether an entity, such as a virus or cell, contains any FcRs. In this method horseradish peroxidase (HRP) and anti-HRP IgG antibody is mixed to form an IC, which is then bound to an FcR, such as human FcRIIa or C1q, or a cell or virus that is suspected to contain a FcR. The reverse ELISA was originally described in U.S. Pat. No. 6,916,904 and implemented in U.S. Pat. No. 6,916,904. The experiments described here were performed using the protocols described in U.S. Pat. Nos. 6,916,904 and 7,714,104, with an expanded explanation of the principles and protocols used in the bioassay.

In a standard ELISA, an antigen is immunoadsorbed onto a plastic microwell. After suitable blocking and washing steps, a primary antibody with specificity directed toward the antigen is added to the microwell. After another wash phase, a secondary antibody that is directed toward the primary antibody and conjugated to an enzyme marker, such as horseradish peroxidase (HRP), is added to the microwell. Following another wash cycle, the appropriate enzyme substrate, such as 2,2′-azino-bis(3-ethylbenzothiazoline-6-sulphonic acid) (ABTS) in the case of HRP, is added. If an antigen to primary antibody to secondary antibody/HRP conjugate is formed, the conjugated enzyme catalyzes a colorimetric chemical reaction with the substrate, which is read with a microplate reader or spectrophotometer. By standardizing the levels of the antigen and secondary antibody/HRP conjugate, a titer of the primary antibody (the variable) is established. In a standard ELISA, the primary antibody binds to the antigen through its complementarily determining regions (CDR) located on the Fab arms. Because HRP is conjugated to the Fc region of the secondary antibody, direct Fc binding is very limited or abrogated.

In our reverse ELISA technique, the enzyme (e.g., HRP) is not covalently conjugated to the Fc portion of the secondary antibody. Rather, a preformed immune complex of peroxidase-rabbit anti-peroxidase IgG (“PAP” complex) is used. In this method, HRP serves both as the antigen and the enzyme marker but does not block the Fc region. In the reverse ELISA system, a Fc C_{H}2-C_{H}3 cleft binding ligand or FcR (e.g., human FcRIIa or C1q) is bound to microwell plates. In the absence of a competitor, PAP IC complexes bind to the immobilized ligand and the reaction between HRP and its substrate produces a signal (“Positive Control”). This signal is reduced by the pre-incubation of inhibitors (e.g., FcRIIa, C1q, rheumatoid factor or peptides, such as SEQ ID NO:20) that prevent PAP IC from binding to the immobilized FcR ligand. In our reverse ELISA, less color development is indicative of greater inhibition.

### B. NB406 (SEQ ID NO:20)

NB406 (SEQ ID NO:20) is a classic noncompetitive allosteric inhibitor designed to bind to the Fc region of human IgG IC and prevent IgG-IC from binding to FcRs (U.S. Pat. No. 6,916,904 and U.S. Pat. No. 6,916,904). Because NB406 (SEQ ID NO:20) was designed to bind to human IgG-IC and since rabbit IgG is virtually identical to human IgG at the NB406 binding site, rabbit IgG-IC can be used as a substitute for human IgG-IC, which are not commercially available, in either in vitro or in vivo testing.

### Protocol (all Steps are Performed at Room Temperature Unless Noted Otherwise)

### Preparing the FcR Coated Microwell Plates

100 uL of a 0.05 ug/uL solution of human FcRIIA (rhFcRIIa) or C1q is used to coat each well that is to be used of a standard microwell plate that has 96 0.32 cm² wells. The rhFcRIIa is prepared by mixing 0.1 mL of 100 ug/200 uL rhFcRIIa, Santa Cruz Biotechnology, catalogue number sc-174810, with 0.1 mL of 10× ELISA Plate Coating Buffer, AlphaDiagnostic International, catalogue number 80050, and 0.8 mL of 1×PBS. After allowing the microwell plate to incubate long enough to allow the rhFcRIIa to be immunoadsorbed onto the wells of the microwell plate (>12 hours @ 4° C.), the rhFcRIIa solution is manually removed from the microwells. The plates are blocked from any further non-specific protein adsorption by adding 100 uL of 1 mg/mL bovine serum albumin (BSA), Cohn fraction V, Sigma-Aldrich, catalogue number A5611, to each of the wells. The 1 mg/mL BSA solution is prepared by dissolving the BSA in 1×PBS and then filter sterilizing. After allowing the plate to incubate for 1 hour @ 4° C., the BSA solution is manually removed and each well is washed four more times with 100 uL of the 1 mg/mL BSA solution with no additional time between washes. One final wash with 100 uL of 1×PBS is then done to remove any residual BSA. If the plate is sealed it can be used for up to several days later.

### Forming the PAP Complex

10 mg of HRP, Sigma-Aldrich, catalogue number P6782, is dissolved in 2 mL of 1×PBS. Lyophilized rabbit anti-peroxidase antibody, Sigma-Aldrich, catalogue number P7899, is dissolved in 2.1 mL of ice cold 1×PBS and filtered using a 0.22 μm pore size 13 mm PVDF syringe filter and a 3 mL sterile syringe. The resuspended rabbit anti-peroxidase antibody is stored at 4° C. The rabbit antibody must be handed using sterile techniques to avoid bacterial contamination since the Sigma P7899 antibody does not contain a preservative agent. The resuspended antibody can be used up to one year as long as no contamination occurs as judged by an increase in turbidity. To form the PAP complex, 20 uL of the resuspended rabbit anti-peroxidase antibody is added to 950 μl of 1×PBS, followed by the addition of 50 μl of the 5 mg/mL HRP solution. The PAP complex must be used immediately. It cannot be stored, because even with antigen excess, larger immune complexes will be formed upon storage. The extreme antigen excess guarantees that any single antibody is bound to two HRP molecules and prevents larger immune complexes from forming, thus eliminating the bridging of larger immune complexes. Potential inhibitors are dissolved in 1×PBS and 100 uL of the inhibitor to be tested is then added to 100 uL of the PAP complex and allowed to incubate for 1 hour. For a control, 100 uL of 1×PBS is added to 100 uL of the PAP complex instead of the inhibitor.

### Performing the Reaction

100 uL of each inhibitor/PAP complex mixture is added to a well of the coated microwell plate as well as the control PAP complex without the added inhibitor. After allowing the plate to incubate for 1 hour, the inhibitor/PAP complex mixture is manually removed and each well is washed four more times with 100 uL of 1×PBS with no additional time between washes. 100 uL of SUB4 ABTS 1-Component HRP Microwell Substrate, ImmunoChemistry Technologies, catalogue number 6278, is added to each well and after 30 minutes of incubation, 100 uL of Stop Solution for ABTS Microwell Substrate STOP2, ImmunoChemistry Technologies, catalogue number 6283, is added to each well and the color development is read at OD 405 nm on a microwell ELISA reader or spectrophotometer.

C. Demonstration that Influenza Virus Contains a FcR and that NB406 (SEQ ID NO:20) can Prevent the Binding of IC to Influenza Virus

To test whether the influenza virus contained a FcR and whether NB406 (SEQ ID NO:20) could inhibit the binding of IC to the FcR of influenza virus, we used the reverse ELISA assay. Instead of binding a known FcR to the plate, such as human FcRIIa or C1q, influenza virus, was used. 100 uL of a 1:10 or 1:100 dilution of Influenza A/MAL/NY/6750/78 H2N2 virus at a titer of 1.2×10⁶ used to coat each well of the microplate. The standard reverse ELISA protocol was implemented with the inhibitor NB406 used at a concentration of 10, 3.33 or 1.11 mg/mL. The assay was repeated three times and a typical result is shown in Table 4. Influenza A virus is abbreviated as IAV in Table 4.

TABLE 4

| IC +/− | 1:10 | Percent | 1:100 | Percent |
|---|---|---|---|---|
| NB406 | IAV | Inhibition | IAV | Inhibition |
| 0 | 1.936 | | 1.873 | |
| 10 mg/mL | 0.046 | 97.6% | 0.057 | 97.0% |
| 3.33 mg/mL | 0.066 | 96.6% | 0.074 | 96.0% |
| 1.11 mg/mL | 0.204 | 89.5% | 0.212 | 88.7% |
| 0.37 mg/mL | 0.707 | 63.5% | 0.719 | 61.6% |
| HRP only | 0.163 | | 0.178 | |

The results demonstrate that influenza virus contains a FcR that can bind IC and that NB406 can effectively prevent the binding of IC to the influenza virus.

### Example 8

### SEQ ID NO:20 can be Used as a Treatment for Hepatitis Virus Infections

This Example describes evaluation of whether the hepatitis virus could bind to an immune complex (IC) and whether NB406 (SEQ ID NO:20) could prevent this binding from occurring. While most researchers argue that the greater susceptibility of patients with autoimmune diseases to hepatitis viral infections is due to their compromised health, we believe that the hepatitis virus uses immune complexes as a mechanism to bridge the immune system and proliferate more rapidly. For this reason we wanted to determine whether the hepatitis A and C viruses contained Fc receptors (FcRs) that would bind to ICs and whether the NB406 peptide could prevent this binding from occurring and thus act a potential therapeutic agent to prevent the proliferation of hepatitis infections.

A. Detecting and Quantifying the Binding of ICs to FcRs and Inhibitors that can Prevent this binding from occurring.

ICs are key to the immune response and occur when an IgG binds to an antigen. ICs are prevalent in any disease or condition that stimulates an immune response, including autoimmune diseases. The ICs that are formed then bind to FcRs or C1q, which causes the immune response to progress. A multitude of approaches and methods have been developed to detect and quantify ICs, since they are a hallmark of a variety of diseases. We have developed a simple but robust reverse ELISA method for quantifying the ability of potential inhibitor compounds to prevent the binding of ICs to FcRs and to determine whether an entity, such as a virus or cell, contains any FcRs. In this method horseradish peroxidase (HRP) and anti-HRP IgG antibody is mixed to form an IC, which is then bound to an FcR, such as human FcRIIa or C1q, or a cell or virus that is suspected to contain a FcR. The reverse ELISA was originally described in U.S. Pat. No. 6,916,904 and implemented in U.S. Pat. No. 6,916,904. The experiments described here were performed using the protocols described in U.S. Pat. Nos. 6,916,904 and 7,714,104, with an expanded explanation of the principles and protocols used in the bioassay.

In a standard ELISA, an antigen is immunoadsorbed onto a plastic microwell. After suitable blocking and washing steps, a primary antibody with specificity directed toward the antigen is added to the microwell. After another wash phase, a secondary antibody that is directed toward the primary antibody and conjugated to an enzyme marker, such as horseradish peroxidase (HRP), is added to the microwell. Following another wash cycle, the appropriate enzyme substrate, such as 2,2′-azino-bis(3-ethylbenzothiazoline-6-sulphonic acid) (ABTS) in the case of HRP, is added. If an antigen to primary antibody to secondary antibody/HRP conjugate is formed, the conjugated enzyme catalyzes a colorimetric chemical reaction with the substrate, which is read with a microplate reader or spectrophotometer. By standardizing the levels of the antigen and secondary antibody/HRP conjugate, a titer of the primary antibody (the variable) is established. In a standard ELISA, the primary antibody binds to the antigen through its complementarily determining regions (CDR) located on the Fab arms. Because HRP is conjugated to the Fc region of the secondary antibody, direct Fc binding is very limited or abrogated.

In our reverse ELISA technique, the enzyme (e.g., HRP) is not covalently conjugated to the Fc portion of the secondary antibody. Rather, a preformed immune complex of peroxidase-rabbit anti-peroxidase IgG (“PAP” complex) is used. In this method, HRP serves both as the antigen and the enzyme marker but does not block the Fc region. In the reverse ELISA system, a Fc C_{H}2-C_{H}3 cleft binding ligand or FcR (e.g., human FcRIIa or C1q) is bound to microwell plates. In the absence of a competitor, PAP IC complexes bind to the immobilized ligand and the reaction between HRP and its substrate produces a signal (“Positive Control”). This signal is reduced by the pre-incubation of inhibitors (e.g., FcRIIa, C1q, rheumatoid factor or peptides, such as SEQ ID NO:20) that prevent PAP IC from binding to the immobilized FcR ligand. In our reverse ELISA, less color development is indicative of greater inhibition.

### B. NB406 (SEQ ID NO:20)

NB406 (SEQ ID NO:20) is a classic noncompetitive allosteric inhibitor designed to bind to the Fc region of human IgG IC and prevent IgG-IC from binding to FcRs (U.S. Pat. No. 6,916,904 and U.S. Pat. No. 6,916,904). Because NB406 (SEQ ID NO:20) was designed to bind to human IgG-IC and since rabbit IgG is virtually identical to human IgG at the NB406 binding site, rabbit IgG-IC can be used as a substitute for human IgG-IC, which are not commercially available, in either in vitro or in vivo testing.

### Protocol (all Steps are Performed at Room Temperature Unless Noted Otherwise)

### Preparing the FcR Coated Microwell Plates

100 uL of a 0.05 ug/uL solution of human FcRIIA (rhFcRIIa) or C q is used to coat each well that is to be used of a standard microwell plate that has 96 0.32 cm² wells. The rhFcRIIa is prepared by mixing 0.1 mL of 100 ug/200 uL rhFcRIIa, Santa Cruz Biotechnology, catalogue number sc-174810, with 0.1 mL of 10× ELISA Plate Coating Buffer, AlphaDiagnostic International, catalogue number 80050, and 0.8 mL of 1×PBS. After allowing the microwell plate to incubate long enough to allow the rhFcRIIa to be immunoadsorbed onto the wells of the microwell plate (>12 hours @ 4° C.), the rhFcRIIa solution is manually removed from the microwells. The plates are blocked from any further non-specific protein adsorption by adding 100 uL of 1 mg/mL bovine serum albumin (BSA), Cohn fraction V, Sigma-Aldrich, catalogue number A5611, to each of the wells. The 1 mg/mL BSA solution is prepared by dissolving the BSA in 1×PBS and then filter sterilizing. After allowing the plate to incubate for 1 hour @ 4° C., the BSA solution is manually removed and each well is washed four more times with 100 uL of the 1 mg/mL BSA solution with no additional time between washes. One final wash with 100 uL of 1×PBS is then done to remove any residual BSA. If the plate is sealed it can be used for up to several days later.

### Forming the PAP Complex

10 mg of HRP, Sigma-Aldrich, catalogue number P6782, is dissolved in 2 mL of 1×PBS. Lyophilized rabbit anti-peroxidase antibody, Sigma-Aldrich, catalogue number P7899, is dissolved in 2.1 mL of ice cold 1×PBS and filtered using a 0.22 μm pore size 13 mm PVDF syringe filter and a 3 mL sterile syringe. The resuspended rabbit anti-peroxidase antibody is stored at 4° C. The rabbit antibody must be handed using sterile techniques to avoid bacterial contamination since the Sigma P7899 antibody does not contain a preservative agent. The resuspended antibody can be used up to one year as long as no contamination occurs as judged by an increase in turbidity. To form the PAP complex, 20 uL of the resuspended rabbit anti-peroxidase antibody is added to 950 μl of 1×PBS, followed by the addition of 50 μl of the 5 mg/mL HRP solution. The PAP complex must be used immediately. It cannot be stored, because even with antigen excess, larger immune complexes will be formed upon storage. The extreme antigen excess guarantees that any single antibody is bound to two HRP molecules and prevents larger immune complexes from forming, thus eliminating the bridging of larger immune complexes. Potential inhibitors are dissolved in 1×PBS and 100 uL of the inhibitor to be tested is then added to 100 uL of the PAP complex and allowed to incubate for 1 hour. For a control, 100 uL of 1×PBS is added to 100 uL of the PAP complex instead of the inhibitor.

### Performing the Reaction

100 uL of each inhibitor/PAP complex mixture is added to a well of the coated microwell plate as well as the control PAP complex without the added inhibitor. After allowing the plate to incubate for 1 hour, the inhibitor/PAP complex mixture is manually removed and each well is washed four more times with 100 uL of 1×PBS with no additional time between washes. 100 uL of SUB4 ABTS 1-Component HRP Microwell Substrate, ImmunoChemistry Technologies, catalogue number 6278, is added to each well and after 30 minutes of incubation, 100 uL of Stop Solution for ABTS Microwell Substrate STOP2, ImmunoChemistry Technologies, catalogue number 6283, is added to each well and the color development is read at OD 405 nm on a microwell ELISA reader or spectrophotometer.

C. Demonstration that Hepatitis a Virus Contains a FcR and that NB406 (SEQ ID NO:20) can Prevent the Binding of IC to the Hepatitis a Virus

To test whether the hepatitis A virus contained a FcR and whether NB406 (SEQ ID NO:20) could inhibit the binding of IC to the FcR of the hepatitis A virus, we used the reverse ELISA assay. Instead of binding a known FcR to the plate, such as human FcRIIa or C1q, hepatitis A virus was used. 100 uL of a 1:10 or 1:100 dilution of hepatitis A virus at a titer of 1.5×10⁶ used to coat each well of the microplate. The standard reverse ELISA protocol was implemented with the inhibitor NB406 used at a concentration of 10, 3.33 or 1.11 mg/mL. The assay was repeated three times and the average of the three replicate experiments is shown in Table 5. Hepatitis A virus is abbreviated as HAV in the table.

TABLE 5

| IC +/− | 1:10 | Percent | 1:100 | Percent |
|---|---|---|---|---|
| NB406 | HAV | Inhibition | HAV | Inhibition |
| 0 | 0.997 | | 0.953 | |
| 10 mg/mL | 0.050 | 95.0% | 0.055 | 94.2% |
| 3.33 mg/mL | 0.305 | 69.4% | 0.155 | 83.7% |
| 1.11 mg/mL | 0.762 | 23.6% | 0.774 | 18.8% |
| HRP only | 0.117 | | 0.138 | |

The results demonstrate that the hepatitis A virus contains a FcR that can bind IC and that NB406 can effectively prevent the binding of IC to the hepatitis A virus.

D. Demonstration that Hepatitis C Virus Core Protein is an FcR and that NB406 (SEQ ID NO:20) can Prevent the Binding of IC to Hepatitis C Virus Core Protein

We also tested whether the hepatitis C virus core protein was a FcR using the reverse ELISA assay. Instead of binding a known FcR to the plate, the hepatitis C virus core protein was used. The standard reverse ELISA protocol was implemented with the inhibitor NB406 used at a concentration of 10 mg/mL. The assay was repeated three times and the average of the three replicate experiments is shown in Table 6. Hepatitis C virus is abbreviated as HCV in the table.

TABLE 6

| IC +/− | 5 μg HCV | Percent |
|---|---|---|
| NB406 | Core Protein | Inhibition |
| 0 | 2.752 | |
| 10 mg/mL | 0.283 | 89.7% |
| HRP only | 0.172 | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, the materials, methods, and examples are illustrative only and not intended to be limiting.

The complete disclosures of all patents, patent applications including provisional patent applications, publications including patent publications and nonpatent publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### REFERENCES

### U.S. Patent Documents

| | | |
|---|---|---|
| 4,383,317 | May 1983 | Haville |
| 5,189,014 | February 1993 | Cowan, Jr. et al. |
| 5,693,758 | December 1997 | Gould et al. |
| 5,821,337 | October 1998 | Carter et al. |
| 5,932,483 | August 1999 | Baumann et al. |
| 6,319,897 | November 2001 | Lambris et al. |
| 6,355,683 | March 2002 | Baell et al. |
| 6,818,611 | November 2004 | Altman |
| 6,916,904 | July 2005 | Bodie et al. |
| 7,122,516 | October 2006 | Altman |
| 7,205,382 | April 2007 | Ronspeck et al. |
| 7,365,162 | April 2008 | Altman |
| 7,714,104 | May 2010 | Bodie et al. |
| 7,786,258 | August 2010 | Bodie et al. |
| 2002/0146428 | October 2002 | Hultgren et al. |
| 2003/0204050 | October 2003 | Bodie et al. |
| 2004/0087765 | May 2004 | Ronspeck et al. |
| 2004/0253247 | December 2004 | Dennis et al. |
| 2005/0148030 | July 2005 | Bodie et al. |
| 2006/0099571 | May 2006 | Altman |
| 2007/0225231 | September 2007 | Bodie et al. |
| 2007/0276125 | November 2007 | Bodie et al. |
| 2008/0187490 | August 2008 | Bodie et al. |
| 2008/0200392 | August 2008 | Bodie et al. |
| 2008/0207498 | August 2008 | Bodie et al. |
| 2009/0105138 | April 2009 | Bodie et al. |
| 2010/0113362 | May 2010 | Bodie et al. |
| 2011/0218157 | September 2011 | Bodie et al. |
| 2012/0021988 | January 2012 | Bodie et al. |
| 2012/0115791 | May 2012 | Bodie et al. |
| 2013/0130987 | May 2013 | Bodie et al. |

### Foreign Patent Documents

| | | |
|---|---|---|
| 0,097,994 | September 1987 | EP |
| 0,098,829 | May 1989 | EP |
| WO 98/26794 | June 1998 | WO |
| WO 00/22112 | April 2000 | WO |
| WO 01/45746 | June 2001 | WO |
| WO 02/38592 | May 2002 | WO |
| WO 03/091395 | November 2003 | WO |
| WO03104459 | December 2003 | WO |
| WO 2004/011485 | February 2004 | WO |
| WO2004058747 | July 2004 | WO |
| WO 2005/086947 | September 2005 | WO |
| WO2006020930 | February 2006 | WO |
| WO 2007/030475 | March 2007 | WO |
| WO 2007/068047 | June 2007 | WO |
| WO2009023777 | February 2009 | WO |

### Ebola References

“Ebola Outbreaks and Research” AbD Serotec-A Bio-Rad Company; recovered at <static.abdserotec.com/2014/ebola/ebola-infographic.pdf> on Feb. 18, 2015.
Abdalla et al. “The burden of malaria in Sudan: incidence, mortality and disability-adjusted life-years” Malaria Journal, 2007 July; 6:97.
Abo et al. “Biology of autoreactive extrathymic T cells and B-1 cells of the innate immune system” ImmunolRes. 2012 June; 53(3):224-230.
Abu-Raddad et al. “Genital herpes has played a more important role than any other sexually transmitted infection in driving HIV prevalence in Africa” PLoS One, 2008 May; 3(5): e2230.
Abu Raddad et al., “Dual infection with HIV and malaria fuels the spread of both diseases in sub Saharan Africa” Science, 2006 December; 314(5805):1603-6.
Adam et al., “Cryoglobulins, Circulating Immune Complexes, and Complement Activation in Cerebral Malaria” Infection and Immunity, 1981; 31(2):530-535.
Adjei et al., “Seroprevalence of HHV-8 CMV, and EBV among the general population in Ghana West Africa” BMC Infectious Diseases, 2008 August; 8:111.
Ahmed et al. “Hepatitis C virus infection in vitro triggers endoplasmic reticulum stress and downregulates insulin receptor substrates 1 and 2 through upregulation of cytokine signaling suppressor 3” Acta Virol 2014 September; 58(3):238-44.
Alexander et al. “Circulating immune complexes in cynomolgus macaques” Lab Anim Sci 1985 October; 35(5):465-8.
Ali et al., “Immunologic Aspects of the Pathogenesis of Human Onchocerciasis” Doctoral Thesis from the Department of Immunology, Stockhom, Sweden, 2006; 1-68; Recovered from the internet on Feb. 18, 2015<http://www.diva-portal.org/smash/get/diva2:199307/FULLTEXT01.pdf>
Allela et al., “Ebola Virus antibody Prevalence in Dogs and Human Risk” Emerging Infectious Diseases, 2005 March; 11(3):358-390.
American Museum of Natural History. “Scientists find soaring variety of malaria parasites in bats.” ScienceDaily. ScienceDaily, 7 Oct. 2013. <www.sciencedaily.com/releases/2013/10/131007151729.htm>.
Amman et al., “Seasonal Pulses of Marburg Virus circulation in Juvenile Rousettus aegyptiacus Bats Coincide with Periods of Increased Risk of Human Infection” PLoS Pathogens, October 2012; 8(10):e1002877.
Amuasi et al., “Access to Artemisinin-Combination Therapy (ACT) and other Anti-Malarials: National Policy and markets in Sierra Leone” PLoS One, October 2012; 7(10):e47733.
Anderson et al., “Pathogenesis of B Cell Superantigen-induced Immune Complex-Mediated Inflammation” Infection and Immunity, February 2006; 74(2): 1196-1203
Ansari, “Clinical features and pathobiology of Ebolavirus infection” Journal ofAutoimmunity, 2014 December; 55:1-9. Published Online 2014 Sep. 26.
Anthony et al., “Identification of a receptor required for the anti-inflammatory activity of IVIG” PNAS USA, Dec. 16 2008; 105(50):19571-19578.
Antonssont et al., “Binding of human and animal immunoglobulins to the IgG Fc receptor induced by human cytomegalovirus” Journal of General Virology, 2001; 82(Pt. 5):1137-1145.
Apata et al., “Progress toward Prevention of Transfusion-Transmitted Hepatitis B and Hepatitis C Infection—Sub-Saharan Africa, 2000-2011” Morb Mortal Wkly Rep, 2014, July 25; 63(29):613-9.
Arama et al., “Interethnic Differences in Antigen-Presenting Cell Activation and TLR Responses in Malian children during Plasmodium falciparum malaria” PLoS One, 2011 March; 6(3):e18319.
Ardiet et al., “Patterns of malaria indices across three consecutive seasons in children in a highly endemic area of West Africa: a three times-repeated cross-sectional study” Malaria Journal, 2014, May 28; 13:199.
Arendse et al., “The acute, the chronic and the news of HIV-related renal disease in Africa” Kidney Int, 2010 August; 78(3):239-45.
Arinola et al., “Leucocyte Phagocytosis and circulating immune complexes in mothers after child birth” West Afr J Med, 2004 July-September; 23(3):256-9.
Armour et al., “The contrasting IgG-binding interactions of human and herpes simplex virus Fc receptors” Biochem Soc Trans, 2002 August; 30(4):495-500.
Arruda et al., “Heme Inhibits Human Neutrophil Apoptosis: Involvement of Phosphoinositide 3-Kinase, MAPK, and NF-kappaB” J Immunol, 2004 August; 173(3):2023-2030.
Astoul et al., “Rabies Superantigen as a Vbeta T-dependent Adjuvant” J Exp Med, 1996 April; 183(4):1623-1631.
Audet et al., “Molecular Characterization of the Monoclonal Antibodies Composing ZMAb: A Protective Cocktail Against Ebola Virus” Scientific Reports, 2014 Nov. 6; 4:6881.
Ayithan et al. “Ebola virus-like particles stimulate type I interferons and proinflammatory cytokine expression through the toll-like receptor and interferon signaling pathways” J Interferon cytokine Res, 2014 February; 34(2):79-89.
Baize et al. “Letter to the Editor: Evidence for Ebola Virus Superantigen Activity” Journal of Virology, 2011 April; 85(8): 4041-4042.
Baize et al. “Early and Strong Immune Responses are Associated with Control of Viral Replication and Recovery in Lassa Virus-Infected Cynomolgus Monkeys” Journal of Virology, 2009 June; 83(11):5890-5903.
Baize et al., Emergence of Zaire Ebola Virus Disease in Guinea, N Engl J Med. 2014 Oct. 9; 371(15):1418-25
Baize et al. “Emergence of Zaire Ebola Virus Disease in Guinea Preliminary Report” The New England Journal of Medicine, 2014 Apr. 25; 8 pages.
Baker et al., “The Role of FcRn in Antigen Presentation” Frontiers in Immunology, prepublished online 2014 Jul. 18; Vol. 5: Article 408, 12 pages.
Bale et al. “Structural Basis for Differential Neutralization of Ebolaviruses” Viruses 2012 April; 4(4):447-470.
Baribaud et al. “The role of DC-SIGN and DC-SIGNR in HIV and Ebola virus infection: can potential therapeutics block virus transmission and dissemination?” Expert Opin. Ther. Targets, 2002 August; 6(4):423-431.
Barrette et al., “Discovery of Swine as a Host for the Reston ebolavirus” Science 2009; 325:204-6.
Batten et al., “TNF Deficiency Fails to Protect BAFF Transgenic Mice against Autoimmunity and Reveals a Predisposition to B Cell Lymphoma” J Immunol, 2004; 172(2):812-822.
Becker, “HIV-peplotion vaccine—a novel approach to protection against AIDS by transepithelial transport of viral peptides to Langerhans cells for long-term antiviral CTL response (A review)” Acta Microbiol Immunol Hung, 1996; 43(1): 1-17.
Becker, “HIV-1 proteins in infected cells determine the presentation of viral peptides by HLA class I and class II molecules and the nature of the cellular and humoral antiviral immune responses—a review” Virus Genes, 1994 July; 8(3)249-270.
Becker, “Dengue fever virus and Japanese encephalitis virus synthetic peptides, with motifs to fit HLA class I haplotypes prevalent in human populations in endemic regions, can be used for application to skin Langerhans cells to prime antiviral CD8+ cytotoxic T cells (CTLs)—a novel approach to the protection of humans” Virus Genes, 1994 September; 9(1):33-45.
Becker, “Respiratory syncytial virus RSV evades the human adaptive immune system by skewing the Th1/Th2 cytokine balance toward increased levels of Th2 cytokines and IgE, markers of allergy—a review” Virus Genes, 2006 October; 33(2):235-252.
Becker, “Respiratory syncytial virus(RSV)-induced allergy may be controlled by IL-4 and CX3C fractalkine antagonists and CpG ODN as adjuvant: hypothesis and implications for treatment” Virus Genes, 2006; 33(2):253-264.
Becker, “An analysis of the role of skin Langerhans cells (LC) in the cytoplasmic processing of HIV-1 peptides after “peplotion” transepidermal transfer and HLA class I presentation to CD8+ CTLs—an approach to immunization of humans” Virus Genes, 1995 January; 9(2):133-47.
Becquart et al. “High prevalence of both humoral and cellular immunity to Zaire ebolavirus among rural populations in Gabon” PLoS One, 2010 Feb. 9; 5(2):e9126.
bei Resources, “Product Information Sheet for NR-12208—Monoclonal Anti-Sudan Ebolavirus Envelope Glycoprotein, Clone 6D11 (produced in vitro)” 2014.
Bellan et al., “Ebola control: effect of asymptomatic infection and acquired immunity” Lancet 2014 Oct. 25; 384(9953):1499-500.
Bello et al. “Cytomegalovirus infection in Gambian mothers and their babies” J Clin Pathol, 1991 May; 44(5):366-369.
Bermejo et al. “Ebola Outbreak Killed 5000 Gorillas” Science, 2006 Dec. 8; 314(5805):1564.
Bielefeldt-Ohmann, “Analysis of antibody-independent binding of dengue viruses and dengue virus envelope protein to human myelomonocytic cells and B lymphocytes” Virus Research, 1998 September; 57(1):63-79.
Blewett et al., “Isolation and characterization of an endogenous cytomegalovirus (BaCMV) from baboons” Arch Virol, 2001; 146(9):1723-38.
Blewett et al. “Isolation of cytomegalovirus and foamy virus from the drill monkey (Mandrillus leucophaeus) and prevalence of antibodies to these viruses amongst wild-born and captive-bred individuals” Arch Virol, 2003 March; 148(3):423-33.
Bonnet et al. “Prevalence of cryoglobulinemia and serological markers of autoimmunity in human immunodeficiency virus infected individuals: a cross-sectional study of 97 patients” The Journal of Rheumatology Sep. 1 2003 vol 30 No 9 2005 2010
Boonnak et al. “Human FcγRII cytoplasmic domains differentially influence antibody-mediated dengue virus infection” J Immunol, 2013 Jun. 1; 190(11):5659-65.
Boonnak et al. “Role of Dendritic Cells in Antibody-Dependent Enhancement of Dengue Virus Infection”) J Virol, Published Online 2008 Feb. 13; 82(8):3939-3951.
Boruchov et al. “Activating and inhibitory IgG Fc receptors on human DCs mediate opposing functions” Journal of Clin Invest, Published Online 2005 Sep. 15; 115(10):2914-23.
Bostrom et al. “Changes in the levels of cytokines, chemokines and malaria-specific antibodies in response to Plasmodium falciparum infection in children living in sympatry in Mali” Malaria Journal, 2012 Apr. 5; 11:109.
Boudin et al. “Plasmodium falciparum and P. malariae epidemiology in a West African village” Bull Word Health Organ, 1991; 69(2):199-205.
Branco et al. “Characterization of the Lassa virus GP1 ectodomain shedding: implications for improved diagnostic platforms” Virology Journal, 2009 Sep. 24; 6:147.
Branco et al. “Shedding of soluble glycoprotein 1 detected during acute Lassa virus infection in human subjects” Virology Journal, 2010 Nov. 9; 7:306
Bray, “Viral Hemorrhagic Fever (Crimean-Congo, Ebola, Lassa, Marburg, Rift Valley, Yellow Fever)” Available online at <http://www.antimicrobe.org/v39.asp>
Breman et al. “Conquering the intolerable burden of malaria: what's new, what's needed: a summary” Am J Trop Med Hyg, 2004 August; 71(2 Suppl):1-15.
Bukreyev et al. “The secreted form of respiratory syncytial virus G glycoprotein helps the virus evade antibody-mediated restriction of replication by acting as an antigen decoy and through effects on Fc receptor-bearing leukocytes” Journal of Virology, Published Online 2008, Oct. 8; 82(24):12191-204.
Burova et al. “Role of streptococcal IgG Fc receptor in tissue deposition of IgG in rabbits immunized with Streptococcus pyogenes” APMIS, 1992 June; 100(6):567-74.
Busico et al. “Prevalence of IgG antibodies to Ebola virus in individuals during an Ebola outbreak, Democratic Republic of the Congo, 1995” J Infect Dis, 1999 February; 179 Suppl 1:S102-7.
Butler et al. “Antibody repertoire development in fetal and neonatal piglets: XIX. Undiversified B cells with hydrophobic HCDR3s preferentially proliferate in the porcine reproductive and respiratory syndrome” J Immunol, 2007 May 15; 178(10):6320-6331.
Butler et al., “Porcine reproductive and respiratory syndrome virus subverts repertoire development by proliferation of germline-encoded B cells of all isotypes bearing hydrophobic heavy chain CDR3” J Immunol, 2008 Feb. 15; 180(4):2347-2356.
Calif Inst of Tech, “Why Anti HIV Antibodies are ineffective at Blocking Infection” Science Daily, 2009 Apr. 30, <www.sciencedaily.com/releases/2009/04/090422151832.htm>
Calisher et al., “Bats: Important Reservoir Hosts of Emerging Viruses” Cinical Microbiology Rev, 2006 July; 19(3):531-545.
Campbell et al. “Therapeutic effect of IVIG on inflammatory arthritis in mice is dependent on the Fc portion and independent of sialylation or basophils” Journal of Immunology, 2014 Jun. 1; 192(11):5031-5038.
The Center for Food Security and Public Health, Iowa State University, College of Veterinary Medicine “Ebolavirus and Marburgvirus Infections” 2014 December; 13 pages. <http://www.cfsph.iastate.edu/Factsheets/pdfs/viral_hemorrhagic_fever_filovirus.pdf>
Centers for Disease Control and Prevention (CDC), “Imported Case of Marburg Hemorrhagic Fever—Colorado 2008” MMWR Morb Mortal Wkly Rep, 2009 Dec. 18; 58(49):1377-1381.
Chan et al., “Ligation of Fc gamma receptor IIB inhibits antibody-dependent enhancement of dengue virus infection” PNAS USA, Published Online 2011 Jul. 11; 108(30):12479-12484.
Chandran et al., “Recurrent HIV-associated immune complex glomerulonephritis with lupus-like features after kidney transplantation” Am J Kidney Dis, 2013 August; 62(2):335-338.
Chandrashekar et al., “Circulating immune complex-associated parasite antigens in human onchocerciasis” J Infect Dis, 1990 November; 162(5):1159-1164.
Chareonsirisuthigul et al. “Dengue virus (DENV) antibody-dependent enhancement of infection upregulates the production of anti-inflammatory cytokines, but suppresses anti-DENV free radical and pro-inflammatory cytokine production, in THP-1 cells” Journal of General Virology, 2007 February; 88(Pt 2):365-375.
Chawla et al. “Dengue virus neutralization in cells expressing Fc gamma receptors” PLoS One, 2013 May 22; 8(5):e65231.
Chen et al. “HCV and HIV co-infection: mechanisms and management” Nat Rev Gastroenterology Hepatol, 2014 June; 11(6):362-371.
Chen et al., “Binding of HIV-1 gp120 to DC-SIGN promotes ASK-1-dependent activation-induced apoptosis of human dendritic cells” PLoS Pathog, Published Online 2013 Jan. 31; 9(1):e1003100.
Chippaux, “Outbreaks of Ebola virus disease in Africa: the beginnings of a tragic saga” Journal Venom Anim Toxins Incl Trop Dis, 2014 Oct. 3; 20(1):44.
Chuang et al., “Re-evaluation of the pathogenic roles of nonstructural protein 1 and its antibodies during dengue virus infection” Journal of Biomedical Science, 2013 Jun. 27; 20:42.
Chuang et al. “Molecular mimicry between dengue virus and coagulation factors induces antibodies to inhibit thrombin activity and enhance fibrinolysis” J Virol, Accepted Manuscript posted online 2014 Sep. 17; 88(23):13759-68.
Connolly et al. “Pathogenesis of Experimental Ebola Virus Infection in Guinea Pigs” J Infect Dis, 1999 February; 179(Suppl 1):S203-17.
Connor et al. “Fc receptors for IgG (Fc gamma Rs) on human monocytes and macrophages are not infectivity receptors for human immunodeficiency virus type 1 (HIV-1): studies using bispecific antibodies to target HIV-1 to various myeloid cell surface molecules, including the Fc gamma R” Proc Natl Acad Sci USA, 1991 Nov. 1; 88(21):9593-7.
Corey et al., “The effects of herpes simplex virus-2 on HIV-1 acquisition and transmission: a review of two overlapping epidemics” J Acquir Immune Defic Syndr, 2004 Apr. 15; 35(5):435-445.
Corrales-Aguilar et al., “A novel assay for detecting virus-specific antibodies triggering activation of Fcγ receptors” J Immunol Methods, Published Online 2012 Sep. 27; 387(1-2):21-35.
Corrales-Aguilar et al., “Human cytomegalovirus Fcγ binding proteins gp34 and gp68 antagonize Fcγ receptors I, II and III” PLoS Pathogens, 2014 May 15; 10(5):e1004131.
Cosio et al. “Clearance of human antibody/DNA immune complexes and free DNA from the circulation of the nonhuman primate” Clin Immunol Immunopathol, 1987 January; 42(1):1-9.
Cosio et al., “Role of fibronectin on the clearance and tissue uptake of antigen and immune complexes in rats” J. Cln Invest, 1987 November; 80(5): 1270-9.
Csomor et al., “Complement protein C1q induces maturation of human dendritic cells” Mol Immunol, Published Online 2007 Mar. 26; 44(13):3389-97.
D'Elia et al., “Targeting the “cytokine storm” for therapeutic benefit” Clin Vaccine Immunol, Published Online 2013 Jan. 2; 20(3):319-27.
Dauer et al., “Interferon-alpha disables dendritic cell precursors: dendritic cells derived from interferon-alpha-treated monocytes are defective in maturation and T-cell stimulation” Immunology, 2003 September; 110(1):38-47.
De Benedetti et al., “Interleukin 8 and monocyte chemoattractant protein-1 in patients with juvenile rheumatoid arthritis. Relation to onset types, disease activity, and synovial fluid leukocytes” J Rheumatol, 1999 February; 26(2):425-31.
de Oliveira et al. “Increased survival in B-cell-deficient mice during experimental cerebral malaria suggests a role for circulating immune complexes” MBio 2014 March; 5(2):e00949-14.
De Re et al., “Sequence analysis of the immunoglobulin antigen receptor of hepatitis C virus-associated non-Hodgkin lymphomas suggests that the malignant cells are derived from the rheumatoid factor-producing cells that occur mainly in type II cryoglobulinemia” Blood, 2000 Nov. 15; 96(10):3578-84.
Delerue et al., “Inducible, tightly regulated and non-leaky neuronal gene expression in mice” Transgenic Res, 2014 April; 23(2):225-33.
Dias et al., “A shared structural solution for neutralizing ebolaviruses” Nat Struct Mol Biol, 2011 Nov. 20; 18(12):1424-1427.
Diebolder et al., “Complement is activated by IgG hexamers assembled at the cell surface” Science, 2014 Mar. 14; 343(6176):1260-3.
Ding et al., “Suppressor of cytokine signaling 1 inhibits IL-10-mediated immune responses” J Immunol, 2003 Feb. 1; 170(3):1383-91.
Donati et al., “Increased B cell survival and preferential activation of the memory compartment by a malaria polyclonal B cell activator” J Immunol, 2006 Sep. 1; 177(5):3035-3044.
Dowd et al., “Antibody-mediated neutralization of flaviviruses: a reductionist view” Virology, 2011 Mar. 15; 411(2):306-315—Published Online 2011 Jan. 20.
Drummer, “Challenges to the development of vaccines to hepatitis C virus that elicit neutralizing antibodies” Front Microbiol, 2014 Jul. 3; 5:329.
Ebihara et al., “Host response dynamics following lethal infection of rhesus macaques with Zaire ebolavirus” The Journal of Infectious Diseases, 2011 Nov. 1; 204(Suppl 3):S991-S999.
Ellaurie et al., “Correlation of serum antigen and antibody concentration with clinical features in HIV infection” Archives of Disease In Childhood, 1991 February; 66(2):200-3.
Elshafie et al., “Circulating immune complexes (IC) and IC-induced levels of GM-CSF are increased in sudanese patients with acute visceral Leishmania donovani infection undergoing sodium stibogluconate treatment: implications for disease pathogenesis” J Immunol, 2007 Apr. 15; 178(8):5383-5389.
Endy, “Human immune responses to dengue virus infection: lessons learned from prospective cohort studies” Frontiers in Immunology, 2014 Apr. 24; 5:183.
Falconar et al., “The NS1 glycoprotein can generate dramatic antibody-enhanced dengue viral replication in normal out-bred mice resulting in lethal multi-organ disease” PLoS One, 2011; 6(6):e21024.
Falugi et al., “Role of protein A in the evasion of host adaptive immune responses by Staphylococcus aureus” MBio, 2013 Aug. 27; 4(5):e00575-13.
Falzarano et al., “Vaccines for Viral Hemorrhagic Fevers-Progress and Shortcomings” Curr Opin Virol, 2013 June; 3(3):343-351.
Feldmann et al., “Emerging and reemerging of filoviruses” Arch Virol Suppl., 1996; 11:77-100.
Feldmann et al. “Ebola Haemorrhagic fever” Lancet, 2011 Mar. 5; 377(9768):849-862.
Feldmann, “Ebola A Growing Threat” The New England Journal of Medicine, 2014 Oct. 9; 371(15):1375-8
Fernandez-Arias et al., “Malaria inhibits surface expression of complement receptor 1 in monocyte macrophages causing decreased immune complex internalization” J Immunol, 2013 April 1; 190(7):3363-3372.
Fiotti et al. “Atherosclerosis and inflammation Patterns of cytokine regulation in patients with peripheral arterial disease” Atherosclerosis, 1999 July; 145(1):51-60.
Fischer et al. “Hepatitis C virus infection and apoptosis” World J Gastroenterology, 2007 Sep. 28; 13(36):4865-4872.
Fiscus et al. “Infectious immune complexes in HIV-1-infected patients” Viral Immunol, 1993 Summer, 6(2):135-41.
Fletcher et al. “Development of Nephritis but not Sialadenitis in autoimmune-prone BAFF transgenic mice lacking marginal zone B cells” Eur J Immunol, 2006 September; 36(9):2504-14.
Fletcher et al. “Development of autoimmune nephritis in genetically asplenic and splenectomized BAFF transgenic mice” J Autoimmun, 2011 March; 36(2): 125-34.
Flick et al., “Role of Nonimmune IgG Bound to PfEMP1 in Placental Malaria” Science, 2001 Sep. 14; 293(5537):2098-100.
Foy et al. “Comparison of Risk Factors and Outcomes in HIV Immune Complex Kidney Disease and HIV-Associated Nephropathy” Clin J Am Soc Nephrol, 2013 September; 8(9): 1524-1532. Published Online 2013, May 16.
Francica et al. “Steric Shielding of Surface Epitopes and Impaired Immune Recognition Induced by the Ebola Virus Glycoprotein” PLoS Pathogens, 2010 Sep. 9; 6(9):e1001098.
Füst, “Enhancing antibodies in HIV infection” Parasitology, 1997; 115 Suppl:S127-40.
Gagnon et al., “T Cell Receptor Vbeta Gene Usage in Thai Children With Dengue Virus Infection” Am J Trop Med Hyg, 2001 January-February; 64(1-2):41-48.
Gallagher, “Ebola: Are cases levelling off?” BBC News Health, 2014 Nov. 1, Available online at <http://www.bbc.com/news/health-29847058>.
Garcia et al., “Asymptomatic dengue infection in a Cuban population confirms the protective role of the RR variant of the FcgammaRIIa polymorphism” Am J Trop Med Hyg, 2010 June; 82(6):1153-1156.
Garske et al., “Estimating Air Temperature and its Influence on Malaria Transmission Across Africa” PLoS One, 2013; 8(2):e56487
Gatherer, “The 2014 Ebola virus disease outbreak in West Africa” Journal of Gen Virology, 2014 August; 95(Pt 8):1619-24.
Geiger et al., “Declining malaria parasite prevalence and trends of asymptomatic parasitaemia in a seasonal transmission setting in North-Western Burkina Faso between 2000 and 2009-2012” Malaria Journal, 2013 Jan. 22; 12:27.
Geisbert et al., “Pathogenesis of Ebola hemorrhagic fever in cynomolgus macaques: evidence that dendritic cells are early and sustained targets of infection” American Journal of Pathology, 2003 December; 163(6):2347-70.
Geisbert et al., “Pathogenesis of Ebola hemorrhagic fever in primate models: evidence that hemorrhage is not a direct effect of virus-induced cytolysis of endothelial cells” American Journal of Pathology, 2003 December; 163(6):2371-82.
Geimonen et al., “Pathogenic and nonpathogenic hantaviruses differentially regulate endothelial cell responses” PNAS USA, 2002 Oct. 15; 99(21):13837-42. Published Online 2002 Oct. 4.
Ghebrehiwet et al., “The C1q family of proteins: insights into the emerging non-traditional functions” Front Immunol, 2012 Apr. 5; 3:52.
Gibson et al., “Treating cofactors can reverse the expansion of a primary disease epidemic” BMC Infect Dis, 2010 Aug. 23; 10:248.
Gillrie et al., “Plasmodium falciparum histones induce endothelial proinflammatory response and barrier dysfunction” Am J Pathol, 2012 March; 180(3):1028-39. Published Online 2012 Jan. 17.
Gimenez et al., “Neutralizing and enhancing activities of human respiratory syncytial virus-specific antibodies” Clinical and Diagnostic Laboratory Immunology, 1996 May; 3(3):280-6.
Gire et al., “Genomic surveillance elucidates Ebola virus origin and transmission during the 2014 outbreak” Science, 2014 Sep. 12; 345(6202):1369-72. Published Online 2014 Aug. 28.
Gneme et al. “Plasmodium species occurrence, temporal distribution and interaction in a child-aged population in rural Burkina Faso” Malaria Journal, 2013 Feb. 19; 12:67.
Goldfield, “Ebola and Malaria: An update on Sierra Leone” 2014 Aug. 14; <http://www.nothingbutnets.net/blogs/ebola-and-malaria-an-update-sierra-leone.html>.
Gomes et al., “Autoimmunity, phospholipid-reacting antibodies and malaria immunity” Lupus, 2014 October; 23(1):1295-8.
Goncalvez et al., “Monoclonal antibody-mediated enhancement of dengue virus infection in vitro and in vivo and strategies for prevention” PNAS USA, 2007 May 29; 104(22): 9422-7. Published Online 2007 May 15.
Gonzalez et al., “Ebola virus circulation in Africa: a balance between clinical expression and epidemiological silence” Bull Soc Pathol Exot, 2005 September; 98(3):210-7.
González-Hernández et al., “Human adrenal cells express tumor necrosis factor-alpha messenger ribonucleic acid: evidence for paracrine control of adrenal function” J Clin Endocrinol Metab, 1996 February; 81(2):807-13.
Goodglick et al., “Revenge of the Microbes. Superantigens of the T and B Cell Lineage” American Journal of Pathology, 1994 April; 144(4):623-36.
Goodman et al., “Cost-effectiveness of malaria control in sub-Saharan Africa” Lancet, 1999 Jul. 31; 354(9176):378-85.
Goodyear et al., “Cutting Edge: Bim is required for superantigen-mediated B cell death” J Immunol, 2007; 178(5):2636-2640.
Graille et al., “Crystal structure of a Staphylococcus aureus protein A domain complexed with the Fab fragment of a human IgM antibody: structural basis for recognition of B-cell receptors and superantigen activity” PNAS USA, 2000; 97(10):5399-5404.
Grard et al., “Emergence of divergent Zaire ebola virus strains in Democratic Republic of the Congo in 2007 and 2008” J Infect Dis, 2011 November; 204 Suppl 3:S776-84.
Groseth et al., “The ecology of Ebola virus” Trends Microbiol, 2007 September; 15(9):408-16.
Guadagnoli et al., “Development and characterization of April antagonistic monoclonal antibodies for treatment of B cell lymphomas” Blood, 2011; 117(25):6856-65.
Gupta et al., “Passive transfer of antibodies protects immunocompetent and imunodeficient mice against lethal Ebola virus infection without complete inhibition of viral replication” Journal of Virology, 2001; 75(10):4649-54.
Gupta et al., “Persistent Infection with Ebola virus under Conditions of Partial Immunity” Journal of Virology, 2004 January; 78(2):958-67.
Gupta et al., “Serology and cytokine profiles in patients infected with the newly discovered Bundibugyo ebolavirus” Virology, 2012; 423(2):119-24.
Guzman et al. “Secondary Infection as a risk factor for dengue hemorrhagic fever/dengue shock syndrome: an historical perspective and role of antibody-dependent enhancement of infection” Arch Virol, 2013 July; 158(7):1445-59.
Guzman et al., “The Complexity of Antibody-Dependent Enhancement of Dengue Virus Infection” Viruses, 2010; 2(12):2649-62.
Halstead et al., “Intrinsic antibody-dependent enhancement of microbial infection in macrophages: disease regulation by immune complexes” Lancet Infect Dis, 2010 October; 10(10):712-22.
Halstead, “Controversies in dengue pathogenesis” Paediatr Intl Child Health, 2012; 32 Suppl 1:5-9.
Hart et al., “Immune complexes bind preferentially to Fc gamma RIIA (CD32) on apoptotic neutrophils, leading to augmented phagocytosis by macrophages and release of proinflammatory cytokines” J Immunol, 2004; 172(3):1882-7.
Hartgers et al., “Enhanced Toll-like receptor responsiveness associated with mitogen-activated protein kinase activation in Plasmodium falciparum-infected children” Infection and Immunity, 2008; 76(11):5149-57.
Hay et al., “A World Malaria Map: Plasmodium falciparum Endemicity in 2007” PLoS Medicine, 2009; 6(3):e1000048.
Heap et al., “Analysis of a 17-amino acid residue, virus-neutralizing microantibody” Journal of General Virology, 2005; 86(Pt 6):1791-1800.
Heffernan et al., “Low seroprevalence of IgG antibodies to Ebola virus in an epidemic zone: Ogooue-Ivindo region, Northeastern Gabon, 1997” J Infect Dis, 2005; 191(6):964-8.
Helegbe et al., “Anti-erythropoietin antibody levels and its association with anaemia in different strains of semi-immune mice infected with Plasmodium berghei ANKA” Malaria Journal, 2013 August; 12:296.
Hensley et al., “Pathogenesis of Lassa fever in cynomolgus macaques” Virology Journal, 2011; 8:205.
Hofmann-Winkler et al., “Host cell factors in filovirus entry: novel players, new insights” Viruses, 2012 December; 4(12):3336-62.
Hohdatsu et al., “The role of IgG subclass of mouse monoclonal antibodies in antibody-dependent enhancement of feline infectious peritonitis virus infection of feline macrophages” Arch Virol, 1994; 139(3-4):273-85.
Horakova et al., “Complement mediates the binding of HIV to erythrocytes” J Immunol, 2004; 173(6):4236-41.
Hosszu et al., “DC-SIGN, C1q, and gC1qR form a trimolecular receptor complex on the surface of monocyte-derived immature dendritic cells” Blood, 2012; 120(6):1228-36.
Hu et al., “Human immune responses to Plasmodium falciparum infection: molecular evidence for a suboptimal THαβ and TH17 bias over ideal and effective traditional TH1 immune response” Malaria Journal, 2013; 12:392.
Huber et al., “Emerging role for complement in HIV infection” Curr Opin HIV AIDS, 2011 September; 6(5):419-26.
Hunt et al., “Filovirus Entry: A Novelty in the Viral Fusion World” Viruses, 2012; 4(2):258-75.
Hutchinson et al., “Cytokine and chemokine expression in humans infected with Sudan Ebola virus” J Infect Dis, 2007; 196 Suppl 2:S357-63.
Iankov et al. “Neutralization capacity of measles virus H protein specific IgG determines the balance between antibody-enhanced infectivity and protection in microglial cells” Virus Res 2013 March; 172(1-2):15-23.
IBT Bioservices, “Anti-ZEBOV GP murine/human chimeric monoclonal antibody (c13C6 FR1)” <https://ibtbioservices.com/index.php/reagent-store/product/download/file_id-i151>
Illick et al., “Uncoupling GP1 and GP2 expression in the Lassa virus glycoprotein complex: implications for GP1 ectodomain shedding” Virology Journal, 2008; 5:161.
Indik et al., “The molecular dissection of Fc gamma receptor mediated phagocytosis” Blood, 1995; 86(12):4389-99.
Iriemenam et al., “Cytokine profiles and antibody responses to Plasmodium falciparum malaria infection in individuals living in Ibadan, southwest Nigeria” Aft Health Sci, 2009; 9(2):66-74.
Isenberg et al., “Efficacy and safety of atacicept for prevention of flares in patients with moderate-to-severe systemic lupus erythematosus (SLE): 52-week data (APRIL-SLE randomised trial)” Ann Rheum Dis, Published Online 2014 Jun. 20.
Islam et al., “Changes in the peripheral blood T-Cell receptor V beta repertoire in vivo and in vitro during shigellosis” Infect Immun, 1996 April; 64(4):1391-9.
Israelsson et al., “Differences in Fcgamma receptor IIa genotypes and IgG subclass pattern of anti-malarial antibodies between sympatric ethnic groups in Mali” Malaria Journal, 2008; 7:175.
Iwasa et al., “sGP serves as a structural protein in Ebola virus infection” J Infect Dis, 2011; 204 Suppl 3:S897-903.
Jack et al. “Health Investments and Economic Growth: Macroeconomic Evidence and Microeconomic Foundations” Policy Research Working Paper—4877, 2009, March; Available Online at <https://openknowledge.worldbank.rg/bitstream/handle/10986/4072/WPS4877.pdfsequence=1
Jahrling et al., “Pathogenesis of Lassa virus infection in guinea pigs” Infection and Immunity, 1982; 37(2):771-8.
Jahrling, “Protection of Lassa virus-infected guinea pigs with Lassa-immune plasma of guinea pig, primate, and human origin” J Med Virol, 1983; 12(2):93-102.
Jahrling et al., “Passive antibody therapy of Lassa fever in cynomolgus monkeys: importance of neutralizing antibody and Lassa virus strain” Infection and Immunity, 1984; 44(2):528-33.
James et al., “Structural basis for PRYSPRY-mediated tripartite motif (TRIM) protein function” PNAS USA, 2007; 104(15):6200-5.
Jans et al., “Fc gamma receptors in respiratory syncytial virus infections: implications for innate immunity” Rev Med Virol, 2014 January; 24(1):55-70. Published Online 2013 Nov. 14.
Jarvis et al., “In vitro induction of proinflammatory cytokine secretion by juvenile rheumatoid arthritis synovial fluid immune complexes” Arthritis Rheum, 1997; 40(11):2039-46.
Jarvis et al., “Immune complex size and complement regulate cytokine production by peripheral blood mononuclear cells” Clin Immunol 1999; 93(3):274-82.
Jaume et al., “Anti-severe acute respiratory syndrome coronavirus spike antibodies trigger infection of human immune cells via a pH- and cysteine protease-independent FcγR pathway” Journal of Virology, 2011; 85(20): 10582-97.
Jiang et al., “T cell activation by soluble C1q-bearing immune complexes: implications for the pathogenesis of rheumatoid arthritis” Clin Exp Immunol, 2003; 131(1):61-7.
Jiang et al., “Cord blood and adult T cells show different responses to C1q-bearing immune complexes” Cell Immunol, 2004; 229(1):62-7.
Johansson et al., “Studies of protein A and herpes simplex virus-1 induced Fc gamma-binding specificities. Different binding patterns for IgG3 from Caucasian and Oriental subjects” Immunology, 1994; 83(4):631-8.
Johnson et al. “Pyridinyl imidazole inhibitors of p38 MAP kinase impair viral entry and reduce cytokine induction by Zaire ebolavirus in human dendritic cells” Antiviral Res, 2014 July; 107:102-9. Published Online 2014 May 9.
Juompan et al., “Selective deficit in antibodies specific for the superantigen binding site of gp120 in HIV infection” FASEB J, 1998; 12(14):1473-80.
Kapoor et al., “Effects of preformed immune complexes on liver enzymes and their serum clearance in mice” Indian J Med Res, 1991; 94:222-7.
Karray et al., “Structural Basis of the gp120 Superantigen-Binding Site on Human Immunoglobulins” J Immunol, 1998; 161(12):6681-8.
Kedzierski et al., “Suppressor of cytokine signaling 4 (SOCS4) protects against severe cytokine storm and enhances viral clearance during influenza infection” PLoS Pathogens, 2014 May 8; 10(5):e1004134.
Kieny, “List of Publications” Health Systems and Innovation World Health Organization, as of 2014 June 15<http://www.who.int/dg/adg/Kieny_MP_Publications.pdf>
Kim et al., “Protein A-specific monoclonal antibodies and prevention of Staphylococcus aureus disease in mice” Infect Immun, 2012 October; 80(10):3460-70.
Klein et al., “Examination of the contributions of size and avidity to the neutralization mechanisms of the anti-HIV antibodies b12 and 4E10” PNAS USA, 2009; 106(18):7385-90.
Klingstrom et al., “Passive immunization protects cynomolgus macaques against Puumala hantavirus challenge” Antivir Ther, 2008; 13(1):125-33.
Kobinger et al., “Replication, pathogenicity, shedding, and transmission of Zaire ebolavirus in pigs” J Infect Dis, 2011; 204(2):200-8.
Konishi et al., “A simple assay system for infection-enhancing and -neutralizing antibodies to dengue type 2 virus using layers of semi-adherent K562 cells” J Virol Methods, 2010; 163(2):360-7.
Koons et al., “Ebola Drug from Japan May Emerge Among Key Candidates” Bloomberg News 2014 August 7; <http://www.bloomberg.com/news/articles/2014-08-07/ebola-drug-from-japan-may-emerge-among-key-candidates>
Koraka et al., “Detection of immune-complex-dissociated nonstructural-1 antigen in patients with acute dengue virus infections” Journal of Clinical Microbiology, 2003; 41(9):4154-9.
Kosugi et al., “Endocytosis of soluble IgG immune complex and its transport to lysosomes in hepatic sinusoidal endothelial cells” J Hepatol, 1992; 16(1-2):106-14.
Krafp et al., “Circulating immune complexes in HIV-infected persons” Klin Wochenschr, 1990; 68(6):299-305.
Krakauer, “Update on staphylococcal superantigen-induced signaling pathways and therapeutic interventions” Toxins (Basel), 2013 Sep. 24; 5(9):1629-54.
Krebs et al., “SOCS: physiological suppressors of cytokine signaling” Journal of Cell Science, 2000, 113(Pt 16):2813-9.
Kühl et al., “Comparative analysis of Ebola virus glycoprotein interactions with human and bat cells” J Infect Dis, 2011; 204 Suppl 3:S840-9.
Kwera, “Thinking the unthinkable: Asymptomatic or Airborne Ebola in Uganda?” Scott McPherson's Web Presence, 2007 Dec. 3; <http://scottmcpherson.net/j ournal/2007/12/3/thinking-the-unthinkable-asymptomatic-or-airborne-ebola-in-u.html>
Laborde et al., “Immune complexes inhibit differentiation, maturation, and function of human monocyte-derived dendritic cells” J Immunol, 2007; 179(1):673-81.
Lafon et al., “Evidence for a viral superantigen in humans” Nature, 1992; 358(6386):507-10.
Laminger et al., “Bats and other reservoir hosts of Filoviridae. Danger of epidemic on the African continent?—a deductive literature analysis” Wien Klin Wochenschr, 2010; 122 Suppl 3:19-30.
Lander, “Ebola Unburied Does Improper Burial of Ebola Victims Leave communities at Risk of Exposure Through Dogs” 2014, Aug. 25; http://www.pathogenperspectives.com/2014/08/ebola-unburied-does-improper-burial-of.html
Lauletta et al., “Hepatitis C virus infection and mixed cryoglobulinemia” Clin Dev Immunol, 2012; 2012:502156. Published Online 2012 Jul. 10.
Ledru et al A study of Toxoplasma and cytomegalovirus serology in tuberculosis and in HIV infected patients in Burkina Faso Acta Trop 59 2 149 54 1995
Lee et al Structure of the Ebola virus glycoprotein bound to an antibody from a human survivor NIH Public Access 2008 July 10 454 7201 177 182
Lee et al Pathogenesis of Lassa Fever Virus Infection I Susceptibility of Mice to Recombinant Lassa GP LCMV chimeric Virus Virology 2013 Aug. 1 442 2 114 121
Leroy et al Early immune responses accompanying human asymptomatic Ebola infections Clin Exp Immunol 2001 124 353-460
Leroy et al A Serological Survey of Ebola Virus Infection in Central African Nonhuman Primates JID 2004 1 Dec.; 190(11):1895-9.
Leroy et al Fruit bats as reservoirs of Ebola virus Nature 438 575 576 Dec. 1 2005
Leroy et al Multiple Ebola Virus Transmission Events and Rapid Decline of Central African Wildlife Science 16 Jan. 2004 Vol 303 no 5656 pp 387 390
Leroy et al The Lancet Vol 355 Jun. 24 2000 Human Asymptomatic Ebola infection and strong inflammatory response
Leroy et al Human Ebola outbreak resulting from direct exposure to fruit bats in Luebo, Democratic Republic of Congo, 2007 Vector Borne Zoonotic Dis 2009 Dec. 9 6 723-8
Leung et al Ebolavirus VP35 suppresses IFN production from conventional but not plasmacytoid dendritic cells Immunol Cell Biol 2011 Oct. 89 7 792 802
Levinsky, Role of circulating immune complexes in renal diseases J Clin Pathol 1981 34 1214 1222
Lewis et al Immune Serum Increases Arenavirus Replication in Monocytes J gen Virol 1988 69 1735 1739
Liang et al., Gammaherpesvirus-driven plasma cell differentiation regulates virus reactivation from latently infected B lymphocytes, PLoS Pathogens November 2009; 5 Issue 11 e1000677
Lin et al Different types of cell death induced by enterotoxins Toxins (Basel) 2010 2 2158-76
Linderholm et al Elevated plasma levels of tumor necrosis factor (TNF)-alpha, soluble TNF receptors, interleukin (IL)-6, and IL-10 in patients with hemorrhagic fever with renal syndrome Journal of Infectious Diseases 1996 173 38 43
List of approved mAbs (Monoclonal Antibodies) <http://en.wikipedia.org/wiki/List_of_monoclonal_antibodies>
Liu et al Membranous Glomerulonephropathy Associated with Hepatitis B Virus Infection, HK J Paediatr (New Series) 2008; 13:23-29. <http://www.hkjpaed.org/details.asp?id=643&show=1234>
Liu et al Molecular mimicry of human endothelial cell antigen by autoantibodies to nonstructural protein 1 of dengue virus J Biol Chem Mar. 18 2011 286 11 9726 9736
Liu et al Origin of the human malaria parasite Plasmodium falciparum in gorillas Nature 2010 September 23 467 7314 420 425
Lizarraga et al Dengue-associated kidney disease J Nephropathol 2014 3 2 57 62
Lucas et al ERK activation following macrophage FcgammaR ligation leads to chromatin modifications at the IL-10 locus J Immunol 2005 175 1 469 77
Lund et al Increased adhesion as a mechanism of antibody-dependent and antibody-independent complement-mediated enhancement of human immunodeficiency virus infection Journal of Virology apr 1995 p 2393-400.
Madson et al Cytokine levels in serum and synovial fluid of patients with juvenile rheumatoid arthritis J Rheumatol 1994 21 12 2359 63
Mady et al Antibody-dependent enhancement of dengue virus infection mediated by bispecific antibodies against cell surface molecules other than Fc gamma receptors J Immunol 1991 Nov. 1 147 9 3139 44
Mady et al Neuraminidase augments Fc gamma receptor II-mediated antibody-dependent enhancement of dengue virus infection Journal of General Virology 1993 74 839 844
Maganga et al Is Marburg Virus Enzootic in Gabon? Journal of Infectious Diseases 2011 204 S800 S803
Macrophages is Dependent on Interleukin 6
Mahalingam et al Call to action for dengue vaccine failure Emerg Infect Dis, 2013 Volume 19 Number 8:1335-7.
Mahan et al Platelet involvement in experimental immune complex-mediated glomerulonephritis in the nonhuman primate Kidney Int 1993 44 4 716 25
Mahanty et al., Cutting edge: impairment of dendritic cells and adaptive immunity by Ebola and Lassa viruses J Immunol 2003 170 2797 2801
Maidji et al Maternal antibodies enhance or prevent cytomegalovirus infection in the placenta by neonatal Fc receptor-mediated transcytosis American Journal of Pathology 2006; Vol 168 No 4
Maiga et al Fc gamma receptor IIa-H131R polymorphism and malaria susceptibility in sympatric ethnic groups, Fulani and Dogon of Mali Scand J Immunol, 2014 January; 79(1):43-50.
Maillard et al Fcγ part of IgG J Biol Chem 2004 279 4 2430 7 Fc gamma receptor like activity of hepatitis C virus core protein
Maillard et al Fcgamma receptor-like activity of hepatitis C virus core protein J Biol Chem 2004 279 2430 2437
Malaria.com “Disease Burdens Shifting from Communicable to Chronic Diseases” 2011 March 14; <http://www.malaria.com/news/communicable-chronic-disease-shif>
Malaria No More, “How Ebola Makes Malaria More Deadly” 2014 Aug. 13; <https://www.malarianomore.org/news/blog/how-ebola-makes-malaria-more-deadly>
Maiga et al Human candidate polymorphisms in sympatric ethnic groups differing in malaria susceptibility in Mali, PLoS One October 2013 Vol 8 Issue 10 e75675
Malhotra et al Transcriptional profiling of the circulating immune response to lassa virus in an aerosol model of exposure PLoS Neglected Tropical Diseases April 2013 Vol 7 Issue 4 e2171
Mannoor et al Essential role of extrathymic T cells in protection against malaria J Immunol 2002 169 301 306
Marsh et al Ebola Reston virus infection of pigs: clinical significance and transmission potential Journal of Infectious Diseases 2011 204 S804 S809
Marta et al Proinflammatory cytokines and elastase-alpha-1-antitrypsin in Argentine hemorrhagic fever Am J Trop Med Hyg 60 1 1999 pp 85 89
Martinez et al The role of antigen-presenting cells in filoviral hemorrhagic fever: gaps in current knowledge NIH Antiviral Res 2012 March 93 3 416 428
Martinez, et al. “A Mutation in the Ebola Virus Envelope Glycoprotein Restricts Viral Entry in a Host Species- and Cell-Type Specific Manner” J. Virol, 2013 March; 87(6):3324-34.
Martinez et al Ebola virus exploits a monocyte differentiation program to promote its entry Journal of Virology April 2013 Volume 87 no 7 p 3801 3814
Marzi et al Analysis of the interaction of Ebola virus glycoprotein with DC-SIGN (dendritic cell-specific intercellular adhesion molecule 3-grabbing nonintegrin) and its homologue DC-SIGNR JID 2007 196 Suppl 2 S237
Marzi et al DC-SIGN and DC-SIGNR interact with the glycoprotein of Marburg virus and the S protein of severe acute respiratory syndrome coronavirus, J Virol November 2004 p 12090-5
Max-Planck-Gesellschaft. “West African bats: No safe haven for malaria parasites.” ScienceDaily. ScienceDaily, 21 Oct. 2013. <www.sciencedaily.com/releases/2013/10/131021130745.htm>.
Mayadas et al Mechanisms of immune complex-mediated neutrophil recruitment and tissue injury NIH November 2009 17 120 20 2012 2024
McCormick et alLassa fever. Effective therapy with ribavirin N Engl J Med 1986 Jan. 2 314 1 20 6
McIntosh et al An engineered non-toxic superantigen increases cross presentation of hepatitis B virus nucleocapsids by human dendritic cells PLoS One April 2014 Vol 9 Issue 4 e93598
McLay et al Targeting virulence mechanisms for the prevention and therapy of arenaviral hemorrhagic fever Antiviral Res 2013 February 97 2 81 92
Medecins Sans Frontieres/Doctors without borders, “Liberia: 300,000 people at risk of malaria as Ebola epidemic damages healthcare system” 2014 Oct. 30; <http://www.msf.org.uk/article/liberia-300-000-people-at-risk-of-malaria-as-ebola-epidemic-damages-healthcare-system>
Meltzer et al. “Estimating the Future number of cases in the Ebola Epidemic—Liberia and Sierra Leone, 2014-2015” Morbidity and Mortality Weekly Report Surveill Summ, 2014 Sep. 26; 63 Supple 3:1-14.
Metenou et al Impact of filarial infections on coincident intracellular pathogens: Mycobacterium tuberculosis and Plasmodium falciparum Curr Opin HIV Aids 2012 May 7 3 231 238
Meyer et al Antibody-dependent enhancement of hepatitis C virus infection J Virol 2008 March 82 5 2140 9 Epub 2007 Dec. 19
Meyer et al A weak neutralizing antibody response to hepatitis C virus envelope glycoprotein enhances virus infection PLoS One 2011 6 8 e23699
Mibei et al Immune complex levels in children with severe plasmodium falciparum malaria Am J Trop Med Hyg 72 5 2005 pp 593-9.
Michowitz et al. “The involvement of tumor necrosis factor-related apoptosis-inducing ligand (TRAIL) in atherosclerosis” J Am Coll Cardiol, 2005 April; 45(7):1018-24.
Miethke et al Superantigen mediated shock: a cytokine release syndrome Immunobiology, 1993 189 3 4:270-84.
Miller et al Filovirus entry into cells—new insights Curr Opin Virol 2012 April; 2 2 206 214.
Mimoto et al, Crystal structure of a novel asymmetrically engineered Fc variant with improved affinity for FcγRs Mol Immunol, 2014, March; 58(1):132-8. Published Online 2013, Dec. 14.
Mimoto et al, Engineered antibody Fc variant with selectively enhanced FcγRIIb binding over both FcγRIIa(R131) and FcγRIIa(H131) J Protein Eng Des Sel, 2013 October; 26(10):589-98. Published online 2013, June 5.
Minamishima et al Neutralizing antibodies to cytomegaloviruses in normal simian and human sera Infect Immun, 1971; 4(4):368-73.
Minopetrou et al., Hepatitis C virus (HCV)-related cryoglobulinemia: cryoglobulin type and anti-HCV profile Clin Vaccine Immunol, 2013; 20 5 698
Miranda et al Reston ebolavirus in humans and animals in the Philippines: a review Journal of Infectious Diseases 2011 204 S757 S760
Misasi et al Camouflage and misdirection: the full-on assault of ebola virus disease Cell Oct. 23 2014; 159
Mitchell et al “Inactivation of a common epitope responsible for the induction of antibody-dependent enhancement of HIV” AIDS, 1998 Jan. 22; 12(2):147-56
MMWR CDC Nov. 18 2014 “Update: Ebola Virus Disease Epidemic—West Africa, November 2014” 2014, November 18; Early Release/Vol. 63, 3 pages. <http://www.cdc.gov/mmwr/pdf/wk/mm63e1118.pdf>
Modhiran et al., “Subversion of innate defenses by the interplay between DENV and pre-existing enhancing antibodies: TLRs signaling collapse” PLoS Negl Trop Dis, 4 12 e924.
Moreland, “Cytokine release syndrome” in Rheumatology and Immunology Therapy; A to Z Essentials. Moreland (Ed.) Springer Berlin Heidelberg New York 2004. Pages 268-269.
“Much worse to come: The Ebola epidemic in west Africa poses a catastrophic threat to the region, and could yet spread further” The Economist Oct. 18 2014
Mukherjee et al Plasmodium falciparum-free merozoites and infected RBCs distinctly affect soluble CD40 ligand-mediated maturation of immature monocyte-derived dendritic cells Journal of Lukoc Biol, 2008; 84(1):244-54.

Munakata et al., Human parvovirus B19 infection of monocytic cell line U937 and antibody-dependent enhancement Virology, 2006; 345(1):251-7.
Muro et al., “Defect of Fc receptors and phenotypical changes in sinusoidal endothelial cells in human liver cirrhosis” Am J Pathol, 1993; Vol 143 No 1:105-20.
Murray et al., “Global, regional, and national incidence and mortality for HIV, tuberculosis, and malaria during 1990-2013: a systematic analysis for the Global Burden of Disease Study 2013” 2014 September 13; 384(9947):1005-70. Published Online 2014 Jul. 22.
Mwanatambwe et al., “Ebola hemorrhagic fever (EHF): mechanism of transmission and pathogenicity” J Nippon Med Sch, 2001; 68(5):370-5.
Nagalo et al “Seroprevalence and incidence of transfusion-transmitted infectious diseases among blood donors from regional blood transfusion centres in Burkina Faso, West Africa” Trop Med Int Health, 2012; vol 17 No 2:247-53.
Nagashunmugam et al “In vivo immune evasion mediated by the herpes simplex virus type 1 immunoglobulin G Fc receptor” J Virol, 1998; 72(7):5351-9.
Nardella et al “Fc epitopes for human rheumatoid factors and the relationships of rheumatoid factors to the Fc binding proteins of microorganisms” Scand J Rheumatol Suppl, 1988; 75:190-8.
National Institute of Allergy and Infectious Diseases “Understanding Malaria, Fighting an Ancient Scourge” 2007; NIH Publication No. 07-7139, <http://www.niaid.nih.gov/topics/Malaria/Documents/malaria.pdf>
Ndjamen et al “The herpes virus Fc receptor gE-gI mediates antibody bipolar bridging to clear viral antigens from the cell surface” PLoS Pathogens, 2014 Mar. 6; 10(3):e1003961.
Newman, “Relegating malaria resurgences to history” Malar J, 2012; 11:123.
Nfon et al “Immunopathogenesis of Severe Acute Respiratory Disease in Zaire ebolavirus-infected Pigs” PLoS One, 2013; 8(4):e61904.
Nguyen et al., “Cynomolgus and pigtail macaque IgG subclasses: characterization of IGHG genes and computational analysis of IgG/Fc receptor binding affinity” Immunogenetics, 2014 June; 66(6):361-77.
Niklasson et al “Detection of Lassa virus antigens and Lassa virus-specific immunoglobulins G and M by enzyme-linked immunosorbent assay” J Clin Microbiol, 1984; 20(2):239-44.
Noone et al “Plasma cytokines, chemokines and cellular immune responses in pre-school Nigerian children infected with Plasmodium falciparum” Malar J, 2013; 12:5.
Noris et al “STEC-HUS, atypical HUS and TTP are all diseases of complement activation” Nat Rev Nephrol, 2012; 8(11):622-33.
Normile, “Emerging infectious diseases. Scientists puzzle over Ebola-Reston virus in pigs” Science, 2009; 323(2913):451.
O'Carroll, “Fighting Ebola in Sierra Leone: ‘The world is not safe’” The Guardian, 2014 Oct. 31; <http://www.theguardian.com/world/20 14/oct/30/ebola-sierra-leone-fight-who-disease-fight>
O'Carroll, “New Ebola outbreak in Sierra Leone raises fears of new infection chain” The Guardian, 2014 Nov. 4; <http://www.theguardian.com/world/2014/nov/04/ebola-outbreak-sierra-leone>
O'Flaherty et al., “The murine gammaherpesvirus immediate-early Rta synergizes with IRF4, targeting expression of the viral M1 superantigen to plasma cells” PLos Pathogens August 2014 Vol 10 Issue e1004302.
O'Shea et al “Bat Flight and Zoonotic Viruses” Emerg Infect Dis, 2014 May
Odera et al., Dual role of erythrocyte complement receptor type 1 in immune complex-mediated macrophage stimulation: implications for the pathogenesis of Plasmodium falciparum malaria” Clin Exp Immunol, 2011; 166(2):201-7.
Ogbu et al “Lassa fever in West African sub-region: an overview” J Vector Borne Dis, 2007; 44(1):1-11.
Okamoto et al “Definition of the region on NS3 which contains multiple epitopes recognized by dengue virus serotype-cross-reactive and flavivirus-cross-reactive, HLA-DPw2-restricted CD4+ T cell clones” J Gen Virology, 1998; 79 697 704
Okamoto et al “Preferential usage of T-cell receptor V beta 17 by dengue virus-specific human T lymphocytes in a donor with immunity to dengue virus type 4” Journal of Virology, 1994; 7614-9.
Olival et al., “Ebola Virus Antibodies in Fruit Bats, Bangladesh” Emerg Infect Dis, 2013; 19(2):270-3.
Olival et al. “Filoviruses in Bats: Current Knowledge and Future Directions” Viruses, 2014 Apr. 17; 6(4):1759-88.
Olson et al “Dead or alive: animal sampling during Ebola hemorrhagic fever outbreaks in humans” Emerg Health Threats J, 2012; 5.
Opsenica et al., “A chemotype that inhibits three unrelated pathogenic targets: the botulinum neurotoxin serotype A light chain, P. falciparum malaria, and the Ebola filovirus” J Med Chem 2011 March; 54 5 1157-69.
Osiowy et al “Antibody-dependent enhancement of respiratory syncytial virus infection by sera from young infants” Clinical and Diagnostic Laboratory Immunology, 1994; 1(6):670-7.
Ou et al “Staphylococcus aureus superantigens are associated with chronic rhinosinusitis with nasal polyps: a meta-analysis” Eur Arch Otorhinolaryngol, 2014 October; 271(10):2729-36. Published Online 2014 Mar. 7.
Oyeyinka et al “The role of circulating immune complexes; antinuclear and rheumatoid factor autoantibodies in aging in Nigerians” Mech Ageing Dev, 1995; 85(2-3):73-81.
Paaijmans et al “Understanding the link between malaria risk and climate” PNAS USA, 2009; 106(33):13844-9.
Paessler et al., “Pathogenesis of the viral hemorrhagic fevers” Annu Rev Pathol, 2013; 8:411-40.
Paganelli et al “Circulating immune complexes in onchocerciasis” Clin Exp Immunol, 1980; 39 570 575
Pan et al., “Reston virus in domestic pigs in China” Arch Virol 2014 159 5 1129 32
Panchal et al “Induced IL-10 splice altering approach to antiviral drug discovery” Nucleic Acid Therapeutics, 2014 June; 24(3):179-85. Published Online 2014 Mar. 21.
Pandey et al “Interactive effects of immunoglobulin gamma and human leucocyte antigen genotypes on clearance and persistence of infection with hepatitis C virus” Clin Exp Immunol, 2007; 150(3):518-22.
Pandey et al “Genetic markers of IgG influence the outcome of infection with hepatitis C virus” J infect Dis 2008 198 9 1334-6
Parren et al “Antibodies in human infectious disease” Immunologic Research 2000; 21(2-3):265-78.
Parren et al “Pre- and Postexposure Prophylaxis if Ebola Virus Infection in an Animal Model by Passive Transfer of a Neutralizing Human Antibody” J Virol 2002 76 12 6408-6412
Pascual et al “Malaria resurgence in the East African highlands: temperature trends revisited” PNAS USA, Apr. 11 2006 vol 103 no 15 5829 5834
Patel et al “Specificity of staphylococcal superantigen-like protein 10 toward the human IgG1 Fc domain” J Immunol 2010 184 6283-92.
Peter et al “Attenuated antigen-specific T cell responses in cirrhosis are accompanied by elevated serum interleukin-10 levels and down-regulation of HLA-DR on monocytes” BMC Castroenterology 2013 13:37.
Peters et al “Ebola and Hantaviruses” FEMS Immunol Med Microbiol, 1997; 18(4):281-9.
Peters, “An introduction to Ebola: the virus and the disease” J Infect Dis, 1999; 179 (Supplement 1) ix-xvi.
Pied et al “Evidence for superantigenic activity during murine malaria infection” Intl Immunolgy 1997; 9(1):17-25.
Pigott et al “Funding for malaria control 2006-2010: a comprehensive global assessment” Malaria Journal, 2012; 11:246
“Plagemann et al “Polyclonal hypergammaglobulinemia and formation of hydrophobic immune complexes in porcine reproductive and respiratory syndrome virus-infected and uninfected pigs” Viral Immunol. 2005; 18(1):138-47.
Polimeni et al “Haemozoin induces early cytokine-mediated lysozyme release from human monocytes through p38 MAPK- and NF-kappaB-dependent mechanisms” PLoS ONE, 2012; 7 Issue 6:e39497.
Pourrut et al “Large serological survey showing cocirculation of Ebola and Marburg viruses in Gabonese bat populations, and a high seroprevalence of both viruses in Rousettus aegyptiacus” BMC Infectious Diseases, 2009; 9:159.
Pourrut et al “Spatial and temporal patterns of Zaire ebolavirus antibody prevalence in the possible reservoir bat species” The Journal of Infectious Diseases 2007 196 S176-83.
Qiao et al “Porcine FcγRIIb mediates enhancement of porcine reproductive and respiratory syndrome virus (PRRSV) infection” PloS One, 2011; 6(12):e28721.
Quidel Corporation, “Enzyme immunoassay for the quantitation of circulating immune complexes CIC in human plasma or serum” A5316H 2009 04 <http://www.quidel.com/sites/default/files/product/documents/a001_microvue_cic-c1q_english_0.pdf>
Ramsland et al, “Structural basis for Fc gammaRIIa recognition of human IgG and formation of inflammatory signaling complexes” J Immunology 2011; 187:3208-17.
Rasmussen et al “Host genetic diversity enables Ebola hemorrhagic fever pathogenesis and resistance” Science; 2014 Nov. 21; 346(6212):987-91. Published Online 2014 Oct. 30.
Rodrigo et al., “Dengue virus neutralization is modulated by IgG antibody subclass and Fcgamma receptor subtype” Virology, 2009; 394(2):175-82.
Rodrigo et al “Differential enhancement of dengue virus immune complex infectivity mediated by signaling-competent and signaling-incompetent human Fcgamma RIA (CD64) or FcgammaRIIA (CD32)” J Virol, 2006 80 20 10128-38.
Rogers et al “IgG Fc receptor III homologues in nonhuman primate species: genetic characterization and ligand interactions” J Immunol 2006; 177(6):3848-56.
Rogers “Malaria deaths country by country: how many are there?” The Guardian, 2012 Feb. 3; <http://www.theguardian.com/news/datablog/2012/feb/03/malaria-deaths-mortality>
Rolph et al “Downregulation of interferon-3 in antibody-dependent enhancement of dengue viral infections of human macrophages is dependent on interleukin-6” Journal of Infectious Diseases 2011 204 489-91.
Rotman et al “Fc receptors are not required for antibody-mediated protection against lethal malaria challenge in a mouse model” J immunol, 1998; 161(4):1908-12.
Rovira-Vallbona et al “Low antibodies against Plasmodium falciparum and imbalanced pro-inflammatory cytokines are associated with severe malaria in Mozambican children: a case-control study” Malaria Journal, 2012 11 181
Ruberto et al “Knowledge, attitudes and practices of malaria control among communities from the health district of Forecariah in the Republic of Guinea, West Africa” J Vector Borne Dis, 2014 June; 51(2):119-27.
Rubtsov et al “TLR agonists promote marginal zone B cell activation and facilitate T-dependent IgM responses” J Immunl, 2008; 180(6):3882-8.
Russier et al “Immune Responses and Lassa Virus Infection” Viruses, 2012; 4 2766-85
Ryabchikova et al “An analysis of features of pathogenesis in two animal models of Ebola virus infection” J Infect Dis, 1999; 179 suppl 1:S199-202.
Saadoun et al., “Hepatitis C-associated mixed cryoglobulinaemia: a crossroad between autoimmunity and lymphoproliferation” Rheumatology (Oxford), 2007; 46 1234-42.
Saeidnia et al “Ebola hemorrhagic fever: current outbreak and progress in finding a cure” Daru, 2014 Nov. 12; 22(1):70.
Salomon et al PNAS Jul. 24 2007 Vol 104 No 30 12479 12481 Inhibition of the cytokine response does not protect against lethal H5N1 influenza infection
“Sansomo et al, Rheumatology 2007 46 572 578 Hepatitis C virus infection cryoglobulinaemia and beyond”
Sansonno et al “Non-enveloped HCV core protein as constitutive antigen of cold-precipitable immune complexes in type II mixed cryoglobulinaemia” Clin Exp Immunol, 2003; 133(2):275-82.
Santos et al “The epidemiological dimension of TB/HIV co-infection” Rev Lat Am Enfermagem, 2009; 17(5):683-8.
Saphire et al., “A New Look at Rheumatoid Factor,” Cutting Edge Reports, 2001; from http://www.rheuma21st.com pp. 1-9.
Sayama et al “A seroepidemiologic study of Reston ebolavirus in swine in the Philippines” BMC Veterinary Research, 2012; 8:82
Schaer et al “High diversity of West African bat malaria parasites and a tight link with rodent Plasmodium taxa” PNAS USA, 2013; 110(43):17415-9.
Schiffer et al “B-cell-attracting chemokine CXCL13 as a marker of disease activity and renal involvement in systemic lupus erythematosus (SLE)Nephrol Dial Transplant” 2009; 24 3708-12.
Shedlock et al “Induction of broad cytotoxic T cells by protective DNA vaccination against Marburg and Ebola” Mol Ther, 2013; 21(7):1432-44.
Shedlock et al “Antibody-mediated neutralization of Ebola virus can occur by two distinct mechanisms” Virology, 2010; 5 401 2 228 235
Siberil et al, “Molecular aspects of human FcgammaR interactions with IgG: functional and therapeutic consequences” Immunol Lett, 2006; 106(2):111-8.
Silverman et al., “A B cell superantigen-induced persistent “Hole” in the B-1 repertoire” J Exp Med, 2000; 192(1):87-98.
Sino Biological Cynomolgus DC-SIGN/CD209 Gene/cDNA Clone
Sino Biological Inc DC-SIGN/CD209 PROTEIN ANTIBODY ELISA kit cDNA CLONE Slenczka et al “Forty years of Marburg Virus” J Infect Dis, 2007; 196 Suppl 2:S131-5.
Smith et al., “A study of the presence of circulating immune complexes in schistosomiasis” Trans R Soc Trop Med Hyg, 1977; 71 4:343-8.
Sondermann et al “General mechanism for modulating immunoglobulin effector function” PNAS USA, 2013; 110(24):9868-72.
Sprague et al “Crystal structure of the HSV-1 Fc receptor bound to Fc reveals a mechanism for antibody bipolar bridging” PLoS Biol, 2006 4 6 e148
Sprague et al “The human cytomegalovirus Fc receptor gp68 binds the Fc CH2-CH3 interface of immunoglobulin G” J Virol, 2008; 82(7):3490-9.
Stanisic et al “Immunoglobulin G subclass-specific responses against Plasmodium falciparum merozoite antigens are associated with control of parasitemia and protection from symptomatic illness” Infect Immun, 2009; 77(3):1165-75.
Stanojevic et al “Antigen/antibody content of circulating immune complexes in HIV-infected patients” Biomed Pharmacother, 1996 50 10 448 93
Stauch et al “Targeting of natural killer cells by rabbit antithymocyte globulin and campath-1H: similar effects independent of specificity” PLoS One, 2009; Vol 4 Issue 3 e4709
Stewart et al “An Empirical Study of the Evolution of virulence Under Both Horizontal and Vertical Transmission Evolution” 2005; 59(4):730-9
Stillwaggon, Aids and the Ecology of Poverty New York Oxford University Press 2006 Cover page, title page and table of contents.
Strong et al “Stimulation of Ebola virus production from persistent infection through activation of the Ras/MAPK pathway” PNAS USA Nov. 18 2008 vol 105 no 46 17982 17987
Stuckler, “Population causes and consequences of leading chronic diseases: a comparative analysis of prevailing explanations” The Milbank Quarterly, 86 No 2 2008 pp 273 326
Sun et al “Immune complexes activate human endothelium involving the cell-signaling HMGB 1-RAGE axis in the pathogenesis of lupus vasculitis” Laboratory Invest 2013 93 626-38
Syam et al, “Differential recruitment of activating and inhibitory Fc gamma RII during phagocytosis” J Immunology 2010; 184(6):2966-73; Published Online 12 Feb. 2010.
Takada, “Filovirus tropism: cellular molecules for viral entry” Front Microbiol, 2012; 3:34.
Takada et al., “Infectivity-enhancing antibodies to Ebola virus glycoprotein” J Virol, 2001; 75(5):2324-30.
Takano et al “Antibody-dependent enhancement occurs upon re-infection with the identical serotype virus in feline infectious peritonitis virus infection” J Vet Med Sci, 2008; 70(12):1315-21.
Takeda et al., “Two receptors are required for antibody-dependent enhancement of human immunodeficiency virus type 1 infection: CD4 and Fc gamma R” J Virol, 1990; 64(11):5605-10.
Takeda et al “Antibody-enhanced infection by HIV-1 via Fc receptor-mediated entry” Science, 1988; 242(4878):580-3.
Tandler, “Cytomegalovirus in the principal submandibular gland of the little brown bat, Myotis lucifugus” J Comp Pathol, 1996; 114(1):1-9.
Taniguchi et al “Reston Ebolavirus antibodies in bats, the Philippines” Emerg Infect Dis 2011; 17(8):1559-60.
Taylor et al “Seasonal patterns of habitat use by insectivorous bats in a subtropical African agro ecosystem dominated by macadamia orchards” 2013 January; African J of Ecol, <http://www.researchgate.net/profile/Peter_Taylor6/publication/23 6209830_Seasonal_patterns_of_habitat use by insectivorous_bats in_a_subtropical_African_agro-ecosystem_dominated by_macadamia_orchards/links/00463516fd0986e4b1000000.pdf.>
Taylor et al “Filoviruses are ancient and integrated into mammalian genomes” BMC Evolutionary Biology 2010 10 193
Thagia et al “Intestinal epithelial suppressor of cytokine signaling 3 enhances microbial-induced inflammatory tumor necrosis factor-α, contributing to epithelial barrier dysfunction” Am J Physil Gastrointest Liver Physiol 2014 Nov. 6
The Washington Post, “Update: The Ebola epidemic,” available on the world wide web at washingtonpost.com/national/health-science/update-the-ebola-epidemic/2014/10/25/98342c90-5ca7-11 e4-8264-deed989ae9a2_graphic.html
Tisoncik et al. “Into the eye of the cytokine storm” MMB Reviews 2012 March; 76(1): 16-32
Towner et al., “Marburg virus infection detected in a common African bat” PloS One, 2007; 2(8):e764.
Towner et al “Isolation of genetically diverse Marburg viruses from Egyptian fruit bats” PLoS Pathogens July 2009 Vol 5 Issue 7:e1000536
Trist et al “Polymorphisms and interspecies differences of the activating and inhibitory FcγRII of Macaca nemestrina influence the binding of human IgG subclasses” J Immunol 2014 Jan. 15 192 2 792-803
Tsai et al “An emerging role for the anti-inflammatory cytokine interleukin-10 in dengue virus infection” Journal of Biomedical Science 2013 20 40
Tsitsikov et al “Cross-linking of Fc gamma receptors activates HIV-1 long terminal repeat-driven transcription in human monocytes” Int Immunol 1995 10 1665 70
Tsuda et al “A replicating cytomegalovirus-based vaccine encoding a single Ebola virus nucleoprotein CTL epitope confers protection against Ebola virus” Plos Negl Trop Dis August 2011 5 Issue 8 e1275
U S Dept. of Health and Human Services et al. “Understanding Malaria: Fighting an Ancient Scourge” NIH Publication No. 07 7139, 2007 February; <https://www.niaid.nih.gov/topics/Malaria/Documents/malaria.pdf>
Ubol et al., “How Innate Immune Mechanisms Contribute to AntibodyOEnhanced viral Infections” Clin Vaccine Immunol, 2010; 17 12 1829-35.
Ubol et al “Mechanisms of immune evasion induced by a complex of dengue virus and preexisting enhancing antibodies” J. Infect Dic. 2010 Mar. 15; 201(6):923-35.
Urra et al. “Cytokines and Systemic Lupus Erythematosus,” in Systemic Lupus Erythematosus, InTech (2012), Available on the world wide web at intechopen.com/books/systemic-lupus-erythematosus/cytokines-and-systemic-lupus-erythematosus
van der Poel et al “Functional characteristics of the high affinity IgG receptor, FcγRI.J” Immunol 2011 186 2699-2704
Veri et al “Monoclonal antibodies capable of discriminating the human inhibitory Fcgamma-receptor IIB (CD32B) from the activating Fcgamma-receptor IIA (CD32A): biochemical, biological and functional characterization” Immunology 121 293 404
Vidarsson et al “IgG subclasses and allotypes: from structure to effector functions” Frontiers in Immunology October 2014 Vol 5 Article 520
Villar et al “Tumor necrosis factor-α regulates glucocorticoid synthesis in the adrenal glands of Trypanosoma cruzi acutely-infected mice. the role of TNF-R1” PLoS One 8 5 e63814
Visvanathan et al “Inhibition of Bacterial superantigens by Peptides and Antibodies” Infection and Immunity February 2001 p 875 884
Waggoner et al “HCV core protein interaction with gC1q receptor inhibits Th1 differentiation of CD4+ T cells via suppression of dendritic cell IL-12 production” J Leukoc Biol 82 1407 1419 2007
Wahala et al “The human antibody response to dengue virus infection” Viruses 2011; 3(12): 2374-95.
Walt “The Fight Against Ebola Could Lead to Surge in Measles and Malaria” Time Magazine Nov. 11, 2014 <http://time.com/3578870/ebola-measles-malaria/#3578870/ebola-measles-malaria/>
Wan et al “Impairment of the antibody-dependent phagocytic function of PMNs through regulation of the FcγRs expression after porcine reproductive and respiratory syndrome virus infection” PLoS one June 2013 Vol 8 Issue 6 e66965
Wand et al “Antibody-dependent SARS coronavirus infection is mediated by antibodies against spike proteins” Biochem Biophys Res Commun 2014 451 2 208 14
Wang et al “DC-SIGN: binding receptors for hepatitis C virus” Chinese Medical Journal 2004 117 9 1395-400
Warncke et al “Different adaptations of IgG effector function in human and nonhuman primates and implications for therapeutic antibody treatment” J Immunol. 2012 188(9) 4405-11
Wauquier et al “Human fatal zaire ebola virus infection is associated with an aberrant innate immunity and with massive lymphocyte apoptosis” PLoS October 2010 Vol 4 Issue 10 e837
Weingartl et al “Transmission of Ebola virus from pigs to non-human primates” Science Reports 2811
West et al “Clinical Presentation and Management of Severe Ebola Virus Disease” Ann Am Thorac Soc. 2014, November; 11(9):1341-50.
White et al “Molecular architectures of trimeric SIV and HIV-1 envelope glycoproteins on intact viruses: strain-dependent variation in quaternary structure” PLoS Pathogens 2010 6 12 e1001249
Willey et al “Extensive complement-dependent enhancement of HIV-1 by autologous non-neutralising antibodies at early stages of infection” Retrovirology 2011 8 16
Wing et al “Mechanism of first-dose cytokine-release syndrome by CAMPATH 1-H: involvement of CD16 (FcgammaRIII) and CD11a/CD18 (LFA-1) on NK cells” J Clin Invest Vol 98 No 12 Dec. 1996 2819 2826
Wittmann et al “Isolates of Zaire ebolavirus from wild apes reveal genetic lineage and recombinants” PNAS Oct. 23 2007 vol 104 no 43 17123 17127
World Health Organization, “Climate change and health” Media center Fact sheet No. 266 Reviewed August 2014 <http://www.who.int/mediacentre/factsheets/fs266/en/>
World Health Organization, “WHO Global Malaria Programme: World Malaria Report 2012” 2012; <http://www.who.int/malaria/publications/world_malaria_report_2012/wmr2012_full_report.pdf>
World Health Organization Ebola Response Team “Ebola virus disease in West Africa—the first 9 months of the epidemic and forward projections” N Engl J Med, 2014 Oct. 16; 371(16):1481-95.
Wu et al “Strain-specific innate immune signaling pathways determine malaria parasitemia dynamics and host mortality” PNAS Jan. 13 2014 E511 E520
Xiao et al “C1q-bearing immune complexes induce IL-8 secretion in human umbilical vein endothelial cells (HUVEC) through protein tyrosine kinase- and mitogen-activated protein kinase-dependent mechanisms: evidence that the 126 kD phagocytic C1q receptor mediates immune complex activation of HUVEC” Clin Exp Immunol 2001 125 360 367
Xiao et al “Plasmodium falciparum antigen-induced human immunodeficiency virus type 1 replication is mediated through induction of tumor necrosis factor-alpha” J Infect Dis 1998 177 2 437 45
Xiao et al “Understanding PRRSV infection in porcine lung based on genome-wide transcriptome response identified by deep sequencing” PLoS One 2010 Jun. 29 5 6 el 1377
Yaddanapudi et al “Implication of a retrovirus-like glycoprotein peptide in the immunopathogenesis of Ebola and Marburg viruses” FASEB Journal, 2006; 20(14):2519-30.
Yang “China approves Ebola drug for emergency use” Bi World Today, Sep. 5 2014 <http://www.bioworld.com/content/china-approves-ebola-drug-emergency-use-0>
Yang et al “ERK MAP kinase links cytokine signals to activation of latent HIV-1 infection by stimulating a cooperative interaction of AP-1 and NF-kappaB” J Bio Chem 1999; 274(39):27981-8.
Yao et al “Antibody-dependent enhancement of hantavirus infection in macrophage cell lines” Arch Virol 1992 122 1 2 107 18
Yi et al “Hantaan virus RNA load in patients having hemorrhagic fever with renal syndrome: correlation with disease severity” JID 2013 207 1 May 1457-61
Yin et al “Differentially Expressed Genes of Human Microvascular Endothelial Cells in Response to Anti Dengue Virus NS1 Antibodies by Suppression Subtractive Hybridization” Viral Immunology Vol 26 No 3 2013
Yoneto et al “A critical role of Fc receptor-mediated antibody-dependent phagocytosis in the host resistance to blood-stage Plasmodium berghei XAT infection” J Immunol 2001 166 6236 6241
Yoon et al “Antibody-dependent enhancement (ADE) of porcine reproductive and respiratory syndrome virus (PRRSV) infection in pigs” Viral Immunol, 1996; 9(1):51-63.
Yuan et al “Serological evidence of ebolavirus infection in bats, China” Virol J, 2012; 9:236
Yun et al “Pathogenesis of Lassa Fever” Viruses, 2012; 4(10):2031-48
Zekri et al “Characterization of chronic HCV infection-induced apoptosis” Comparative Hepatology 2011 10(1):4.
Zellweger et al “Enhanced infection of liver sinusoidal endothelial cells in a mouse model of antibody-induced severe dengue disease” Cell Hose Microbe 2010 Feb. 18 7 2 128 139
Zhang “Regulatory functions of innate like B cells” Cellular & Molecular Immunology 2013 10
Zhang et al “Dynamic and transient remodeling of the macrophage IL-10 promoter during transcription” J Immunol 2006 July 15 177 2 1282 1288
Zhang et al “Ligation of Fc gamma receptor IIB enhances levels of antiviral cytokine in response to PRRSV infection in vitro” Vet Microbiol 2012 Dec. 7 160 3 4 473 80
Zhou et al “Induction of auto-anti-idiotypic antibodies specific for antibodies to matrix and envelope glycoprotein from pigs experimentally infected with porcine reproductive and respiratory syndrome virus” Vet Immunol Immunopathol 2004 September 101 1 2 49 59
Zigment-Reed et al “Cross-reaction of anti-simian immunodeficiency virus envelope protein antibodies with human immunoglobulins” Scand J Immunol, 2003; 57(3):239-45.

### Other References

Abdul-Majid et al., “Fc receptors are critical for autoimmune inflammatory damage to the central nervous system in experimental autoimmune encephalomyelitis,” Scand. J. Immunol., 2002, 55:70-81.
Abeliovich et al., “Mice lacking α-synuclein display functional deficits in the nigrostriatal dopamine system,” Neuron, 2000, 25:239-252.
Agnello et al., “C1q precipitins in the sera of patients with systemic lupus erythematosus and other hypocomplementemic states: Characterization of high and low molecular weight types,” J. Exp. Med., 1971, 134(3):228-241.
Agnello et al., “Precipitin reactions of the C1q component of complement with aggregated γ-globulin and immune complexes in gel diffusion,” Immunol., 1970, 19:909-919.
Alexianu et al., “Immune reactivity in a mouse model of familial ALS correlates with disease progression,” Neurol., 2001, 57:1282-1289.
Alim et al., “Demonstration of a role for α-synuclein as a functional microtubule-associated protein,” J. Alzheimer's Dis., 2004, 6:435-442.
Anderson et al. “Anti-GPIIb/IIIa (CD41) monoclonal antibodyinduced platelet activation requires Fc receptor-dependent cell-cell interaction,” Br. J. Haematol., 1991, 79:75-83.
Anderson et al., “The Fc receptor for immunoglobulin G (Fc gamma RII) on human platelets,” Semin. Thromb. Hemost., 1995, 21:1-9.
Appel et al., “Immunoglobulins from animals models of motor neuron disease and from human amyotrophic lateral sclerosis patients passively transfer physiological abnormalities to the neuromuscular junction,” Proc. Natl. Acad. Sci. USA, 1991, 88:647-651.
Artandi et al., “Monoclonal IgM rheumatoid factors bind IgG at a discontinuous epitope comprised of amino acid loops from heavy-chain constant-region domains 2 and 3,” Proc. Natl. Acad. Sci. USA, 1992, 89:94-98.
Asbakk et al., “An antigenic determinant is shared by psoriasis-associated p27 antigen and the Fc part of human IgG,” APMIS, 1991, 99:551-556.
Balestrieri et al., “Inhibitory effect of IgM rheumatoid factor on immune complex solubilization capacity and inhibition of immune precipitation,” Arthritis Rheum., 1984, 27(10):1130-1136.
Banci et al., “Fully metallated S134N Cu,Zn-superoxide dismutase displays abnormal mobility and intermolecular contacts in solution,” J. Biol. Chem., 2005, 280(43):35815-35821.
Banci et al., “Human SOD1 before harboring the catalytic metal. solution structure of copper-depleted, disulfide-reduced form” J. Biol. Chem., 2006, 281(4):2333-2337.
Banci et al., “The solution structure of reduced dimeric copper zinc superoxide dismutase. The structural effects of dimerization,” Eur. J. Biochem., 2002, 269:1905-1915.
Betz et al., “De novo design of native proteins: characterization of proteins intended to fold into antiparallel, Rop-like, four-helix bundles,” Biochem., 1997, 36:2450-2458.
Blanchette et al., “Management of chronic immune thrombocytopenic purpura in children and adults,” Semin. Hematol., 1998, 35:36-51.
Blom et al., “Fcγr expression on macrophages is related to severity and chronicity of synovial inflammation and cartilage destruction during experimental immune-complex-mediated arthritis (ICA),” Arthritis Res., 2000, 2:489-503.
Bonelli et al., “Solid phase synthesis of retro-inverso peptide analogues,” Int. J. Peptide Protein Res., 1984, 24:553-556.
Boren and Gershwin, “Inflamm-aging: autoimmunity, and the immune-risk phenotype,” Autoimmunity Rev., 3:401-406, 2004.
Bouras et al., “Humoral immunity in brain aging and Alzheimer's disease,” Brain Research Reviews, 2005, 48:477-487.
Bouras et al., “Induction of MC-I immunoreactivity in axons after injection of the Fc fragment of human immunoglobulins in macaque monkeys,” Acta Neuropathol., 2003, 105:58-64.
Bruijn et al., “Aggregation and motor neuron toxicity of an ALS-Linked SOD1 mutant independent from wild-type SOD1,” Science, 1998, 281:1851-1854.
Bruijn et al., “ALS-linked SOD1 mutant G85R mediates damage to astrocytes and promotes rapidly progressive disease with SOD 1-containing inclusions,” Neuron, 1997, 18:327-338.
Bruijn et al., “Unraveling the mechanisms involved in motor neuron degeneration in ALS,” Annu. Rev. Neurosci., 2004, 27:723-749.
Cassel et al., “Differential expression of FcRIIA, FcRIIB, and FcRIIC in hematopoietic cells: analysis of transcripts,” Mol. Immunol., 1993, 30:451-460.
Check, “Nerve inflammation halts trial for Alzheimer's drug,” Nature, 2002, 415:462.
Chen et al., “Experimental destruction of substantia nigra initiated by Parkinson disease immunoglobulins,” Arch. Neurol., 1998, 55:1075-1080.
Cleveland, “From charcot to SOD1: mechanisms of selective motor neuron death in ALS,” Neuron, 1999, 24:515-520.
Clot et al., “Immunological aspects of psoriasis. III Fc-γ-receptor bearing mononuclear cells in peripheral blood,” Brit. J. Derm., 1978, 99:25-30.
Clynes et al., “Fc receptors are required in passive and active immunity to melanoma,” Proc. Natl. Acad. Sci. USA, 1998, 95:652-656.
Clynes et al., “Inhibitory Fc receptors modulate in vivo cytoxicity against tumor targets,” Nat. Med., 2000, 6(4):443-446.
Clynes et al., “Uncoupling of immune complex formation and kidney damage in autoimmune glomerulonephritis,” Science, 1998, 279:1052-1054.
Clynes and Ravetch, “Cytotoxic antibodies trigger inflammation through Fc receptors,” Immunity, 1995, 3:21-26.
Clynes, “Immune complexes as therapy for autoimmunity,” J Clin Invest., 2005, 115(1):25-27.
Cochran, “Antagonists of protein-protein interactions,” Chemistry & Biology, 2000, 7:R85-R94.
Cochran, “Protein-protein interfaces: mimics and inhibitors,” Curr. Opin. Chem. Biol, 2001, 5:654-659.
Corper et al, “Structure of a human IgM rheumatoid factor Fab bound to its autoantigen IgG Fc reveals a novel topology of antibody-antigen interaction,” Nat Struct Biol., 1997, 4:374-381.
Costa et al., “Non-specific binding of heat-aggregated IgG to histone detected by ELISA,” J. Immunol. Meth., 1984, 74:283-291.
Couillard-Despres et al., “Protective effect of neurofilament heavy chain overexpression in motor neuron disease induced by mutant superoxide dismutase,” Proc. Natl. Acad. Sci. USA, 1998, 95:9626-9630.
Coxon et al., “FcγRIII mediates neutrophil recruitment to immune complexes: a mechanism for neutrophil accumulation in immune-mediated inflammation,” Immunity, 2001, 14:693-704.
Coyle and Procyk-Dougherty, “Multiple sclerosis immune complexes: an analysis of component antigens and antibodies,” Ann. Neurol., 1984, 16:660-667.
Crow et al., “Superoxide dismutase catalyzes nitration of tyrosines by peroxynitrate in the rod and head domains of neurofilament-L,” J. Neurochem., 1997, 69:1945-1953.
Dalaker et al., “Expression of the psoriasis-associated antigen, Pso p27, is inhibited by cyclosporin A,” Acta Derm. Venereol., 1999, 79:281-284.
Das et al., “Amyloid-3 immunization effectively reduces amyloid deposition in FcRγ−/− knock-out mice,” J. Neurosci., 2003, 23(24):8532-8538.
Deckmyn and De Reys, “Functional effects of human antiplatelet antibodies,” Sem Thromb. Hemost., 1995, 21:46-59.
DeLano et al., “Convergent solutions to binding at a protein-protein interface,” Science, 2000, 287:1279-1283.
Demestre et al., “ALS-IgG-induced selective motor neurone apoptosis in rat mixed primary spinal cord cultures,” J. Neurochem., 2005, 94:268-275.
Di Noto et al., “Proteasomal degradation of mutant superoxide dismutases linked to amyotrophic lateral sclerosis,” J. Biol. Chem., 2005, 280(48):39907-39913.
DiDonato et al., “ALS mutants of human superoxide dismutase form fibrous aggregates via framework destabilization,” J. Mol. Biol., 2003, 332:601-615.
Dodel et al., “Intravenous immunoglobulins containing antibodies against beta-amyloid for the treatment of Alzheimer's disease,” J Neurol Neurosurg Psychiatry., 75(10):1472-1474, 2004.
Doucette et al., “Dissociation of human copper-zinc superoxide dismutase dimers using chaotrope and reductant,” J. Biol. Chem., 2004, 279(52):54558-54566.
Duchen, “Contributions of mitochondria to animal physiology: from homeostatic sensor to calcium signaling and cell death,” J. Physiol., 1999, 516:1-17.
Durand et al., “Early abnormalities in transgenic mouse models of amyotrophic lateral sclerosis,” J. Physiol., 2006, 99:211-220.
Easterbrook-Smith et al., “The role of Fc:Fc interactions in insoluble immune complex formation and complement activation,” Mol. Immunol., 1988, 25(12):1331-1337.
Elam et al., “An alternative mechanism of bicarbonate-mediated peroxidation by copper-zinc superoxide dismutase,” J. Biol. Chem., 2003, 278(23):21032-21039.
Elmgreen et al., “Demonstration of circulating immune complexes by the indirect leucocyte phagocytosis test in chronic inflammatory bowel disease,” Acta Med. Scan., 1985, 218:73-78.
Engelhardt et al., “Altered calcium homeostasis and ultrastructure in motoneurons of mice caused by passively transferred anti-motoneuronal IgG,” J. Neuropathol. Exp. Neurol., 1997, 56(1):21-39.
Engelhardt et al., “Stereotaxic injection of IgG from patients with Alzheimer disease initiates injury of cholinergic neurons of the basal forebrain,” Arch. Neurol., 2000, 57:681-686.
Engelhardt et al., “Subcellular localization of IgG from the sera of ALS patients in the nervous system,” Acta Neurol. Scand., 2005, 112:126-133.
Ezaki et al., “Human monoclonal rheumatoid factors augment arthritis in mice by the activation of T cells,” Clin. Exp. Immunol., 1996, 104:474-482.
Ferraroni et al., “The crystal structure of the monomeric human SOD mutant F50E/G51E/E133Q at atomic resolution. The enzyme mechanism revisited,” J. Mol. Biol., 1999, 285:413-426.
Ferri et al., “Cell death in amyotrophic lateral sclerosis: interplay between neuronal and glial cells,” FASEB J., 2004, 18(11):1261-1263.
Fossati et al., Fcγ receptors in autoimmune diseases: Eur. J. Clin. Invest., 2001, 31:821-831.
Frangione and Milstein, “Variations in the S-S bridges of immunoglobins G: interchain disulphide bridges of γG3 myeloma proteins,” J. Mol. Biol., 1968, 33:893-906.
Fratantoni et al., “Uptake of immunoglobulin G from amyotrophic lateral sclerosis patients by motor nerve terminals in mice,” J. Neurol. Sci., 1996, 137:97-102.
Fujiwara et al., “Different immunoreactivity against monoclonal antibodies between wild-type and mutant copper/zinc superoxide dismutase linked to amyotrophic lateral sclerosis,” J. Biol. Chem., 2005, 280(6):5061-5070.
Furukawa and O'Halloran, “Amyotrophic lateral sclerosis mutations have the greatest destabilizing effect on the apo- and reduced form of SOD1, leading to unfolding and oxidative aggregation,” J. Biol. Chem., 2005, 280(17): 17266-17274.
Garcia et al., “Mutations in neurofilament genes are not a significant primary cause of non-SOD 1-mediated amyotrophic lateral sclerosis,” Neurobiol. Dis., 2006, 21:102-109.
Gergely and Sarmay, “Fcγ receptors in malignancies: friends or enemies?” Adv. Cancer Res., 1994, 64:211-245.
Ghetie and Ward, “Multiple roles for the major histocompatibility complex class I-related receptor FcRn,” Ann. Rev. Immunol., 2000, 18:739-766.
Girkontaite et al., “Immunochemical study of human immunoglobulin G Fc region,” Cancer Biother. Radiopharm., 1996, 11:87-96.
Glader et al., “The proatherogenic properties of lipoprotein(a) may be enhanced through the formation of circulating immune complexes containing Chlamydia pneumoniae—specific IgG antibodies,” Eur. Heart J., 2000, 21:639-646.
Gomez-Guerrero et al., “Administeration of IgG Fc fragments prevents glomerular injury in experimental immune complex nephritis,” J. Immunol., 2000, 164:2092-2101.
Goss et al., “Bicarbonate enhances the peroxidase activity of Cu,Zn-SOD,” J Biol Chem., 1999, 274:28233-28239.
Guddat et al., “Local and Transmitted conformational changes on complexation of an anti-sweetener Fab,” J. Mol. Biol., 1994, 236:247-274.
Gussin et al., “Effect of circulating immune complexes on the binding of rheumatoid factor to histones,” Ann. Rheum. Dis., 2000, 59:351-358.
Gussin et al., “Noncognate binding to histones of IgG from patients with idiopathic systemic lupus erythematosus,” Clin. Immunol., 2000, 96(2):150-161.
Haake et al, “The Modificiaton of human immunoglobulin binding to staphylococcal protein A using diethylpyrocarbonate,” J Immunol., 1982, 129:190.
Hall et al., “Relationship of microglial and astrocytic activation to disease onset and progression in a transgenic model of familial ALS,” GLIA, 1998, 23:249-256.
Hamano et al., “Immune complex and Fc Receptor-mediated augmentation of antigen presentation for in vivo Th cell responses,” J. Immunol., 2000, 164:6113-6119.
Harrington et al., “Demonstration of a thrombocytopenic factor in the blood of patients with thrombocytopenic purpura,” J. Lab. Clin. Med., 1951, 38:1-10.
Harris et al., “Refined structure of an intact IgG2a monoclonal antibody,” Biochemistry, 1997, 36:1581-1597.
Hayward et al., “Decreased metallation and activity in subsets of mutant superoxide dismutases associated with familial amyotrophic lateral sclerosis,” J. Biol. Chem., 2002, 277(18):15923-15931.
He et al., “Role of Fcγ receptors in nigral cell injury induced by Parkinson disease immunoglobulin injection into mouse substantia nigra,” Exp. Neurol., 2002, 176:322-327.
Henkel et al., “Presence of dendritic cells, MCP-1, and activated microglia/macrophages in amyotrophic lateral sclerosis spinal cord tissue,” Ann. Neurol., 2004, 55:221-235.
Henkel et al., “The chemokine MCP-1 and the dendritic and myeloid cells it attracts are increased in the mSOD1 mouse model of ALS,” Mol. Cell. Neurosci., 2006, 31(3):427-437.
Hilbush et al., “New prospects and strategies for drug target discovery in neurodegenerative disorders,” NeuroRx, 2005, 2:627-637.
Hock et al., “Generation of antibodies for beta-amyloid by vaccination of patients with AD,” 2002, Nat Med., 8:1270-1275.
Holmdahl et al., “Generation of monoclonal rheumatoid factors after immunization with collagen II-anti-collagen II immune complexes. An anti-idiotypic antibody to anti-collagen II is also a rheumatoid factor,” Scand. J. Immunol., 1986, 24:197-203.
Hoover et al., “Modulation of growth and differentiation of murine myeloma cells by immunoglobulin binding factors,” Curr. Top. Microbiol. Immunol., 1990, 166:77-85.
Hora et al., “Receptors for IgG complexes activate synthesis of monocyte chemoattractant peptide 1 and colony-stimulating factor 1,” Proc. Natl. Acad. Sci. USA, 1992, 89:1745-1749.
Hough et al., “Dimer destabilization in superoxide dismutase may result in disease-causing properties: Structures of motor neuron disease mutants,” Proc. Natl. Acad. Sci. USA, 2004, 101(16):5976-5981.
Hyun et al., “Proteasomal inhibition causes the formation of protein aggregates containing a wide range of proteins, including nitrated proteins,” J Neurochem., 2003, 363-373.
Iivanainen, “The significance of abnormal immune responses in patients with multiple sclerosis,” J. Neuroimmunol., 1981, 1:141-172.
Indik et al., “Human Fc gamma RII, in the absence of other Fc gamma receptors, mediates a phagocytic signal,” J. Clin. Invest., 1991, 88:1766-1771.
Jaarsma et al., “Human Cu/Zn superoxide dismutase (SOD1) overexpression in mice causes mitochondrial vacuolization, axonal degeneration, and premature motoneuron death and accelerates motoneuron disease in mice expressing a familial amyotrophic lateral sclerosis mutant SOD1,” Neurobiology of Disease, 2000, 7:623-643.
Jackson, “Contributions of protein structure-based drug design to cancer chemotherapy,” Seminars in Oncology, 1997, 24(2):164-172.
Jefferis et al., “Immunogenic and antigenic epitopes of immunoglobulins. VIII. A human monoclonal rheumatoid factor having specificity for a discontinuous epitope determined by histidine/arginine interchange as residue 435 of immunoglobulin G,” Immunol. Lett., 1984, 7:191-194.
Jones et al., “Structure-based design of lipophilic quinazoline inhibitors of thymidylate synthase,” J. Med. Chem., 1996, 39:904-917.
Jonsson et al., “Minute quantities of misfolded mutant superoxide dismutase-1 cause amyotrophic lateral sclerosis,” Brain, 2004, 127:73-88.
Kabat et al., Sequences of Proteins of Immunological Interest, 5th Edition, Public Health Service, U.S. Department of Health and Human Services, Bethesda, Md., [Table of Contents] 11 pages, 1991.
Kabat et al., Sequences of Proteins of Immunological Interest, Fourth Edition, 1987, U.S. Department of Health and Human Services, Public Health Service National Institutes of Health, [Table of Contents] 4 pages, 1987.
Khare et al., “The rate and equilibrium constants for a multistep reaction sequence for the aggregation of superoxide dismutase in amyotrophic lateral sclerosis,” Proc. Natl. Acad. Sci. USA, 2004, 101(42):15094-15099.
Kim et al., “Oxidative modification of neurofilament-L by the Cu,Zn-superoxide dismutase and hydrogen peroxide system,” Biochimie, 2004, 86:553-559.
Kitazawa et al., “Microglia as a potential bridge between the amyloid b-peptide and tau,” Ann NY Acad. Sci., 2004, 1035:85-103.
Kleinau et al., “Induction and Suppression of collagen-induced arthritis is dependent on distinct Fcγ receptors,” J. Exp. Med., 2000, 191(9):1611-1616.
Kong and Xu, “Massive mitochondrial degeneration in motor neurons triggers the onset of amyotrophic lateral sclerosis in mice expressing a mutant SOD1,” J. Neurosci., 1998, 18(9):3241-3250.
Koroleva et al., “Binding of complement subcomponent C1q to Streptococcus pyogenes: evidence for interactions with the M5 and FcRA76 proteins,” FEMS Immunol. Med. Microbiol., 1998, 20:11-20.
Kotz et al., “Phage-display as a tool for quantifying protein stability determinants,” Eur. J. Biochem., 2004, 271:1623-1629.
Kowall et al., “In vivo neurotoxicity of beta-amyloid [β(1-40)] and the β(25-35) fragment,” Neurobiol of Aging, 1992, 13:537-542.
Kuo et al., “Hyperexcitability of cultured spinal motoneurons from presymptomatic ALS mice,” J. Neurophysiol., 2004, 91:571-575.
Kuruvilla et al., “Dengue virus infection and immune response in humanized RAG2^{-/-} γc^{-/-} (RAG-hu) mice,” Virol., 2007, 369:143-152.
Landsbury Jr., “Back to the future: the ‘old-fashioned’ way to new medications for neurodegeneration,” Nat. Rev Neurosci., 2004, S51-S57.
Le et al., “Microglial activation and dopaminergic cell injury: an in vitro model relevant to Parkinson's disease,” J. Neurosci., 2001, 21(21):8447-8455.
Leach et al., “Isolation from human placenta of the IgG transporter, FcRn, and localization to the syncytiotrophoblast,” J. Immunol., 1996, 157:3317-3322.
Levy et al., “Protein topology determines binding mechanism,” Proc. Natl. Acad. Sci. USA, 2004, 101(2):511-516.
Lin et al., “3′ Untranslated region in a light neurofilament (NF-L) mRNA triggers aggregation of NF-L and mutant superoxide dismutase 1 proteins in neuronal cells,” J. Neurosci., 2004, 24(11):2716-2726.
Lindberg et al., “Folding of human superoxide dimutase: Disulfide reduction prevents dimerization and produces marginally stable monomers,” Proc. Natl. Acad. Sci. USA, 2004, 101(45):15893-15898.
Lindberg et al., “Systematically perturbed folding patterns of amyotrophic lateral sclerosis (ALS)-associated SOD1 mutants,” Proc. Natl. Acad. Sci. USA, 2005, 102(28):9754-9759.
Liu et al., “β2-microglobulin-deficient mice are resistant to bullous pemphigoid,” J. Exp. Med., 1997, 186(5):777-783.
Lobsiger et al., “Altered axonal architecture by removal of the heavily phosphorylated neurofilament tail domains strongly slows superoxide dismutase 1 mutant-mediated ALS,” Proc. Natl. Acad. Sci. USA, 2005, 102(29):10351-10356.
Lopez et al, “Acidic pH increases the avidity of FcγR for immune complexes,” Immunology, 1999,98:450-455.
Ludemann et al., “O-glycosylation of the tail domain of neurofilament protein M in human neurons and in spinal cord tissue of a rat model of amyotrophic lateral sclerosis (ALS),” J. Biol. Chem., 2005, 280(36):31648-31658.
Manzi et al., “Inflammation-mediated rheumatic diseases and atherosclerosis,” Ann. Rheum. Dis., 2000, 59(5):321-325.
Marino et al., “Prevention of systemic lupus erythematosus in MRL/Ipr mice by administration of an immunoglobulin-binding peptide,” Nat. Biotechnol., 2000, 18:735-739.
Martin et al., “Crystal structure at 2.8 A of an FcRn/heterodimeric Fc complex: mechanism of pH-dependent binding,” Mol. Cell, 2001, 7:867-877.
Mathiot et al., “In vitro Inhibition of tumor B cell growth by IgG-BF-producing FcγRII+ T Cell hybridoma and by immunoglobulin G-binding factors,” Immunol. Res., 1992, 11:296-304.
McCall and Easterbrook-Smith, “The presence of histidine residues at or near the C1q binding site of rabbit immunoglobulin G,” Biochim. Biophys. Acta, 1987, 912:9-15.
Millecamps et al., “Synaptic sprouting increases the uptake capacities of motoneurons in amyotrophic lateral sclerosis mice, ”Proc. Natl. Acad. Sci. USA, 2001, 98(13):7582-7587.
Mizutani et al., “Development and characterization of monoclonal antiplatelet autoantibodies from autoimmune thrombocytopenic purpura-prone (NZW×BXSB)F1 mice,” Blood, 1993, 82:837-844.
Mohamed et al., “Immunoglobulin Fcγ receptor promotes immunoglobulin uptake, immunoglobulin-mediated calcium increase, and neurotransmitter release in motor neurons,” J. Neurosci. Res., 2002, 69:110-116.
Moller and Christiansen, “Fc-mediated immune precipitation. III. Visualization by electron microscopy,” Immunology, 1983, 48:469-476.
Moller and Pedersen, “Fc-mediated immune precipitation IV. Antigen dependency and specificity,” Immunology, 1983, 48:477-488.
Moller and Steensgaard, “Fc-mediated immune precipitation. II. Analysis of precipitating immune complexes by rate-zonal ultracentrifugation,” Immunology, 1979, 38:641-648.
Moller, “Fc-mediated immune precipitation. I. A new role of the Fc-portion of IgG,” Immunology, 1979, 38:631-640.
Morrison et al., “Early and selective pathology of light chain neurofilament in the spinal cord and sciatic nerve of G86R mutant superoxide dismutase transgenic mice,” Exp Neurology, 2000, 165:207-220.
Nardella et al., “IgG rheumatoid factors and staphylococcal protein A bind to a common molecular site on IgG,” J. Exp. Med., 1985, 162:1811-1824.
Nardella et al., “T15 group A streptococcal Fc receptor binds to the same location on IgG as staphylococcal protein A and IgG rheumatoid factors,” J. Immunol., 1987, 138:922-926.
Nguyen et al., “Exacerbation of motor neuron disease by chronic stimulation of innate immunity in a mouse model of amyotrophic lateral sclerosis,” J. Neurosci., 2004, 24(6):1340-1349.
Nielsen et al. “Release of leukotriene B4 and 5-hydroxyeicosatetraenoic acid during phagocytosis of artificial immune complexes by peripheral neutrophils in chronic inflammatory bowel disease,” Clin. Exp. Immunol., 1986, 65:465-471.
Obal et al., “Recruitment of activated microglia cells in the spinal cord of mice by ALS IgG,” Neuroreport, 2001, 12(11):2449-2452.
O'Brien et al, “The effects of histidine residue modifications on the immune precipitating ability of rabbit IgG,” Arch Biochem Biophys., 1994, 310:25-31.
Orr et al., “A possible role for humoral immunity in the pathogenesis of Parkinson's disease,” Brain, 2005, 128:2665-2674.
Padlan, “Anatomy of the antibody molecule,” Mol. Immunol., 1994, 31:169-217.
Pasceri and Yeh, “A tale of two diseases. Atherosclerosis and rheumatoid arthritis,” Circulation, 1999, 100:2124-2126.
Peress et al., “Identification of FcγRI, II and III on normal human brain ramified microglia and on microglia in senile plaques Alzheimer's disease,” J. Neuroimmunol., 1993, 48:71-80.
Petkova et al., “Human antibodies induce arthritis in mice deficient in the low-affinity inhibitory IgG receptor Fc gamma RIIB” J. Exp. Med., 2006, 203(2):275-280.
Poston, “A mechanism for demyelinating disease?” Lancet, 1984, 1:1268-1271.
Procaccia et al., “Circulating immune complexes in serum and in cerebrospinal fluid of patients with multiple sclerosis,” Acta Neurol. Scand., 1988, 77:373-381.
Procaccia et al., “Detection of rheumatoid factors of different isotypes and of circulating immune complexes in patients with inflammatory bowel disease,” Boll Ist. Sieroter. Milan, 1990, 69:413-421.
Pullen et al., “Passive transfer of purified IgG from patients with amyotrophic lateral sclerosis to mice results in degeneration of motor neurons accompanied by Ca2+ enhancement,” 2004 Acta Neuropathol., 107:35-46.
Raghavan and Bjorkman, “Fc receptors and their interactions with immunoglobulins,” Annu Rev Dev Biol., 1996, 12:181-220.
Rakhit et al., “Monomeric Cu,Zn-superoxide dismutase is a common misfolding intermediate in the oxidation models of sporadic and familial amyotrophic lateral sclerosis,” J. Biol. Chem., 2004, 279(15):15499-15504.
Ratcliffe et al., “Immunocytochemical detection of Fcγ receptors in human atherosclerotic lesions,” Immunol. Lett., 2001, 77:169-174.
Ravetch, “A full complement of receptors in immune complex diseases,” J Clin Invest., 2002, 110:1759-1761.
Ray and Lansbury, Jr., “A possible therapeutic target for Lou Gehrig's disease,” Proc. Natl. Acad. Sci. USA, 2004, 101(16):5701-5702.
Riederer et al., “Human immunoglobulins and Fc fragments promote microtubule assembly via tau proteins and induce conformational changes of neuronal microtubules in vitro,” NeuroReport, 2003, 14:117-121.
Ripps et al., “Transgenic mice expressing an altered murine superoxide dismutase gene provide an animal model of amyotrophic lateral sclerosis,” Proc. Natl. Acad. Sci. USA, 1995, 92:689-693.
Rodahl et al., “Participation of antigens related to the psoriasis associated antigen, pso p27, in immune complex formation in patients with ankylosing spondylitis,” Ann. Rheum. Dis., 1988, 47:628-633.
Rodriguez et al., “Destabilization of apoprotein in insufficient to explain Cu,Zn-superoxide dismutase-linked ALS pathogenesis,” Proc. Natl. Acad. Sci. USA, 2005, 102(30):10516-10521.
Roher et al., “P3-Amyloid-(1-42) is a major component of cerebrovascular amyloid deposits: implications for the pathology of Alzheimer disease,” Proc. Natl. Acad. Sci. USA, 1993, 90:10836-10840.
Ross and Poirier, “Protein aggregation and neurodegenerative disease,” Nature Med., 2004, S10-S17.
Roy et al., “Glutamate potentiates the toxicity of mutant Cu/Zn-superoxide dismutase in motor neurons by postsynaptic calcium-dependent mechanisms,” J. Neurosci., 1998, 18(23):9673-9684.
Ruangjirachuporn et al., “Circulating immune complexes in serum from patients with dengue haemorrhagic fever,” Clin. Exp. Immunol., 1979 36:46-53.
Rubinstein et al., “Anti-platelet antibody interactions with Fc gamma receptor,” Semin. Thromb. Hemost., 1995, 21:10-22.
Sahu et al., “Binding kinetics, structure-activity relationship, and biotransformation of the complement inhibitor compstatin,” J. Immunol., 2000, 165:2491-2499.
Saphire et al., “A new look at rheumatoid factor,” Cutting Edge Reports, from http://www.rheuma21st.com—pp. 1-9, 2001.
Saphire et al., “Crystal structure of a neutralizing human IgG against HIV-1: A template for vaccine design,” Science, 2001, 293:1155-1159.
Sasso et al., “Antigenic specificities of human monoclonal and polyclonal IgM rheumatoid factors,” J. Immunol., 1988, 140(9):3098-3107.
Schenk and Yednock, “Immunization with amyloid-β attenuates Alzheimer's-disease-like pathology in the PDAPP mouse,” Nature, 1999, 400:173-177.
Schenk and Yednock, “The role of microglia in Alzheimer's disease: friend or foe?” Neurobiol. Aging, 2002, 23:677-679.
Schlesinger and Chapman, “Influence of the human high-affinity IgG receptor FcgammaRI (CD64) on residual infectivity of neutralized dengue virus,” Virology. vol. 260, No. 1 (July 1999), pp. 84-88.
Shevtsova et al., “Promoters and serotypes: targeting of adeno-associated virus vectors for gene transfer in the rat central nervous system in vitro and in vivo,” Exp. Physiol., 2004, 90:53-59.
Shibata et al., “Presence of Cu/Zn SOD immunoreactivity in Neuronal Hyaline Inclusions in Spinal Cords from mice carrying a transgene for G93A Mutant Human SOD,” Acta Neuropathol., 1998, 95:136-142.
Shields et al., “High resolution mapping of the binding site on human IgG1 for FcγRI, FcγRII, FcγRIII, and FcRn and design of IgG1 variants with improved binding to the FcγR,” J. Biol. Chem., 2001, 276(9):6591-6604.
Sindic et al., “The binding of myelin basic protein to the Fc region of aggregated IgG and to immune complexes,” Clin. Exp. Immunol., 1980, 41:1-7.
Singh et al., “Reexamination of the mechanism of hydroxyl radical adducts formed from the reaction between familial amyotrophic lateral sclerosis-associated Cu,Zn superoxide dismutase mutants and H2O2,” Proc. Natl. Acad. Sci. USA, 1998, 95:6675-6680.
Smith et al., “Autoimmunity and ALS,” Neurology, 1996, (4 Suppl 2):S40-5; discussion S45-6. Review.
Smith et al., “Cytotoxicity of immunoglobulins from amyotrophic lateral sclerosis patients on a hybrid motoneuron cell line,” Proc. Natl. Acad. Sci. USA, 1994, 91:3393-3397.
Sohi et al., “Crystallization of a complex between the Fab fragment of a human immunoglobulin M (IgM) rheumatoid factor (RF-AN) and the Fc fragment of human IgG4,” Immunol., 1996, 88:636-641.
Solomon, “Intravenous immunoglobulin and Alzheimer's disease immunotherapy,” Curr Opin. Mol. Therapeutics, 9(1):79-85, 2007.
Sondermann et al., “Crystal structure of the soluble form of the human Fcγ-receptor IIb: a new member of the immunoglobulin superfamily at 1.7.ANG. resolution,” EMBO J., 1999, 18(5):1095-1103.
Sondermann et al., “The 3.2-.ANG. crystal structure of the human IgG1 Fc fragment-FcγRIII complex,” Nature, 2000, 406:267-273.
Song et al., “Fcγ receptor I- and III-Mediated macrophage inflammatory protein 1α induction in primary human and murine microglia,” Infect. Immun., 2002, 70(9):5177-5184.
Stasi and Provan, “Management of immune thrombocytopenic purpura in adults,” Mayo Clin Proc. 79(4):504-522, 2004.
Steif et al, “Subunit interactions provide a significant contribution to the stability of the dimeric four-α-helical-bundle protein ROP,” Biochem., 1993,32:3867-3876.
Stieber et al., “Disruption of the structure of the Golgi apparatus and the function of the secretory pathway by mutants G93A and G85R of Cu, Zn superoxide dismutase (SOD1) of familial amyotrophic lateral sclerosis,” J. Neurol. Sci., 2004, 219:45-53.
Stone et al., “The Fc binding site for streptococcal protein G is in the Cγ2-Cγ3 interface region of IgG and is related to the sites that bind staphylococcal protein A and human rheumatoid factors,” J. Immunol., 1989, 143(2):565-570.
Sulica et al. “Effect of protein A of Staphylococcus aureus on the binding of monomeric and polymeric IgG to Fc receptor-bearing cells,” Immunology, 1979, 38:173-179.
Takai, “Fc Receptors and their role in immune regulation and autoimmunity,” 2005, J Clin Immunology, 2005, 25: 1-18.
Tamura et al., “The F(ab′)2 fragment of an Aβ-specific monoclonal antibody reduces Aβ deposits in the brain,” Neurobiol. Disease, 2005, 20:541-549.
Termaat et al., “Anti-DNA antibodies can bind to the glomerulus via two distinct mechanisms,” Kidney Int., 1992, 42:1363-1371.
Theodore et al., “Targeted overexpression of human 1-synuclein triggers microglial activation and adaptive immune response in a mouse model of Parkinson disease,” J. Neuropath. Exp. Neurol., 67(12): 1149-1158, 2008.
Theofilopoulos et al., “The Raji Cell radioimmune assay for dectecting immune complexes in human sera,” J. Clin. Invest, 1976, 57:169-182.
Tiwari and Hayward, “Familial amyotrophic lateral sclerosis mutants of copper/zinc superoxide dismutase are susceptible to disulfide reduction,” J. Biol. Chem., 2003, 278(8):5984-5992.
Tiwari et al., “Aberrantly increased hydrophobicity shared by mutants of Cu,Zn-superoxide dismutase in familial amyotrophic lateral sclerosis,” J. Biol. Chem., 2005, 280(33):29771-29779.
Trojanowski et al., “Altered Tau and neurofilament proteins in neuro-degenerative diseases: diagnostic implications for Alzheimer's disease and Lewy body dementias,” Brain Pathology, 1993, 3:45-54.
Tummala et al., “Inhibition of chaperone activity is a shared property of several Cu, Zn-SOD mutants that cause ALS,” J Biol Chem., 2005, 290:17725-17731.
Urushitani et al., “Chromogranin-mediated section of mutant SOD Proteins linked to ALS,” Nature Neuroscience, 2006 9:108-118.
Valentine and Hart, “Misfolded CuZnSOD and amyotrophic lateral sclerosis,” Proc. Natl. Acad. Sci. USA, 2003, 100(7):3617-3622.
Valentine et al., “Copper-zinc superoxide dismutase and amyotrophic lateral sclerosis,” Annu. Rev. Biochem., 2005, 74:563-593.
Vasileva and Jessberger, “Precise hit: adeno-associated virus in gene targeting,” Nature Microbiology Rev., 2005, 3:837-847.
Vaughn and Bjorkman, “Structural basis of pH-dependent antibody binding by the neonatal Fc receptor,” Structure, 1998, 6:63-73.
Vedeler et al., “Fc receptors for immunoglobulin G—a role in the pathogenesis of Guillain-Barre syndrome and multiple sclerosis,” J. Neuroimmunol., 2001, 118:187-193.
Verdini and Viscomi, “Synthesis, resolution, and assignment of configuration of potent hypotensive retro-inverso bradykinin potentiation peptide 5a(BPPP₅ₐ) analogues,” J. Chem. Soc. Perkin Trans., 1985, I:697-701.
Verkhivker et al., “Monte Carlo simulations of the peptide recognition at the consensus binding site of the constant fragment of human immunoglobulin G: the energy landscape analysis of a hot spot at the intermolecular interface,” Proteins, 2002, 48(3):539-557.
Walker et al., “Using protein-based motifs to stabilize peptides,” J Peptide Res., 2003; 62:214-226.
Wallace et al., “Role of Fcγ receptors in cancer and infectious disease,” J. Leukocyte Biol., 1994, 55:816-826.
Wang et al., “Copper-binding-site-null SOD1 causes ALS in transgenic mice: aggregates of non-native SOD1 delineate a feature” Human Molecular Genetics, 2003, vol. 12, No. 21 2753-2764.
Webster et al., “Antibody-Mediated phagocytosis of the amyloid b-peptide in micoglia is differentially modulated by C1q,” J Immunol., 2001, 166:7496-7503.
West, Jr. and Bjorkman, “Crystal structure and immunoglobulin G binding properties of the human major histocompatibility complex-related Fc receptor,” Biochemistry, 2000, 39:9698-9708.
Wines et al., “The IgG Fc contains distinct Fc receptor (FcR) binding sites: the leukocyte receptors FcγRI and FcγRIIa bind to a region in the Fc distinct from that recognized by neonatal FcR and protein A,” J. Immunol., 2000, 164: 5313-5318.
Wines et al., “Soluble FcgammaRIIa inhibits rheumatoid factor binding to immune complexes,” Immunol., 2003, 109:246-254.
Witz and Ran, “FcR may function as a progression factor of nonlymphoid tumors,” Immunol. Res., 1992, 11:283-295.
Yim et al., “A familial amyotrophic lateral sclerosis-associated A4V Cu, Zn-superoxide dismutase mutant has a lower Km for hydrogen peroxide. Correlation between clinical severity and the Km value,” J. Biol. Chem., 1997, 272:8861-8863.
Yim et al., “A gain-of-function of an amyotrophic lateral sclerosis-associated Cu,Zn-superoxide dismutase mutant: An enhancement of free radical formation due to a decrease in Km for hydrogen peroxide,” Proc. Natl. Acad. Sci. USA, 1996, 93:5709-5714.
Yim et al., “Copper, zinc superoxide dismutase catalyzes hydroxyl radical production from hydrogen peroxide,” Proc. Natl. Acad. Sci. USA, 1990, 87:5006-5010.
Yim et al., “Enzyme Function of Copper, zinc superoxide dismutase as a free radical generator,” J. Biol. Chem., 1993, 268(6):4099-4105.
Zack et al., “Localization of an Fc-binding reactivity to the constant region of human IgG4,” J. Immunol., 1995, 155:5057-5063.
Zhang et al, “Bicarbonate Enhances Peroxidase Activity of Cu,Zn-Superoxide Dismutase,” J Biol Chem., 2002, 277:1013-1020.
Zhang et al., “Conformational changes of the flavivirus E glycoprotein,” Structure, 2004 12:1607-1618.
Zhang et al., “Bicarbonate Enhances the Hydroxylation, Nitration, and Peroxidation Reactions Catalyzed by Copper, Zinc Superoxide Dismutase,” J. Biol. Chem., 2000, 275:14038-14045.
Zhao et al., “Activated microglia initiate motor neuron injury by a nitric oxide and glutamate-mediated mechanism,” J. Neuropathol. Exp. Neurol., 2004, 63(9):964-977.
International Search Report and Written Opinion for PCT/US2009/050966, mailed Feb. 22, 2010, 7 pages.
International Preliminary Report on Patentability for PCT/US2009/050966, issued Jan. 18, 2011, 5 pages.
European Search Report for European Application No. 09798797.8, dated Jun. 22, 2012, 6 pages.
Search Report and Written Opinion by the Hungarian Intellectual Property Office for Singapore Application No. 201100335-7, mailed Feb. 1, 2013, 12 pages. Written Opinion for Singapore App. No. 201100335-7, mailed Nov. 28, 2013, 9 pages.

1. A method for inhibiting immune complex formation in a subject who has been diagnosed as having or is suspected of having an Ebola virus infection, said method comprising administering to said subject a composition comprising a purified polypeptide, said polypeptide comprising the amino acid sequence Xaa-Pro-Pro-Asp-Cys-Ala-Trp-His-Leu-Gly-Glu-Leu-Val-Trp-Cys-Thr (SEQ ID NO: 19), wherein Xaa is any amino acid, and wherein said immune complex formation is associated with Antibody Dependent Enhancement (ADE) of Ebola Virus Disease or infection by Ebola Virus (EBOV), or contributes to the enhancement of an EBOV infection.

2. The method of claim 1, further comprising monitoring said subject for one or more clinical, histiopathological or molecular characteristics of hemorrhagic fever.

3. The method of claim 2, wherein said one or more clinical, histiopathological, or molecular characteristics of hemorrhagic fever are selected from the group consisting of a decrease in platelets, hemoconcentration, or an increase in FcγR+ effector cells.

4. The method of claim 1, wherein the inhibited immune complex formation is Fc-mediated immune complex formation.

5. (canceled)

6. The method of claim 1, wherein the subject has been diagnosed with having or is suspected of having Ebola virus Zaire (Zaire ebolavirus; ZEBOV); Sudan virus (Sudan ebolavirus; SUDV or SEBOV); Tai Forest virus (Taï Forest ebolavirus; TAFV); Bundibugyo virus (Bundibugyo ebolavirus; BDBV); or Reston virus (Reston ebolavirus; RESTV or REBOV).

7. The method of claim 1, wherein the subject has been diagnosed with having or is suspected of having a coinfection with a pathogen selected from the group consisting of human immunodeficiency virus (HIV), human cytomegalovirus (HCMV), herpes simplex virus (HSV), hepatitis C virus, human papilloma virus (HPV), Mycobacterium tuberculosis, malaria/Plasmodium falciparum, and Schistosoma haematobium.

8. The method of claim 1, wherein the subject is experiencing hemorrhagic symptoms.

9. (canceled)

10. The method of claim 1, wherein said polypeptide comprises the amino acid sequence Ala-Pro-Pro-Asp-Cys-Ala-Trp-His-Leu-Gly-Glu-Leu-Val-Trp-Cys-Thr (SEQ ID NO:20).

11. The method of claim 1, further comprising administering to the subject a composition comprising a therapeutic antibody.

12.-14. (canceled)

15. The method of any claim 1, wherein the subject is a human.

16. The method of claim 15, wherein the human was afflicted or is afflicted with a malaria infection.

17.-21. (canceled)

22. A method for inhibiting immune complex formation in a subject who has been diagnosed as having or is suspected of having an influenza virus infection, said method comprising administering to said subject a composition comprising a purified polypeptide, said polypeptide comprising the amino acid sequence Xaa-Pro-Pro-Asp-Cys-Ala-Trp-His-Leu-Gly-Glu-Leu-Val-Trp-Cys-Thr (SEQ ID NO: 19), wherein Xaa is any amino acid, and wherein said immune complex formation contributes to the enhancement of an influenza virus infection.

23. The method of claim 22, further comprising monitoring said subject for one or more clinical, histiopathological or molecular characteristics of influenza virus infection.

24. The method of claim 22, wherein the inhibited immune complex formation is Fc-mediated immune complex formation.

25. (canceled)

26. The method of claim 22, wherein said polypeptide comprises the amino acid sequence Ala-Pro-Pro-Asp-Cys-Ala-Trp-His-Leu-Gly-Glu-Leu-Val-Trp-Cys-Thr (SEQ ID NO:20).

27-31. (canceled)

31. The method of claim 22, wherein the subject is a human.

32. A method for inhibiting immune complex formation in a subject who has been diagnosed as having or is suspected of having a hepatitis virus infection, said method comprising administering to said subject a composition comprising a purified polypeptide, said polypeptide comprising the amino acid sequence Xaa-Pro-Pro-Asp-Cys-Ala-Trp-His-Leu-Gly-Glu-Leu-Val-Trp-Cys-Thr (SEQ ID NO: 19), wherein Xaa is any amino acid, and wherein said immune complex formation contributes to the enhancement of a hepatitis virus infection.

33. The method of claim 32, further comprising monitoring said subject for one or more clinical, histiopathological or molecular characteristics of a hepatitis virus infection.

34. The method of claim 32, wherein the inhibited immune complex formation is Fc-mediated immune complex formation.

35.-40. (canceled)

41. The method of claim 32, wherein the subject is a human.


SEQUENCE LISTING (60 sequences)

SEQ ID NO: 1 - protein, 13 aa, Artificial Sequence
  Description of Artificial Sequence Synthetic peptide
  MOD_RES (1)..(1): Any amino acid
  MOD_RES (4)..(4): Any amino acid
  MOD_RES (6)..(8): Any amino acid
  MOD_RES (13)..(13): Any amino acid
XCAXHXXXLVWCX

SEQ ID NO: 2 - protein, 13 aa, Artificial Sequence
  Description of Artificial Sequence Synthetic peptide
DCAWHLGELVWCT

SEQ ID NO: 3 - protein, 13 aa, Artificial Sequence
  Description of Artificial Sequence Synthetic peptide
DCAFHLGELVWCT

SEQ ID NO: 4 - protein, 13 aa, Artificial Sequence
  Description of Artificial Sequence Synthetic peptide
DCARHLGELVWCT

SEQ ID NO: 5 - protein, 13 aa, Artificial Sequence
  Description of Artificial Sequence Synthetic peptide
RCARHLGELVWCT

SEQ ID NO: 6 - protein, 13 aa, Artificial Sequence
  Description of Artificial Sequence Synthetic peptide
RCAWHLGELVWCT

SEQ ID NO: 7 - protein, 13 aa, Artificial Sequence
  Description of Artificial Sequence Synthetic peptide
RCAFHLGELVWCT

SEQ ID NO: 8 - protein, 11 aa, Artificial Sequence
  Description of Artificial Sequence Synthetic peptide
  MOD_RES (3)..(3): Phe, Tyr, Trp or Arg
CAXHLGELVWC

SEQ ID NO: 9 - protein, 12 aa, Artificial Sequence
  Description of Artificial Sequence Synthetic peptide
CAFHLGELVWCT

SEQ ID NO: 10 - protein, 12 aa, Artificial Sequence
  Description of Artificial Sequence Synthetic peptide
CAWHLGELVWCT

SEQ ID NO: 11 - protein, 12 aa, Artificial Sequence
  Description of Artificial Sequence Synthetic peptide
CARHLGELVWCT

SEQ ID NO: 12 - protein, 15 aa, Artificial Sequence
  Description of Artificial Sequence Synthetic peptide
  MOD_RES (1)..(1): Any amino acid
  MOD_RES (6)..(6): Trp, Tyr, Phe or Arg
XPPCAXHLGELVWCT

SEQ ID NO: 13 - protein, 15 aa, Artificial Sequence
  Description of Artificial Sequence Synthetic peptide
  MOD_RES (1)..(1): Any amino acid
XPPCAWHLGELVWCT

SEQ ID NO: 14 - protein, 15 aa, Artificial Sequence
  Description of Artificial Sequence Synthetic peptide
APPCAWHLGELVWCT

SEQ ID NO: 15 - protein, 15 aa, Artificial Sequence
  Description of Artificial Sequence Synthetic peptide
  MOD_RES (1)..(1): Any amino acid
XPPCARHLGELVWCT

SEQ ID NO: 16 - protein, 15 aa, Artificial Sequence
  Description of Artificial Sequence Synthetic peptide
APPCARHLGELVWCT

SEQ ID NO: 17 - protein, 15 aa, Artificial Sequence
  Description of Artificial Sequence Synthetic peptide
  MOD_RES (1)..(1): Any amino acid
XPPCAFHLGELVWCT

SEQ ID NO: 18 - protein, 15 aa, Artificial Sequence
  Description of Artificial Sequence Synthetic peptide
APPCAFHLGELVWCT

SEQ ID NO: 19 - protein, 16 aa, Artificial Sequence
  Description of Artificial Sequence Synthetic peptide
  MOD_RES (1)..(1): Any amino acid
XPPDCAWHLGELVWCT

SEQ ID NO: 20 - protein, 16 aa, Artificial Sequence
  Description of Artificial Sequence Synthetic peptide
APPDCAWHLGELVWCT

SEQ ID NO: 21 - protein, 16 aa, Artificial Sequence
  Description of Artificial Sequence Synthetic peptide
  MOD_RES (1)..(1): Any amino acid
XPPDCARHLGELVWCT

SEQ ID NO: 22 - protein, 16 aa, Artificial Sequence
  Description of Artificial Sequence Synthetic peptide
APPDCARHLGELVWCT

SEQ ID NO: 23 - protein, 16 aa, Artificial Sequence
  Description of Artificial Sequence Synthetic peptide
  MOD_RES (1)..(1): Any amino acid
XPPDCAFHLGELVWCT

SEQ ID NO: 24 - protein, 16 aa, Artificial Sequence
  Description of Artificial Sequence Synthetic peptide
APPDCAFHLGELVWCT

SEQ ID NO: 25 - protein, 16 aa, Artificial Sequence
  Description of Artificial Sequence Synthetic peptide
  MOD_RES (1)..(1): Any amino acid
XPPRCAWHLGELVWCT

SEQ ID NO: 26 - protein, 16 aa, Artificial Sequence
  Description of Artificial Sequence Synthetic peptide
APPRCAWHLGELVWCT

SEQ ID NO: 27 - protein, 16 aa, Artificial Sequence
  Description of Artificial Sequence Synthetic peptide
  MOD_RES (1)..(1): Any amino acid
XPPRCARHLGELVWCT

SEQ ID NO: 28 - protein, 16 aa, Artificial Sequence
  Description of Artificial Sequence Synthetic peptide
APPRCARHLGELVWCT

SEQ ID NO: 29 - protein, 16 aa, Artificial Sequence
  Description of Artificial Sequence Synthetic peptide
  MOD_RES (1)..(1): Any amino acid
XPPRCAFHLGELVWCT

SEQ ID NO: 30 - protein, 16 aa, Artificial Sequence
  Description of Artificial Sequence Synthetic peptide
APPRCAFHLGELVWCT

SEQ ID NO: 31 - protein, 15 aa, Artificial Sequence
  Description of Artificial Sequence Synthetic peptide
  MOD_RES (15)..(15): Any amino acid
CAWHLGELVWCTPPX

SEQ ID NO: 32 - protein, 15 aa, Artificial Sequence
  Description of Artificial Sequence Synthetic peptide
CAWHLGELVWCTPPA

SEQ ID NO: 33 - protein, 15 aa, Artificial Sequence
  Description of Artificial Sequence Synthetic peptide
  MOD_RES (15)..(15): Any amino acid
CARHLGELVWCTPPX

SEQ ID NO: 34 - protein, 15 aa, Artificial Sequence
  Description of Artificial Sequence Synthetic peptide
CARHLGELVWCTPPA

SEQ ID NO: 35 - protein, 15 aa, Artificial Sequence
  Description of Artificial Sequence Synthetic peptide
  MOD_RES (15)..(15): Any amino acid
CAFHLGELVWCTPPX

SEQ ID NO: 36 - protein, 15 aa, Artificial Sequence
  Description of Artificial Sequence Synthetic peptide
CAFHLGELVWCTPPA

SEQ ID NO: 37 - protein, 16 aa, Artificial Sequence
  Description of Artificial Sequence Synthetic peptide
  MOD_RES (16)..(16): Any amino acid
DCAWHLGELVWCTPPX

SEQ ID NO: 38 - protein, 16 aa, Artificial Sequence
  Description of Artificial Sequence Synthetic peptide
DCAWHLGELVWCTPPA

SEQ ID NO: 39 - protein, 16 aa, Artificial Sequence
  Description of Artificial Sequence Synthetic peptide
  MOD_RES (16)..(16): Any amino acid
DCARHLGELVWCTPPX

SEQ ID NO: 40 - protein, 16 aa, Artificial Sequence
  Description of Artificial Sequence Synthetic peptide
DCARHLGELVWCTPPA

SEQ ID NO: 41 - protein, 16 aa, Artificial Sequence
  Description of Artificial Sequence Synthetic peptide
  MOD_RES (16)..(16): Any amino acid
DCAFHLGELVWCTPPX

SEQ ID NO: 42 - protein, 16 aa, Artificial Sequence
  Description of Artificial Sequence Synthetic peptide
DCAFHLGELVWCTPPA

SEQ ID NO: 43 - protein, 16 aa, Artificial Sequence
  Description of Artificial Sequence Synthetic peptide
  MOD_RES (16)..(16): Any amino acid
RCAWHLGELVWCTPPX

SEQ ID NO: 44 - protein, 16 aa, Artificial Sequence
  Description of Artificial Sequence Synthetic peptide
RCAWHLGELVWCTPPA

SEQ ID NO: 45 - protein, 16 aa, Artificial Sequence
  Description of Artificial Sequence Synthetic peptide
  MOD_RES (16)..(16): Any amino acid
RCARHLGELVWCTPPX

SEQ ID NO: 46 - protein, 16 aa, Artificial Sequence
  Description of Artificial Sequence Synthetic peptide
RCARHLGELVWCTPPA

SEQ ID NO: 47 - protein, 16 aa, Artificial Sequence
  Description of Artificial Sequence Synthetic peptide
  MOD_RES (16)..(16): Any amino acid
RCAFHLGELVWCTPPX

SEQ ID NO: 48 - protein, 16 aa, Artificial Sequence
  Description of Artificial Sequence Synthetic peptide
RCAFHLGELVWCTPPA

SEQ ID NO: 49 - protein, 20 aa, Artificial Sequence
  Description of Artificial Sequence Synthetic peptide
  MOD_RES (4)..(4): Any amino acid
  MOD_RES (7)..(7): Any amino acid
  MOD_RES (9)..(11): Any amino acid
  MOD_RES (16)..(16): Any amino acid
WEAXCAXHXXXLVWCXKVEE

SEQ ID NO: 50 - protein, 20 aa, Artificial Sequence
  Description of Artificial Sequence Synthetic peptide
  MOD_RES (7)..(7): Arg, Trp, 5-Hydroxytryptophan (5-HTP), Tyr or Phe
WEADCAXHLGELVWCTKVEE

SEQ ID NO: 51 - protein, 20 aa, Artificial Sequence
  Description of Artificial Sequence Synthetic peptide
WEADCAWHLGELVWCTKVEE

SEQ ID NO: 52 - protein, 32 aa, Artificial Sequence
  Description of Artificial Sequence Synthetic polypeptide
  MISC_FEATURE (1)..(10): This region may encompass 0-10 residues wherein some positions may be absent
  MOD_RES (1)..(11): Any amino acid
  MOD_RES (14)..(14): Any amino acid
  MOD_RES (16)..(18): Any amino acid
  MISC_FEATURE (23)..(32): This region may encompass 0-10 residues wherein some positions may be absent
  MOD_RES (23)..(32): Any amino acid
XXXXXXXXXXXCAXHXXXLVWCXXXXXXXXXX

SEQ ID NO: 53 - protein, 22 aa, Artificial Sequence
  Description of Artificial Sequence Synthetic peptide
  MISC_FEATURE (1)..(5): This region may encompass 0-5 residues wherein some positions may be absent
  MOD_RES (1)..(5): Any amino acid
  MOD_RES (8)..(8): Phe or Arg
  MISC_FEATURE (18)..(22): This region may encompass 0-5 residues wherein some positions may be absent
  MOD_RES (18)..(22): Any amino acid
XXXXXCAXHLGELVWCTXXXXX

SEQ ID NO: 54 - protein, 22 aa, Artificial Sequence
  Description of Artificial Sequence Synthetic peptide
AAAAADCARHLGELVWCAAAAA

SEQ ID NO: 55 - protein, 17 aa, Artificial Sequence
  Description of Artificial Sequence Synthetic peptide
AARCARHLGELVWCTAA

SEQ ID NO: 56 - protein, 19 aa, Artificial Sequence
  Description of Artificial Sequence Synthetic peptide
AAADCAFWHLGELVWCTAA

SEQ ID NO: 57 - protein, 13 aa, Artificial Sequence
  Description of Artificial Sequence Synthetic peptide
  MOD_RES (1)..(1): Any amino acid or not present
  MOD_RES (4)..(4): Phe, Tyr, Trp, 5-Hydroxytryptophan (5-HTP) or Arg
  MOD_RES (6)..(6): Any amino acid
  MOD_RES (7)..(7): Gly or Ala
  MOD_RES (8)..(8): Glu or Ala
  MOD_RES (13)..(13): Any amino acid or not present
XCAXHXXXLVWCX

SEQ ID NO: 58 - protein, 20 aa, Artificial Sequence
  Description of Artificial Sequence Synthetic peptide
  MOD_RES (4)..(4): Any amino acid or not present
  MOD_RES (7)..(7): Phe, Tyr, 5-Hydroxytryptophan (5-HTP), Trp or Arg
  MOD_RES (9)..(9): Any amino acid
  MOD_RES (10)..(10): Gly or Ala
  MOD_RES (11)..(11): Glu or Ala
  MOD_RES (16)..(16): Any amino acid or not present
WEAXCAXHXXXLVWCXKVEE

SEQ ID NO: 59 - protein, 22 aa, Artificial Sequence
  Description of Artificial Sequence Synthetic peptide
  MISC_FEATURE (1)..(5): This region may encompass 0-5 residues wherein some positions may be absent
  MOD_RES (1)..(5): Any amino acid
  MOD_RES (6)..(6): Any amino acid or not present
  MOD_RES (9)..(9): Phe, Tyr, 5-Hydroxytryptophan (5-HTP), Trp or Arg
  MOD_RES (11)..(11): Any amino acid
  MOD_RES (12)..(12): Gly or Ala
  MOD_RES (13)..(13): Glu or Ala
  MISC_FEATURE (18)..(22): This region may encompass 0-5 residues wherein some positions may be absent
  MOD_RES (18)..(22): Any amino acid
XXXXXXCAXHXXXLVWCXXXXX

SEQ ID NO: 60 - protein, 22 aa, Artificial Sequence
  Description of Artificial Sequence Synthetic peptide
  MISC_FEATURE (1)..(5): This region may encompass 0-5 residues wherein some positions may be absent
  MOD_RES (1)..(5): Any amino acid
  MOD_RES (8)..(8): Trp, Tyr, Phe, 5-Hydroxytrphophan (5-HTP), 5-hydroxytryptamine (5-HT) or another amino acid derivative
  MISC_FEATURE (18)..(22): This region may encompass 0-5 residues wherein some positions may be absent
  MOD_RES (18)..(22): Any amino acid
XXXXXCAXHLGELVWCTXXXXX